(19) United States
(12) Patent Application Publication
(10) Pub. No.: US 2010/0279289 A1
(43) Pub. Date: Nov. 4, 2010

(54) SIZE-DEPENDENT BIOLOGICAL EFFECT OF NANOPARTICLES

(76) Inventor: Fanqing Frank Chen, Moraga, CA (US)

Correspondence Address: Weaver Austin Villeneuve & Sampson LLP, P.O. BOX 70250, OAKLAND, CA, 94612-0250, US

(21) Appl. No.: 12/601,747

(22) PCT Filed: May 23, 2008
(86) PCT No.: PCT/US08/06701

§ 371 Date: Jun. 1, 2010

(51) Int. Cl. C12Q 1/68

(52) U.S. Cl. 435/6

(57) ABSTRACT

Nanoparticles are used increasingly in consumer products and biomedical applications. Yet the cellular interaction mechanism at the molecular level is not well understood for nanomaterials of different size, shape and surface chemistry. Gold nanoparticles (Au-NPs), which have been explored extensively for various applications in recent years, are used as the model system to help understand the size-dependent biological effects of nanoparticles. Jurkat cells treated with Au-NPs ranging from 2 nm to 200 nm were studied. Whole genome expression measurements indicate size-dependent effects, including linear scaling and threshold effects. In addition, a non-linear pattern of gene responses that persisted over time were observed in 20-40 nm Au-NP treated cells. Gene function, promoter, and pathway analyses reveal differential signaling processes that are correlated with nanoparticle sizes. The size may play a role in cellular sorting of naturally occurring particulates, particle interaction with the receptors, intracellular transportation, signaling and stress responses.

### CROSS-REFERENCE TO RELATED APPLICATIONS

This application claims benefit of and priority to U.S. Ser. No. 60/940,071, filed May 24, 2007, which is incorporated herein by reference in its entirety, including all supplemental data, for all purposes.

### STATEMENT OF GOVERNMENT SUPPORT

This invention was made during work supported by NASA JRI grant, CSF Prostate Cancer SPORE award (NIH Grant P50 CA89520), NIH grant R21CA95393-01, DOD grant BC045345, and DARPA grant F1ATA05252M001, and the U.S. Department of Energy under Contract No. DE-AC02-05CH11231. The government of the United States of America has certain rights in this invention.

### FIELD OF THE INVENTION

The present invention relates to nanoparticles and methods for determining their toxicity and potential biological effect on cells and organisms.

### BACKGROUND OF THE INVENTION

Nanomaterials are used in applications ranging from cosmetics and electronics to drug delivery vehicles (see, e.g., Powell and Kanarek (2006) Wmj 105: 16-20; Lin and Datar (2006) Natl Med J India 19: 27-32; Hardman (2006) Environ Health Perspect 114: 165-172). Yet, when their feature sizes fall in the 1-100 nm range that characterizes them as nanomaterials (see, e.g., Colvin (2003) Nat. Biotech. 21: 1166-1170; Haruta (2003) Chem Rec 3: 75-87; Oberdorster et al. (2005) Environ Health Perspect 113: 823-839; Borm (2002) Inhal Toxicol 14: 311-324), they have altered biological activities that are not manifest in the bulk forms. Nanomaterials have higher reactivity and a greater surface-to-mass ratio than more familiar the micro-sized particulate materials. Furthermore, the transport and persistence of nanomaterials in the cellular environment is drastically different from micro-sized particulate materials. For instance, the biomolecule-level size scale of nanomaterials allows for easier cell penetration. It has only been in recently that the biological mechanisms for interaction, uptake and metabolism of nanoparticles have begun to emerge (see, e.g., Derfus et al. (2004) Nano Letters 4: 11-18; Chithrani and Ghazani (2006) Nano Lett 6: 662-668 (2006); Borm et al. (2006) Toxicol Sci 90: 23-32). The data strongly suggest that physical properties, such as size, shape and surface charge, are significant factors for nanoparticle-specific cellular effects (Chithrani and Ghazani (2006) Nano Lett 6: 662-668 (2006); Sayes et al. (2004) Nano Letters 4, 1881-1887; Goodman et al. (2004) Bioconjug Chem 15: 897-900). Although limited scale gene expression analyses have been performed (see, e.g., Matsusaki et al. (2005) Nano Lett. 5(11): 2168 -2173; Zhang et al. (2006) Nano Lett 6: 800-808; Ding et al. (2005) Nano Lett 5: 2448-2464), the exact gene and protein level mechanisms remain poorly defined. It is critical to understand the biological effects of nanomaterials at the molecular level, in relation to their physico-chemical properties, so that they can be better manipulated and optimized for biomedical applications, as well as for responsible/rational assessment of health, occupational and environmental risks.

In recent years, significant developments in gold nanoparticle (Au-NP) synthesis have shifted research efforts toward biomedical and clinical applications. Their inherent size, shape and optical properties make them particularly well suited for in vivo use in detection and treatment platforms (see, e.g., Pedroso and Guillen (2006) Comb Chem High Throughput Screen 9: 389-397; Loo et al. (2005) Nano Lett 5: 709-711; Taton et al. (2000) Science 289: 1757-1760; Han et al. (2006) J Am Chem Soc 128: 4954-4955; Elghanian et al. (1007) Science 277: 1078-1081; Cao et al. (2002) Science 297: 1536-1540; Sonnichsen et al. (2005) Nat Biotechnol 23: 741-745; Liu et al. (2006) Nature Nanotechnology 1: 47-52; Liu et al. (2007) J. Nanosci. Nanotechnol. 7: 1-8).

### SUMMARY OF THE INVENTION

Gold nanoparticle of nine different sizes in the same size range as molecular and cellular structures in the cell were administered to cell lines and resulting size-dependent changes in gene expression were determined. Four different patterns of size-dependent gene expression were identified and can be used to screen nanoparticles to identify which biological effects of the particles are caused by particle size per se, and which biological

Accordingly, in certain embodiments, methods are provided for identifying size-dependent biological effects of a nanoparticle on a cell. The methods typically involve contacting the cell with the nanoparticle; measuring levels of gene expression in the cell of at least two genes, preferably at least 3 genes, more preferably at least 4 genes, still more preferably at least 5, 8, 10, 15, or 20 genes, in certain embodiments, at least 5%, at least 10%, at least 25%, at least 50%, at least 75%, at least 80%, or all of the genes, in certain found in one or more of Pattern Set 1, Pattern Set 2, Pattern Set 3, or Pattern Set 4; where changes in expression level(s) of the genes consistent with Pattern 1, Pattern 2, Pattern 3, or Pattern 4 is an indicator of size effects of the nanoparticle on the cell; and where changes in expression level deviating from Pattern 1, Pattern 2, Pattern 3, and Pattern 4 is an indicator of biological effects that are not solely due to nanoparticle size. In certain embodiments the method involves measuring expression levels for all of the genes found in Pattern Set 1, and/or Pattern Set 2, and/or Pattern Set 3, and/or Pattern Set 4. In certain embodiments changes in expression level of the genes consistent with Pattern 1, Pattern 2, Pattern 3, or Pattern 4 indicates that the expression levels of at least 5%, at least 10%, at least 25%, at least 50%, at least 75%, at least 80%, or all of the measured genes is upregulated or downregulated as shown in Table 2 for pattern 1, Table 3 for pattern 2, Table 4 for pattern 3, or Table 5 for pattern 4, for particles of the same average size. In certain embodiments changes in expression level of the genes consistent with Pattern 1, Pattern 2, Pattern 3, or Pattern 4 indicates that the magnitude of upregulation or downregulation of the measured pattern set genes is comparable to the average magnitude shown in Pattern 1, Pattern 2, Pattern 3, or Pattern 4 for particles of the same size. In certain embodiments changes in expression level of the genes consistent with Pattern 1, and/or Pattern 2, and/or Pattern 3, and/or Pattern 4 indicates that there is no statistically significant difference (e.g., at the 90%, 95%, 98% or 99% confidence level) in the expression level of the measured genes from the average expression levels comprising Pattern 1, pattern 2, pattern 3, or pattern 4 for particles of the same average size. In certain embodiments the nanoparticle is a nanoparticle selected from the group consisting of a metal nanoparticle, a semiconductor nanoparticle, a polymeric nanoparticle, a dendromeric nanoparticle, a ceramic nanoparticle, a mineral nanoparticle, and a lipidic nanoparticle. In certain embodiments the nanoparticle is a nanoparticle formulated for drug delivery (e.g., a polymeric nanoparticle (PNP), a liposome, etc.). In certain embodiments the nanoparticle further comprises a pharmaceutical or other reagent. In certain embodiments the contacting comprises contacting a cell in situ in a tissue or tissue section, or contacting a cell in culture. In certain embodiments the contacting comprises contacting comprises contacting a human cell. In certain embodiments the contacting comprises administering the nanoparticle to a non-human mammal, bacteria, protozoan, or the like. In certain embodiments the measuring comprises measuring gene expression using an array hybridization and/or a polymerase chain reaction (PCR) (e.g., RT-PCR).

In another embodiments, methods are provided for identifying biological effects of a nanoparticle on a cell where the effects are not solely due to the size of the nanoparticle. The methods typically involve contacting the cell with the nanoparticle; measuring levels of gene expression in the cell where changes in expression level of genes other than genes found in one or more of Pattern Set 1, Pattern Set 2, Pattern Set 3, or Pattern Set 4, or changes of expression level of genes in one or more of Pattern Set 1, Pattern Set 2, Pattern Set 3, or Pattern Set 4 deviating from Pattern 1, Pattern 2, Pattern 3, and Pattern 4 is an indicator of biological effects that are not solely due to nanoparticle size. In certain embodiments the measuring comprises measuring at least two genes, preferably at least 3 genes, more preferably at least 4 genes, still more preferably at least 5, 8, 10, 15, or 20 genes, in certain embodiments, at least 5%, at least 10%, at least 25%, at least 50%, at least 75%, at least 80%, or all of the genes, found in Pattern Set 1, and/or Pattern Set 2, and/or Pattern Set 3, and/or Pattern Set 4. In certain embodiments the measuring comprises measuring all of the genes found in Pattern Set 1, Pattern Set 2, Pattern Set 3, and Pattern Set 4. In certain embodiments the measuring comprises measuring expression levels of at least two, preferably at least 3, 4, or 5, more preferably at least 10, 15, 20, 50, 100, or 200 genes not found in pattern set 1, pattern set 2, pattern set 3, or pattern set 4. In certain embodiments changes in expression level of the genes consistent with Pattern 1, Pattern 2, Pattern 3, or Pattern 4 indicates that the expression level at least at least 5%, at least 10%, at least 25%, at least 50%, at least 75%, at least 80%, or all of the measured genes is upregulated or downregulated as shown in Table 2 for pattern 1, Table 3 for pattern 2, Table 4 for pattern 3, or Table 5 for pattern 4, for particles of the same average size . In certain embodiments changes in expression level of the genes consistent with Pattern 1, Pattern 2, Pattern 3, or Pattern 4 indicates that the magnitude of upregulation or downregulation of the measured pattern set genes is comparable to the average magnitude shown in Pattern 1, Pattern 2, Pattern 3, or Pattern 4 for particles of the same size. In certain embodiments changes in expression level of the genes consistent with Pattern 1, and/or Pattern 2, and/or Pattern 3, and/or Pattern 4 indicates that there is no statistically significant difference (e.g., at the 90%, 95%, 98% or 99% confidence level) in the expression level of the measured genes from the average expression levels comprising Pattern 1, pattern 2, pattern 3, or pattern 4 for particles of the same average size. In certain embodiments the nanopartidle is a nanoparticle selected from the group consisting of a metal nanoparticle, a semiconductor nanoparticle, a polymeric nanoparticle, a dendromeric nanoparticle, a ceramic nanoparticle, a mineral nanoparticle, and a lipidic nanoparticle. In certain embodiments the nanoparticle is a nanoparticle formulated for drug delivery (e.g., a polymeric nanoparticle (PNP), a liposome, etc.). In certain embodiments the nanoparticle further comprises a pharmaceutical or other reagent. In certain embodiments the contacting comprises contacting a cell in situ in a tissue or tissue section, or contacting a cell in culture. In certain embodiments the contacting comprises contacting comprises contacting a human cell. In certain embodiments the contacting comprises administering the nanoparticle to a non-human mammal, bacteria, protozoan, or the like. In certain embodiments the measuring comprises measuring gene expression using an array hybridization and/or a polymerase chain reaction (PCR) (e.g., RT-PCR).

In certain embodiments methods are also provided for identifying genes whose expression is altered by nanoparticle size. The methods typically involve contacting a cell with a nanoparticles having different sizes; and identifying genes whose expression level differs when exposed to at least two different size nanoparticles. In certain embodiments nanoparticles range in average size from about 1 nm to about 500 nm, preferably from about 2 nm to about 200 nm. In certain embodiments the cell is a mammalian cell. In certain embodiments the cell is not a mammalian cell. In certain embodiments the cell is an invertebrate cell, a bacterial cell, or a protozoan cell. In certain embodiments the contacting comprises administering said nanoparticles to a non-human mammal or other non-human animal. In certain embodiments the contacting comprises administering the nanoparticles to a cell in culture. In certain embodiments the method further comprises recording the identified genes on paper and/or on a computer readable medium (e.g., magnetic media, optical media, etc.).

Methods are also provided for assessing the cytotoxic effect of a nanomaterial upon a cell. The methods typically involve exposing the cell to a nanomaterial; detecting from the cell, the pattern of gene amplification or gene expression for at least one gene set forth in Tables 1, 2, 3, 4, 5, and/or at least one gene set forth in FIGS. 4E, 4F, 4G, or 4H, and/or in pattern set 1, pattern set 2, pattern set 3, pattern set 4, or pattern set 5 in response to the exposure; identifying at least a two-fold change in gene expression of the gene(s); whereby, when the two-fold, or greater, change in gene expression is identified, this is an indicator that the nanoparticle is cytotoxic to the cell. In certain embodiments the detecting comprises use a of methodology selected from the group consisting of transcription profiling, the measurement of phenotypic changes in large populations of cells by high content analysis, gene expression array analysis in exposed cells, measuring mRNA level changes, promoter analysis, chemically induced toxicity, 2D gel electrophoresis, mass spectrometry, and reverse phase protein lysate arrays for protein.

In various embodiments methods are provided for measuring size dependent biological effect(s) of nanoparticles on a cell. The methods typically involve exposing a cell to a nanoparticle, performing gene expression profiles and gene function, promoter and pathway analyses on the cell after exposure to the nanoparticle(s) and identifying and comparing the patterns that emerge as compared to size-dependent patterns I, II, III and IV shown in FIGS. 4A, 4B, 4C, and/or 4D, where a change in expression profile consistent with the patterns is an indicator of size dependent biological effect of the nanoparticle on the cell. In certain embodiments a greater than 5%, 10%, 15%, 20%, 25%, or 50% change in the up or down regulation of one or more particular gene(s) is an indicator that more specific toxicology studies of the nanoparticle are desirable. In certain embodiments the cell exposure is carried out in 3D tissue culture environments. In certain embodiments the cell is mammalian or bacterial.

### DEFINITIONS

The term “nanoparticle” refers to any nano-sized particle, regardless of shape, including but not limited to, metal particles (e.g., gold), any metal oxide, semiconductor or radionuclide particle, semiconductor nanocrystals, dendrimers, liposomes, and carbon-based nanomaterials, such as carbon nano-tubes, nano-onions, fullerenes, and the like. Typically nanoparticles have a characteristic size (e.g., diameter) of less than about 500 nm, preferably less than about 400 nm or 300 nm. In various embodiments nanoparticle range in size from about 0.5 nm, 1 nm, 2 nm, 5 nm, or 10 nm to about 1 nm to about 200 nm, 150 nm, 100 nm, 80 nm, 50, nm.

The term “pattern set” indicates a set or collection of genes that show altered expression when contacted with certain size nanoparticles and thereby generate a pattern of altered expression in response to those nanoparticles. Illustrative patterns sets 1-4 are shown herein in Table 1.

The term “indicator of biological effects” does not require that the result be dispositive. Thus, a result (e.g., gene expression pattern) that is an indicator of size-dependent nanoparticle effects does not require that the result must be a produced by size-dependent nanoparticle effects, but rather that the result is likely to be produced or at least influenced by size-dependent nanoparticle effects.

### BRIEF DESCRIPTION OF THE DRAWINGS

FIGS. 1A, 1B, 1C, and 1D illustrate the gold nanoparticles used in this study. FIG. 1A: The sizes of the Au-NPs are compared to the sizes of biological features within a cell. The blue arrow is the nuclear pore complex exclusion size (˜40 nm). The sizes of the Au-NPs are verified by TEM microscopy (FIGS. 1A and 1C). FIG. 1B: Example of TEM images of Au-NPs. Scale bar=20 nm for 2-30 nm nanoparticles, and scale bar=100 nm for 40-200 nm nanoparticles. FIGS. 1C and 1D: Histograms of the Au-NPs used in the study. Size distribution of the Au-NPs shows separation between different nanoparticle sizes.

FIGS. 2A-2F illustrate effects of nanoparticle exposure. FIG. 2A: Cell counts for Jurkat cells after treatment with 2 nm Au-NPs in various doses for 48 hours. The survival rate of the cells is mostly unaffected at doses used in the study. FIG. 2B: Treating Jurkat with Au-NPs cause slight increases in programmed cell death (apoptosis). Both 20-40 nm and 200 nm nanoparticles show increased programmed cell death. FIGS. 2C-2F: Number of genes that have expression changes in response to different sizes of Au-NP treatment. Genes in treated samples that have changed more than 1.5 fold from untreated control were counted (FIG. 2C: 2 hr 0.12 mg/L; FIG. 2D: 2 hr 1.2 mg/L; FIG. 2E: 8 hr 0.12 mg/L; FIG. 2F: 8 hr 1.2 mg/L).

FIG. 3, panels a, b, and c illustrate Principal Component Analysis (PCA) of gene expression profiles. Axis X: Component 1; Y: Component 2; Z: Component 3. Panel a: The overall PCA (center) result of the combination of two time-points (2 and 8 hours), two dosages [0.12 mg/L (10%) or 1.2 mg/L (100%)], and nine sizes (2, 5, 10, 15, 20, 30, 40, 80 and 200 nm). 70% variation of the dataset is captured in the first three dimensions shown in the center graph (Panel a). The data indicate that at 8 hrs, the differences between different sizes and doses are less prominent. There are size- and dose-dependent separations at 2 hr (Panels b and c). At 2 hours, there is clear separation both between the two dosage groups, and amongst the size variations within each dosage group. Panel b: PCA at 2 hr 0.12 mg/L (left). Panel c: PCA at 2 hr 1.2 mg/L (right). In panels b and c, size variation shows a linear relationship. The 2-40 nm size variant datasets project as a gradient on PCA for both the 0.12 mg/L (Panel b) and 1.2 mg/L (Panel c) dosages. 80 nm and 200 nm Au-NP treatments exhibited more PCA separation from the smaller 2-40 nm treatments in both dosage groups (Panels b and c).

FIGS. 4A-4H illustrate size-dependent gene expression patterns. FIGS. 4A-4D: Y-axis represents the fold changes of treated cell gene expression levels vs. the control cells, the changes are expressed as the ratio of treated/control in log2 (positive or negative numbers represent gene expression increase or decrease, respectively). FIG. 4A: Pattern I, a pseudo-linear gradient effect of gene expression change effects (from down-regulation at 2 nm treatment to near control levels at 40-80 nm treatment) is observed in 12.5% of the genes with varied expression. FIG. 4B: Pattern II, threshold effect elicited by Au-NP below 5 nm is the primary effect at 2 hour 0.12mg/L (15.1%). FIG. 4C: Pattern III, 10% gene expression changes peak at 20-40 nm and persist through 8 hrs for the 0.12 mg/L treatment (FIG. 4C, bottom panel). This effect is likely associated with uptaken and internalized Au nanoparticles, which was reported before (Chithrani et al. (2006) Nano Lett 6: 662-668). FIG. 4D: Pattern IV, another threshold effect occurs at 80-200 nm (>40 nm, the exclusion limit for nuclear pore complex (Rottmann and Luscher (2006) Curr Top Microbiol Immunol 302: 63-122), which persists with the 2-hr high-dose treatment (FIG. 4D, bottom panel). This pattern is also the most dominate pattern for the 2-hr high-dose treatment (data not shown) FIGS. 4E-4H: Heatmap of gene expression patterns correspond to 4A-4D, respectively. In pattern I (FIG. 4E), gene expression change with a liner gradient effect for 2-40 nm Au-NPs at the 2-hr low dose. In pattern II (FIG. 4F), 2 nm Au-NP treatment causes unique expression changes at 2-hr low dose. In pattern III (FIG. 4C top and bottom), gene expression changes for 20-40 nm peak pattern are time-persistent at low dose [FIG. 4G: left, and FIG. 4C top: 2-hr 0.12 mg/L; FIG. 4G right and FIG. 4D bottom: 8-hr 0.12 mg/L]. In pattern IV (FIG. 4D top and bottom), dose persistent expression change for genes affected by 80-200 nm Au-NPs [FIG. 4H left and FIG. 4D top: 2-hr 0.12 mg/L; FIG. 4H right and FIG. 4D bottom: 2-hr 1.2 mg/L (4b-vi)]. Functional analysis of the genes with the four size-dependent expression patterns is shown in Table 1. It is evident that different patterns exhibit enrichment in different function groups.

FIG. 5 illustrates a pathway analysis summary from Ingenuity Pathway Analysis. Illustrated at the top is a picture of Au-NPs of various sizes. The likely underlying signaling networks are divided into three separate size-dependent groups (a. 2 nm, b. 20-40 nm, c. 80-200 nm). The 5-15 nm group associated with Pattern I is not presented here. The cells respond to different sizes of Au-NPs using different size-dependent sorting strategies, and trigger different signaling pathways. The access of Au-NPs to the different cellular compartment could contribute to the differences as well. This size differentiation is probably part of the built-in circuitry for the cell surface receptors and intracellular sorting mechanisms that are preserved during evolution when dealing with environmental particulates. Pathways involving IL18, ASK1(MAP3K5), NMI, and NFATC3 were associated with cellular response to stress. Pathway maps for Pattern II showed that SMAD, JUND/AP1, and NFkB signaling are affected by 0.12 mg/L 2 nm Au-NPs at 2 hour. Pathway maps for Pattern III showed that many genes involved in RNA processing and DNA modification are changed at both 2 and 8 hours with 0.12 mg/L 20-40 nm Au-NP treatment. Pathway maps for Pattern N showed that MYC transcription regulation, and the protein folding-related heat-shock proteins are the major signaling pathways affected by 80-200 nm Au-NPs at both dosages at 2 hour.

### DETAILED DESCRIPTION

### I. Size Dependent Effect of Nanoparticles on Biological Organisms

In certain embodiments this invention pertains to the discovery that there are distinct and different molecular responses to exposure to nanoparticles of different sizes in a model cell system. Since nano-sized particulates have been present in the environment since the origin of life on Earth, other cell types or even organisms (prokaryotes or eukaryotes) are expected to share evolutionarily conserved cellular responses that are size-dependent and/or associated with other physico-chemical properties, such as shape and surface charge. Nanoparticles are increasingly used in consumer products and biomedical applications (Colvin et al. (2003) Nat. Biotech. 21: 1166-1170; Colvin (2004) Scientist 18: 26-27; Nel (2006) Science 311: 622-627). Yet relatively little is known about the molecular level cellular response to nanomaterials of different physico-chemical properties.

Gold nanoparticles (Au-NPs), one of the most commonly used nanoparticles in biotechnology (Jain (2005) Technol Cancer Res Treat 4: 645-650; Hirsch et al. (2006) Ann Biomed Eng 34: 15-22; Penn (2003) Curr Opin Chem Biol 7: 609-615; West and Halas (2003) Annu Rev Biomed Eng 5: 285-292), was used as a model system to help understand the size-dependent biological effect of nanoparticles. While Au-NPs are not completely inert in such a manner that would make them a perfect model system, their level of non-reactivity relative as compared to other nanoparticles permits the results reported here to serve as a close approximation for studying size-dependent effects of any nanoparticle.

Nanoparticles are used in a variety of industries that would benefit from understanding the effects of incorporating nanoparticles into their products or allowing them to be byproducts of their processes. More importantly, the recognition or identification of biological effects due simply to nanoparticle size rather than composition (e.g., chemical activity) allows product makers to determine whether or not adverse effects can be avoided simply by changing characteristic nanoparticle size or requires a change in the material composition or chemistry of the nanoparticle or formulation comprising the nanoparticles.

Thus, for example, where nanoparticles are used to deliver a pharmacological agent, it can be important to distinguish biological responses due solely to the size of the nanoparticles from the biological responses due to the nanoparticle material and/or the transported pharmaceutical.

Nanoparticles are used in a number of different industries and face similar concerns. For example, nanoparticles comprised of titanium dioxide are used in sunscreen, pearlescent nanoparticles are used in cosmetics. Nanoparticles are also used in organic waste or soil cleanup, as an aerated by product in paint and exhaust emissions (from burning carbon-based fuels and nanoparticles used as catalysts and cleanup in fuels), in the medicine field as used in therapeutics, wound repair, and various materials, and in pesticides and fertilizers whereby nanoparticles can be taken up by plants and subsequently enter the food supply.

Thus, determining the biological effects of nanoparticles is crucial to our understanding of how these particles may affect the world at large.

Using gold nanoparticles as a model system, size-dependent cell responses to the nanoparticles were identified that implicated multiple processes implicated, including, but not limited to signaling, intracellular compartmentalization and transportation, particle sorting and stress responses. We examined the global gene expression profiles of cells treated with Au-NPs to identify particle size-dependent molecular responses, using an Illumina microarray. Human Jurkat T lymphocytes were exposed to 1.2 mg/L or 0.12 mg/L of Au-NPs ranging from 2 nm to 200 nm in diameter, for either 2 or 8 hours (detail data in Supplement 1). Both size- and dose-dependent expression changes were observed.

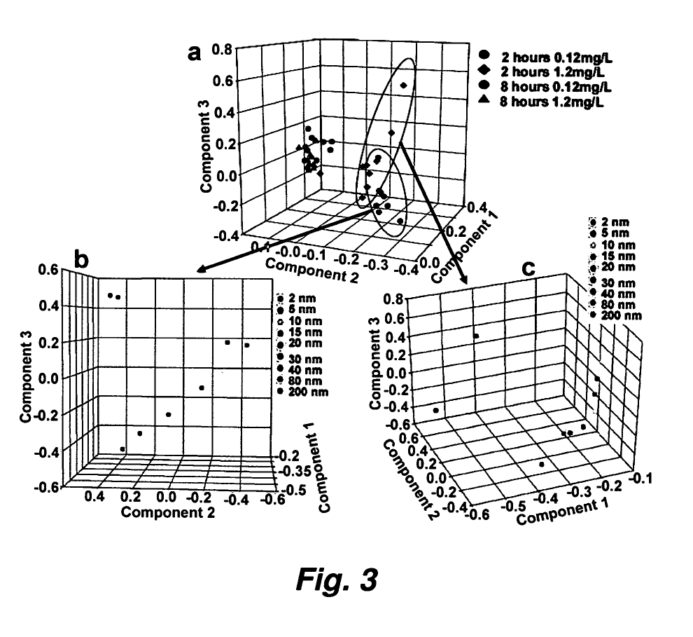

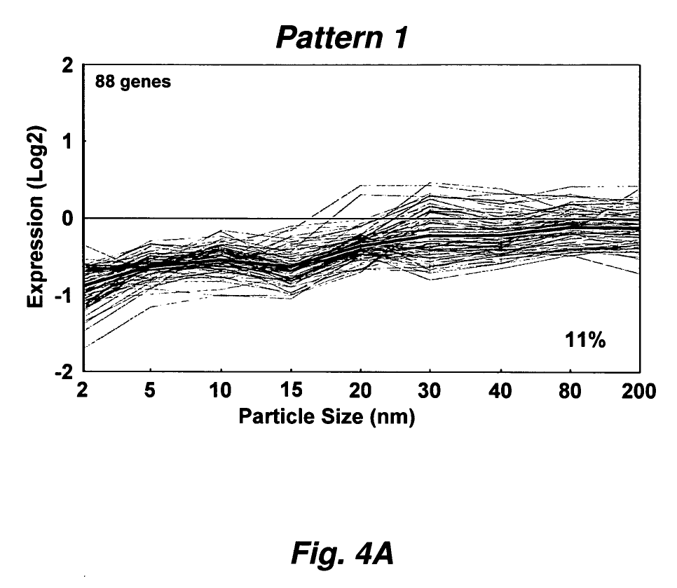

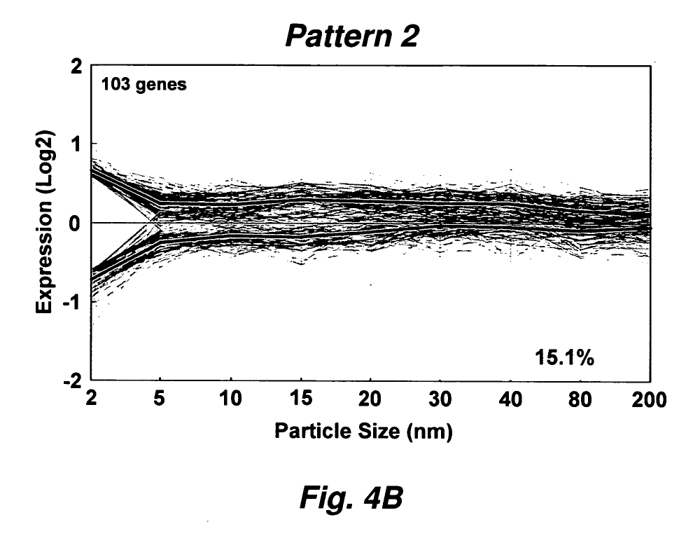

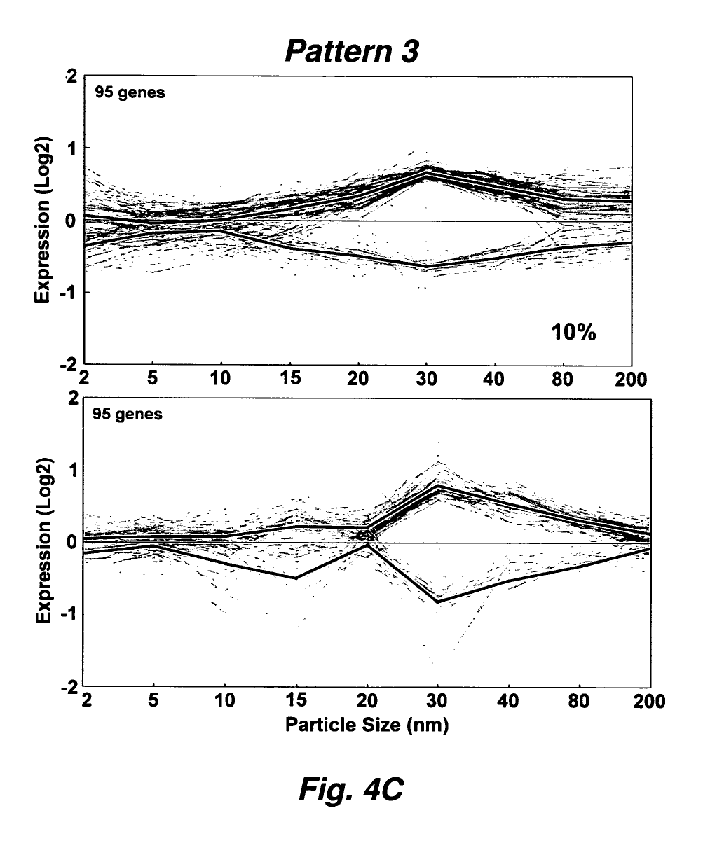

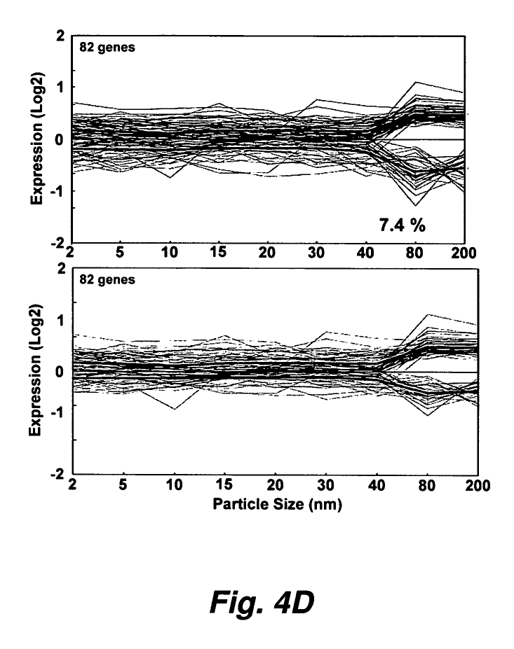

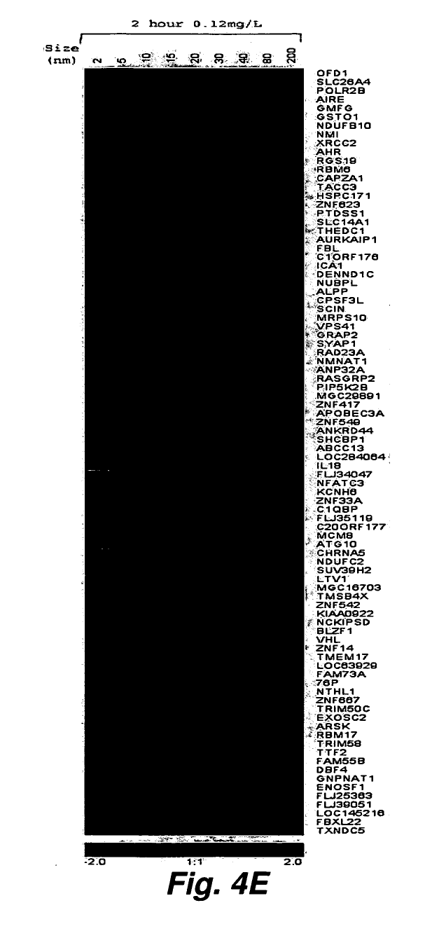

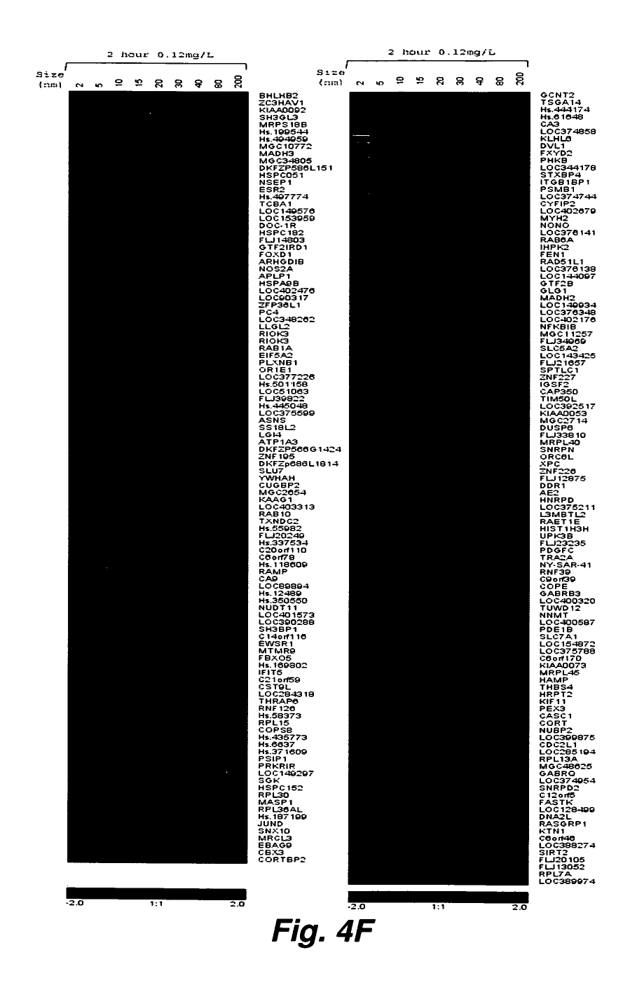

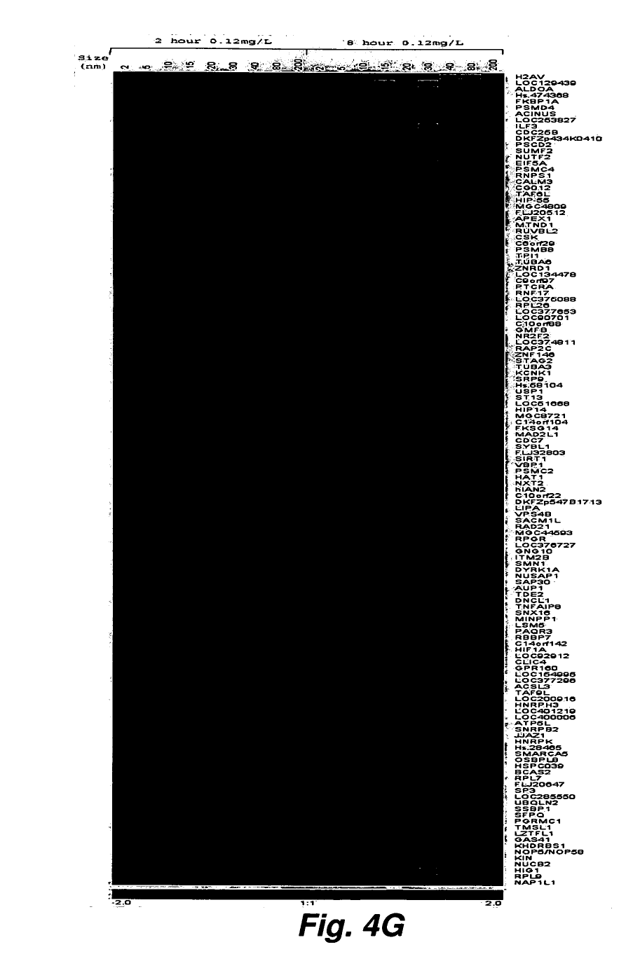

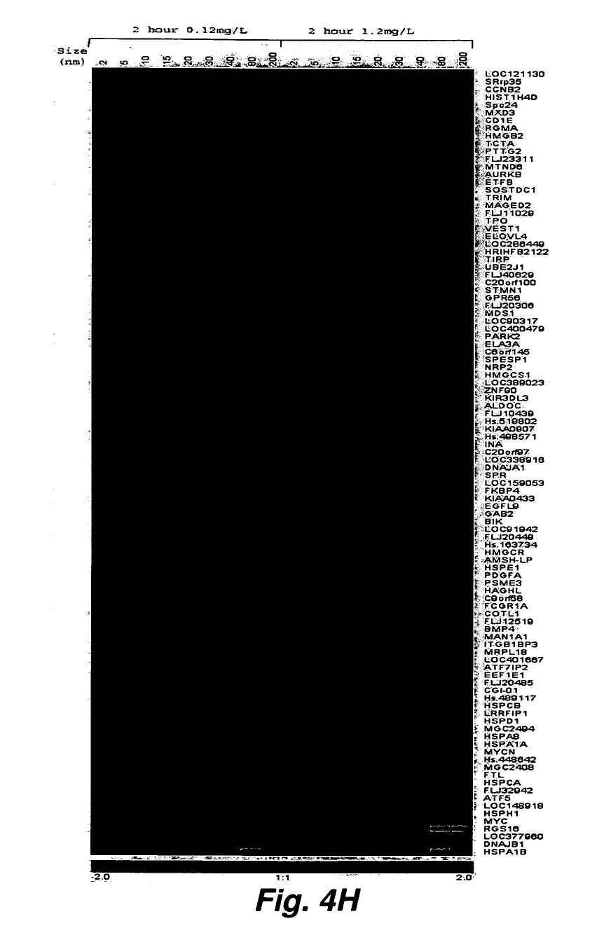

The 2 hour 0.12 mg/L dataset was focused on to illustrate the molecular mechanisms in finer detail because this treatment group showed the clearest size-dependent patterns (see, e.g., FIG. 3, panel b). Four dominant size-dependent gene expression patterns emerged from clustering analysis (see, e.g., FIGS. 4A-4D for size dependent expression patterns, and FIGS. 4E-4H for detailed gene lists and heatmaps). The genes comprising each of the four characteristic patterns are summarized as pattern set 1, pattern set 2, pattern set 3, and pattern set 4 in Table 1.

Pattern 1 (see FIGS. 4A and 4E as well as Table 2) produced by pattern set 1 genes, contains around 11% of the differentially expressed genes that show increased down-regulation in a pseudo-linear fashion when particle size decreases, with 40-80 nm as the upper limit. Without being bound to a particular theory, this is probably due to increased reactivity of the gold nanoparticles due to the increase in surface area when size decreases¹². However, the lack of an up-regulated linear pattern is in itself an intriguing phenomenon. These genes are functionally involved in stress response (e.g. IL18, NMI, NFATC3), DNA repair (e.g. RAD23A, XRCC2), transcription regulation, cytoskeleton organization and secretion (see, Table 1).

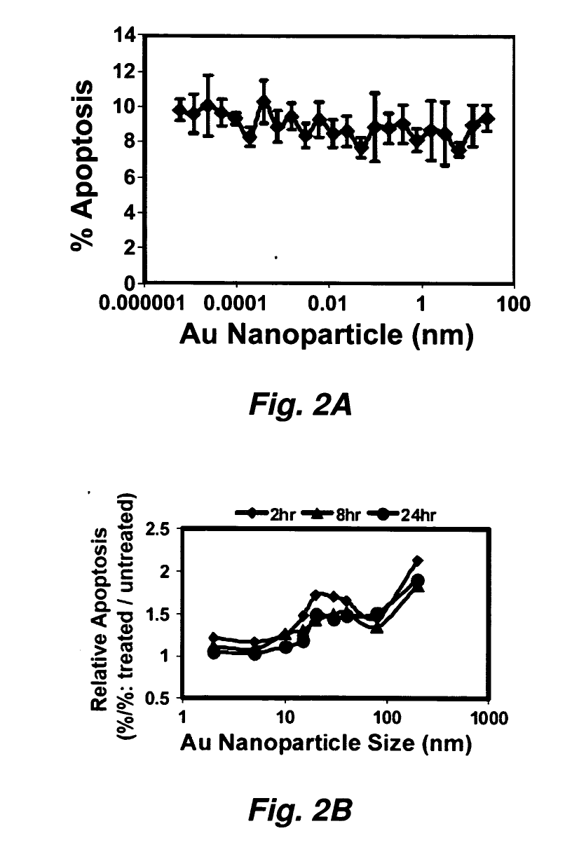

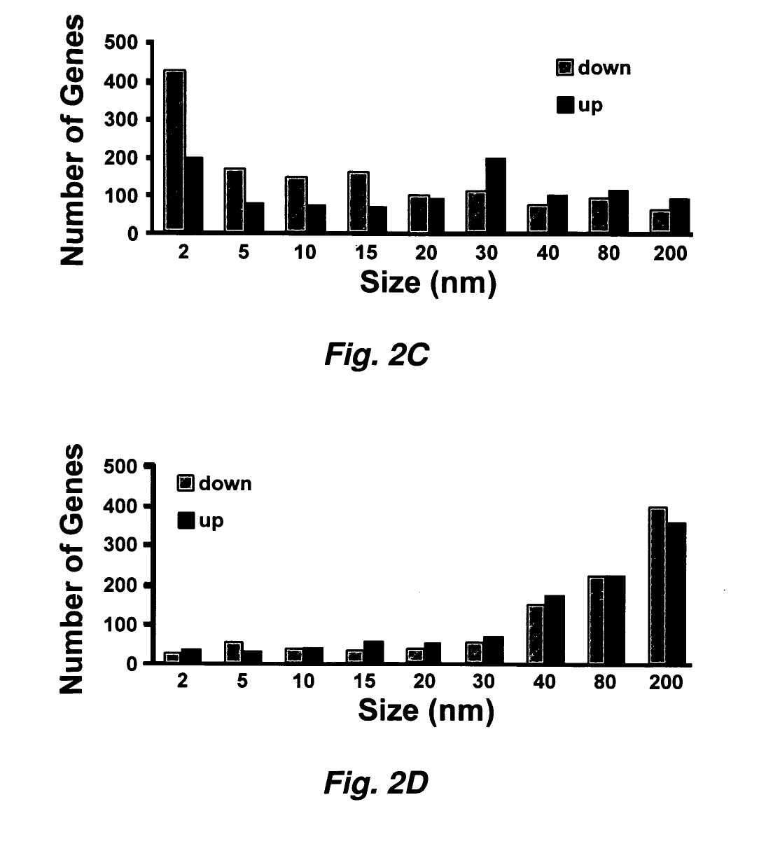

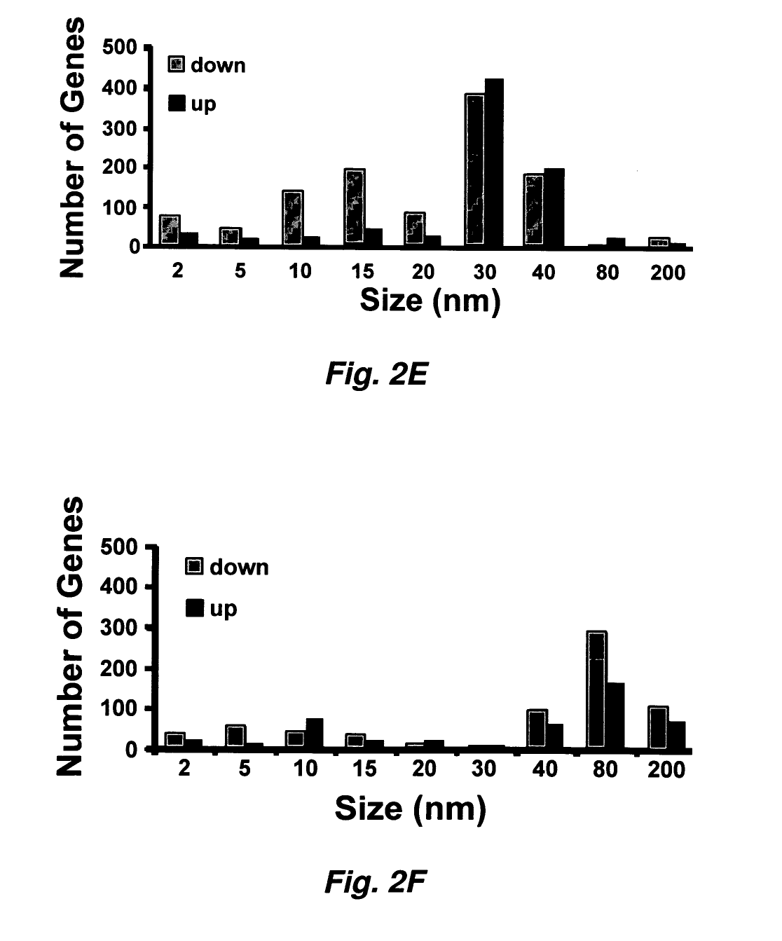

Pattern II (see FIGS. 4B and 4F, as well as Table 3) represents 15% of the genes and has altered expression for only the 2 nm treatment, which also reflects the observation of overall expression change (FIG. 2C). These genes are enriched in cellular functions and processes such as transcription (e.g. FOXD1, JUND, SMAD2, SMAD3), cell growth, cell signaling, apoptosis and response to virus (see Table 1).

Pattern III (see FIGS. 4C and 4G, as well as Table 4) shows 10% of the genes responding to the 20-40 nm Au-NPs at both the 2 hour and 8 hour time-points. These genes are involved in chromosome organization and packaging, DNA packaging, DNA repair, RNA metabolism, intracellular signaling and transcription (see Table 1). Pattern III persists over time and is the predominant expression pattern with the 0.12 mg/L Au-NP treatment at 8 hours (FIG. 2E); this pattern might be the underlying molecular signature for the preferential uptake of similar sized Au-NPs reported previously (Chithrani et al. (2006) Nano Lett 6: 662-668).

Pattern IV (see FIGS. 4D and 4H, as well as Table 5) consists of around 7.5% of the genes that are either down-regulated or up-regulated by treatment of larger Au-NPs (80-200 nm) in both dosage groups. Interestingly, genes in this group includes transcription factors such as MYC, MYCN, stress response genes, cell cycle genes and genes that are involved protein folding and transport (see Table 1). Pattern IV is dominant at the higher 1.2 mg/L dosage of the 2 hour time point as well, indicating that there might exist a physical barrier in the cell for nanoparticles larger than 80 nm.

TABLE 1
List of genes in pattern set 1, pattern set 2, pattern set 3, and pattern set 4.

| Pattern Set/function | Genes |
|---|---|
| Pattern Set 1 | |
| cytoskeleton organization | TMSB4X SCIN NCKIPSD CAPZA1 |
| and biogenesis | |
| transcription, DNA- | NFATC3, NMI, BLZF1, ZNF33A, AIRE, ZNF623, |
| dependent | POLR2B, VHL, TTF2, ZNF549, AHR, ZNF14, ZNF417, |
| | MCM8 |
| DNA repair | RAD23A, XRCC2, NTHL1 |
| Secretion | BLZF1, ICA1, |
| Response to stress | IL18, NFATC3, NMI, AIRE, RAD23A, VHL, GRAP2, |
| | XRCC2, NTHL1, AHR |
| Pattern Set 2 | |
| Transcription | BHLHB2, ESR2, FOXD1, GTF2B, GTF2IRD1, JUND, |
| | SIRT2, SMAD2, SMAD3, SUB1, YBX1, YWHAH |
| Growth | CA9, DVL1, EIFSA2, ESR2, EWSR1, JUND, NJKBIB, |
| | NOS2A, PCSK4, RAB1A, SMAD2, SMAD3, TRA2A, |
| | ZNF198 |
| Cell signaling | CEP57, DVL1, ESR2, FASTK, GABRB3, GABRQ, |
| | NFKBIB, NOS2A, PDE1B, PDGFC, PRKRIR, SH3GL3, |
| | SMAD2, SMAD3, STXBP4, YWHAH |
| activation of | GTF2B, JUND, SMAD3, SUB1, DUSP6, DVL1, NOS2A |
| virusApoptosis | |
| Transport of protein | HSPA9B, RAB10, RAB1A, RAB6A, YWHAH |
| Pattern Set 3 | |
| Chromosome organization | RAD21, GAS41, RUVBL2, JJAZ1, ACINUS, TAF6L, |
| and biogenesis | NAP1L1, H2AV, SMARCA5 |
| DNA repair | APEX1, RAD21, RUVBL2, USP1, SFPQ |
| DNA packaging | GAS41, RUVBL2, JJAZ1, TAF6L, NAP1L1, H2AV, |
| | HAT1, SMARCA5 |
| Intracellular signaling | CSK, HIP14, FKBP1A, LZTFL1, HIP-55, SNX16, |
| | RAP2C |
| RNA metabolism | LSM5, BCAS2, RNPS1, SFPQ, HNRPH3, NXT2, |
| | KHDRBS1, SNRPB2 |
| Transcription, DNA- | H1FA, ZNRD1, APEX1, GAS41, SAP30, RUVBL2, |
| dependent | JJAZ, ZNF146, TAF6L, SYBL1, SFPQ, NR2F2, VPS4B, |
| | KHDRBS1, ILF3, SMARCA5, SP3 |
| Pattern Set 4 | |
| Response to stress | DNAJB1, HSPDA, HSPAS, HMGB2, PTTG2, |
| | KIR3DL3, HSPH1, HSPCB, DNAJA1, HSPE1 |
| Organismal physiological | CDE, BMP4, ELA3A, ELOVL4, KIR3DL3, RGS16, |
| process | FCGR1A |
| Cell cycle | MYC, PDGFA, PTTG2, ATF5, CCNB2, AURKB |
| Response to unfolded | DNAJB1, HSPD1, HSPA8, HSPH1, HSPCB, DNAJA1, |
| protein | HSPE1 |
| transport | TIRP, HSPD1, FTL, ETFB, FCGR1A |
| Transcription. | C20orf97, MYC, MYCN, ZNF90, HMGB2, MXD3, |
| | PTTG2, ATF5, LRRFIP1 |

Using the patterns and pattern sets identified herein, one of skill can readily identify if a cell, tissue, or organism's response to nanoparticle is due simply to size-effects or other physio-chemical properties of the nanoparticulate.

Thus, for example, in certain embodiments, methods are provided for identifying size-dependent biological effects of a nanoparticle on a cell, where the methods involve contacting the cell with the nanoparticle(s) in question, and measuring levels of gene expression in said cell of at least two genes found in one or more of Pattern Set 1, Pattern Set 2, Pattern Set 3, or Pattern Set 4 (see, Table 1) where changes in expression level of the genes consistent with Pattern 1, Pattern 2, Pattern 3, or Pattern 4 indicate size effects of the nanoparticle(s) on the cell; and changes in expression level deviating from Pattern 1, Pattern 2, Pattern 3, and Pattern 4 is an indicator of biological effects that are not solely due to nanoparticle size.

In certain embodiments, methods are provided for identifying biological effects of a nanoparticle on a cell where the effects are not solely due to the size of said nanoparticle. The methods typically involve contacting the cell with the nanoparticle(s) in question, and measuring levels of gene expression in the cell wherein changes in expression level of genes other than genes found in one or more of Pattern Set 1, Pattern Set 2, Pattern Set 3, or Pattern Set 4, or changes of expression level of genes in one or more of Pattern Set 1, Pattern Set 2, Pattern Set 3, or Pattern Set 4 deviating from Pattern 1, Pattern 2, Pattern 3, and Pattern 4 is an indicator of biological effects that are not solely due to nanoparticle size.

The patterns of gene expression (patterns 1-4) are shown in FIGS. 4A-4D which also show average expression values (see lines in figures). In addition, level of expression for the various genes comprising each pattern set when the cell is contacted with nanoparticle sizes of 2 nm, 5 nm, 10 nm, 15 nm, 20 nm, 30 nm, 40 run, 80 nm, 200 nm are provided in Table 2 for pattern 1 (pattern set 1), Table 3 for pattern 2 (pattern set 2), Table 4 for pattern 3 (pattern set 3), and Table 5 for pattern 4 (pattern set 4). Using the Figures and/or the tables, expression levels of the various pattern set genes in patterns 1-4, can readily be interpolated for particle sizes between the 2 nm, 5 nm, 10 nm, 15 nm, 20 nm, 30 nm, 40 nm, 80 nm, and 200 nm values shown.

In certain embodiments changes in expression level of the measured genes (e.g., some or all of the genes listed in a pattern set) consistent with Pattern 1, Pattern 2, Pattern 3, or Pattern 4 indicates that the expression level at least 25%, preferably at least 50%, more preferably at least 75%, and most preferably at least 85%, 90%, 95%, or all of the measured genes is upregulated or downregulated in the same direction as the corresponding genes comprising the patters (e.g., as shown in Table 2 for pattern 1, Table 3 for pattern 2, Table 4 for pattern 3, or Table 5 for pattern 4). In other words, if exposure to the nanoparticle results in upregulation or downregulation of a plurality of the same genes in the same direction as shown in Patterns 1-4 (e.g., FIGS. 4A-4D, Tables 2-5, etc.) then for particles of approximately the same average size, then this result is an indicator that the biological effects of the tested nanoparticles is due to nanoparticle size rather than to properties of the nanoparticle.

In certain embodiments changes in expression level of the measured genes (e.g., some or all of the genes listed in a pattern set) consistent with Pattern 1, Pattern 2, Pattern 3, or Pattern 4 indicates that the magnitude of the expression level(s) of a plurality of the measured genes (e.g., at least 25%, preferably at least 50%, more preferably at least 75%, and most preferably at least 85%, 90%, 95%, or all of the measured genes) is comparable to (e.g., within 1 standard deviation (S.D.) preferably within 0.5 S.D., more preferably within 0.25 S.D., most preferably within 0.1 or 0.05 S.D.) the average magnitude shown in Pattern 1, Pattern 2, Pattern 3, or Pattern 4 for particles of the same size (e.g., as measured for the particle sizes shown, or as interpolated for other particle sizes from the data provided herein).

In certain embodiments changes in expression level of the measured genes (e.g., some or all of the genes listed in a pattern set) consistent with Pattern 1, Pattern 2, Pattern 3, or Pattern 4 indicates that there is no statistically significant difference (e.g., at better than a 10% confidence level, preferably at better than a 5% confidence level, more preferably at better than a 2% or 1% confidence level) in the expression level of the measured genes from the average expression levels comprising Pattern 1, pattern 2, pattern 3, or pattern 4. This can be determined using any appropriate statistical measure (e.g., t-test, analysis of variance, analysis of covariance, non-parametric test, etc.).

In certain embodiments method are also provided whereby the nanoparticle size is determined and compared against the present size toxicity standard(s) provided herein. The presently described size dependent biological effects can be used as a “gold” standard against which the toxicology of nanoparticles can be measured. In one embodiment, gene expression profiles and gene function, promoter and pathway analyses are performed for cells after exposure to the nanoparticle to be assessed and the patterns that emerge are compared to the presently described size-dependent patterns and genes shown in FIGS. 4A-4H. For example, in certain embodiments if there is a greater than 1.2-fold, preferably greater than a 1.5 fold, more preferably greater than a 2-fold or 5-fold increase or decrease in the expression of a particular gene corresponding to a particular size and pattern, more specific toxicology studies of the nanoparticle would be required.

In certain embodiments, the presently described patterns are used to screen out biological effects based on nanoparticle size, thus enabling the ability to study biological toxicological effects derived or driven by other aspects such as chemical make-up, shape, and surface of the nanoparticles.

In another embodiment, the present size-specific patterns and disclosed biomarkers enable one to design and engineer countermeasures to avoid specific effects. Also, the pattern sets provided herein effectively provide a listing of biomarkers that can be cited as unavoidably affected by the nanoparticles of specific sizes when filing for regulatory approval.

In another embodiment, using the biomarkers identified in the Tables that are associated with particular sized nanoparticles, it is possible to evaluate the cytotoxicity of various nanomaterials, using the biomarkers and biomarker temporal change patterns as predictors for other nanoparticles. It was found that particular biological pathways are activated or perturbed by nanoparticle size, these pathways and the nanoparticle specific biomarkers affect various cell processes, including stress response, DNA repair, apoptosis, chromosome organization and packaging, cell cycle, transport, etc. The changes in these biomarkers can be used as indicators or predictors for nanotoxicity.

TABLE 2
Genes screened for pattern set 1 (S3-T1-Pattern-I). Members of Pattern set 1 are
underlined.

| Gene | 2 nm | 5 nm | 10 nm | 15 nm | 20 nm | 30 nm | 40 nm | 80 nm | 200 nm |
|---|---|---|---|---|---|---|---|---|---|
| TMSB4X | −1.220 | −0.585 | −0.338 | −0.425 | 0.313 | 0.285 | 0.231 | 0.418 | 0.421 |
| XRCC2 | −1.107 | −0.635 | −0.425 | −0.646 | −0.293 | 0.302 | 0.194 | 0.319 | 0.152 |
| NOL5A | −0.444 | −0.562 | −0.513 | −0.675 | −0.571 | −0.482 | −0.359 | 0.317 | 0.435 |
| LOC34131 | −0.678 | −0.747 | −0.315 | −0.415 | −0.199 | 0.403 | 0.054 | 0.307 | 0.324 |
| CAPZA1 | −0.897 | −0.928 | −0.304 | −0.155 | 0.432 | 0.434 | 0.318 | 0.300 | 0.204 |
| VHL | −1.316 | −0.750 | −0.555 | −0.125 | −0.098 | 0.250 | 0.324 | 0.289 | 0.281 |
| NMI | −0.643 | −0.708 | −0.610 | −0.346 | −0.102 | 0.323 | 0.192 | 0.282 | 0.240 |
| LOC28512 | −0.752 | −0.430 | −0.987 | −0.616 | −0.643 | 0.069 | −0.088 | 0.254 | 0.191 |
| CCT6A | −0.768 | −0.652 | −0.488 | −0.332 | −0.317 | 0.106 | −0.046 | 0.249 | 0.227 |
| LOC38831 | −0.976 | −0.277 | −1.211 | −0.733 | −0.415 | 0.074 | −0.022 | 0.215 | 0.057 |
| FTH1 | −1.033 | −0.856 | −0.614 | −0.476 | −0.205 | 0.332 | 0.141 | 0.214 | 0.327 |
| TMEM17 | −0.700 | −0.676 | −0.636 | −0.674 | −0.378 | 0.168 | −0.037 | 0.203 | 0.066 |
| MAEA | −0.656 | −0.703 | −0.503 | −0.431 | 0.131 | 0.335 | 0.187 | 0.198 | 0.207 |
| BIRC4 | −0.335 | −0.536 | −0.541 | −0.598 | −0.348 | 0.304 | 0.115 | 0.194 | 0.110 |
| TXNDC5 | −0.716 | −0.424 | −0.677 | −0.365 | −0.247 | 0.085 | −0.021 | 0.193 | 0.197 |
| MGC16703 | −0.944 | −0.620 | −0.552 | −0.810 | −0.399 | 0.096 | −0.133 | 0.191 | 0.011 |
| FLJ21144 | −0.917 | −0.613 | −0.416 | −0.863 | −0.464 | 0.105 | 0.030 | 0.190 | 0.140 |
| EIF4G1 | −0.399 | −0.428 | −0.386 | −0.620 | −0.316 | −0.290 | −0.324 | 0.184 | 0.196 |
| ZNF549 | −0.675 | −0.670 | −0.622 | −0.597 | −0.699 | 0.074 | 0.008 | 0.183 | 0.082 |
| C1QBP | −1.064 | −0.986 | −0.926 | −0.749 | −0.671 | −0.050 | −0.138 | 0.178 | 0.217 |
| TTF2 | −0.629 | −0.591 | −0.563 | −0.633 | −0.293 | 0.093 | 0.079 | 0.147 | −0.015 |
| GSTO1 | −0.585 | −0.809 | −0.693 | −0.620 | −0.253 | 0.014 | 0.025 | 0.129 | 0.029 |
| NTHL1 | −0.674 | −0.626 | −0.381 | −0.668 | −0.465 | −0.476 | −0.380 | 0.127 | 0.119 |
| LOC34734 | −1.208 | −0.832 | −0.695 | −0.643 | −0.189 | −0.097 | −0.100 | 0.120 | 0.204 |
| LOC37752 | −0.891 | −0.621 | −0.403 | −0.122 | 0.188 | 0.500 | 0.373 | 0.119 | 0.179 |
| FBL | −0.655 | −0.679 | −0.560 | −0.431 | −0.206 | 0.196 | −0.048 | 0.110 | 0.063 |
| ZNF136 | −0.575 | −0.454 | −0.525 | −0.598 | −0.370 | −0.026 | −0.107 | 0.102 | −0.023 |
| LOC37675 | −0.662 | −0.658 | −0.227 | −0.697 | −0.223 | −0.158 | −0.162 | 0.099 | 0.175 |
| RBM17 | −0.758 | −0.534 | −0.625 | −0.688 | −0.213 | 0.468 | 0.387 | 0.094 | 0.128 |
| LOC377524 | −1.461 | −1.503 | −1.358 | −1.189 | −0.662 | 0.035 | −0.153 | 0.091 | 0.003 |
| EIF3S10 | −0.847 | −0.641 | −0.510 | −0.394 | −0.213 | 0.016 | −0.170 | 0.089 | 0.146 |
| VPS41 | −0.974 | −0.484 | −0.541 | −0.686 | −0.416 | −0.292 | −0.325 | 0.084 | −0.125 |
| LOC151194 | −0.431 | −0.485 | −0.440 | −0.670 | −0.248 | 0.304 | −0.003 | 0.081 | 0.150 |
| PTDSS1 | −1.034 | −0.731 | −0.535 | −0.394 | −0.302 | 0.156 | −0.021 | 0.067 | 0.053 |
| C6orf93 | −0.631 | −0.675 | −0.596 | −0.238 | −0.276 | 0.302 | 0.154 | 0.066 | 0.051 |
| KIAA0922 | −0.619 | −0.623 | −0.463 | −0.470 | −0.342 | −0.252 | −0.332 | 0.060 | 0.020 |
| PTP4A2 | −0.668 | −0.568 | −0.614 | −0.540 | 0.174 | 0.304 | 0.101 | 0.057 | 0.070 |
| LOC39997 | −0.882 | −0.600 | −0.626 | −0.650 | −0.310 | −0.113 | −0.136 | 0.052 | −0.026 |
| MSH3 | −0.771 | −0.964 | −0.604 | −0.876 | −0.496 | 0.224 | −0.004 | 0.043 | −0.144 |
| OFD1 | −0.704 | −0.617 | −0.360 | −0.102 | −0.025 | 0.289 | 0.234 | 0.032 | −0.007 |
| LOC13067 | −0.920 | −0.911 | −0.776 | −0.770 | −0.209 | −0.206 | −0.114 | 0.032 | 0.025 |
| Hs.153400 | −0.939 | −0.295 | −1.285 | −0.775 | −0.874 | −0.200 | −0.289 | 0.019 | 0.079 |
| POLR2H | −0.556 | −0.449 | −0.601 | −0.518 | −0.380 | −0.194 | −0.190 | 0.018 | 0.026 |
| BLZF1 | −0.669 | −0.335 | −0.462 | −0.586 | −0.213 | −0.140 | −0.297 | 0.006 | −0.065 |
| NCKIPSD | −0.774 | −0.687 | −0.581 | −0.609 | −0.277 | −0.198 | −0.273 | 0.002 | 0.219 |
| HPSE | −0.492 | −0.555 | −0.412 | −0.600 | −0.608 | 0.220 | 0.145 | −0.006 | −0.234 |
| CHRNA5 | −1.496 | −0.920 | −1.012 | −0.998 | −0.697 | −0.022 | −0.056 | −0.009 | −0.019 |
| BTBD5 | −1.039 | −0.617 | −0.667 | −0.709 | −0.151 | −0.268 | −0.171 | −0.011 | −0.157 |
| AKIP | −0.585 | −0.792 | −0.352 | −0.520 | −0.377 | −0.123 | −0.092 | −0.012 | −0.015 |
| NDUFB10 | −0.701 | −0.625 | −0.342 | −0.407 | −0.335 | 0.001 | −0.141 | −0.013 | 0.091 |
| GNPNAT1 | −0.603 | −0.647 | −0.489 | −0.786 | −0.473 | 0.078 | −0.073 | −0.019 | −0.013 |
| Hs.487099 | −0.647 | −0.080 | −0.924 | −0.577 | −0.429 | −0.111 | −0.265 | −0.021 | −0.130 |
| FLJ12078 | −0.393 | −0.387 | −0.474 | −0.728 | −0.273 | −0.051 | −0.023 | −0.025 | −0.053 |
| SMG1 | −1.313 | −0.691 | −0.630 | −0.838 | −0.555 | 0.050 | −0.046 | −0.026 | −0.166 |
| ZNF14 | −1.077 | −0.650 | −0.485 | −0.705 | −0.268 | −0.051 | −0.094 | −0.028 | −0.070 |
| NUP43 | −0.294 | −0.611 | −0.522 | −0.632 | −0.291 | −0.112 | −0.120 | −0.030 | −0.059 |
| BIA2 | −0.941 | −0.771 | −0.769 | −0.783 | −0.455 | 0.002 | −0.230 | −0.042 | −0.135 |
| LOC38914 | −0.840 | −0.658 | −0.328 | −0.250 | −0.082 | 0.036 | −0.057 | −0.043 | −0.050 |
| GOLGA2 | −1.212 | −0.790 | −0.625 | −0.826 | −0.202 | −0.104 | −0.144 | −0.043 | −0.076 |
| HSPC171 | −0.889 | −0.597 | −0.445 | −0.466 | −0.255 | −0.379 | −0.168 | −0.045 | 0.023 |
| TRIM50C | −0.620 | −0.338 | −0.367 | −0.633 | −0.315 | −0.176 | −0.179 | −0.047 | −0.063 |
| SH120 | −0.724 | −0.778 | −0.570 | −0.458 | −0.400 | 0.164 | 0.091 | −0.052 | 0.151 |
| LOC28440 | −1.004 | −0.321 | −1.036 | −0.986 | −0.667 | −0.428 | −0.257 | −0.055 | −0.086 |
| POLR2B | −0.691 | −0.560 | −0.661 | −0.302 | −0.111 | 0.121 | 0.127 | −0.057 | 0.002 |
| SLC26A4 | −1.000 | −0.589 | −0.547 | −0.653 | −0.402 | −0.145 | −0.143 | −0.061 | −0.084 |
| LOC37504 | −0.907 | −0.651 | −0.617 | −0.695 | −0.192 | −0.288 | −0.218 | −0.063 | −0.013 |
| NMNAT1 | −0.734 | −0.615 | −0.565 | −0.673 | −0.283 | −0.416 | −0.170 | −0.063 | −0.126 |
| FLJ11106 | −0.913 | −0.523 | −0.634 | −0.548 | −0.327 | −0.209 | −0.288 | −0.066 | −0.204 |
| LOC37687 | −0.789 | −0.781 | −0.503 | −0.427 | −0.162 | 0.113 | 0.219 | −0.069 | 0.088 |
| MRPS10 | −0.776 | −0.884 | −0.681 | −0.230 | −0.096 | 0.257 | 0.017 | −0.076 | −0.013 |
| LOC63929 | −0.691 | −0.588 | −0.532 | −0.494 | −0.254 | 0.109 | −0.088 | −0.076 | −0.100 |
| LOC284064 | −1.101 | −0.801 | −0.403 | −0.612 | −0.065 | 0.149 | 0.073 | −0.078 | 0.103 |
| LOC37459 | −1.119 | −0.818 | −0.660 | −0.755 | −0.319 | 0.030 | −0.101 | −0.079 | −0.035 |
| LOC284434 | −1.063 | −0.798 | −0.594 | −0.974 | −0.651 | −0.182 | −0.271 | −0.080 | −0.279 |
| FLJ35093 | −1.085 | −0.791 | −0.657 | −0.836 | −0.425 | −0.355 | −0.352 | −0.082 | −0.040 |
| KCNH6 | −1.008 | −0.804 | −0.699 | −0.815 | −0.339 | −0.134 | −0.122 | −0.088 | −0.150 |
| IL18 | −1.373 | −0.906 | −0.647 | −0.893 | −0.295 | −0.132 | −0.142 | −0.088 | −0.070 |
| ASK | −0.626 | −0.481 | −0.412 | −0.677 | −0.342 | −0.298 | −0.307 | −0.091 | −0.121 |
| MGC34079 | −1.202 | −0.577 | −0.595 | −0.844 | −0.521 | −0.394 | −0.497 | −0.094 | −0.138 |
| DKFZp727 | −0.807 | −0.383 | −0.489 | −0.618 | −0.351 | −0.139 | −0.253 | −0.095 | −0.036 |
| LOC34848 | −0.645 | −0.171 | −0.873 | −0.814 | −0.714 | −0.406 | −0.447 | −0.120 | −0.103 |
| LOC28617 | −0.945 | −0.632 | −0.398 | −0.688 | −0.430 | −0.273 | −0.357 | −0.122 | −0.080 |
| RBM6 | −0.530 | −0.605 | −0.779 | −0.486 | −0.387 | −0.380 | −0.132 | −0.124 | −0.205 |
| GRM7 | −0.182 | −0.943 | −0.242 | −0.754 | 0.031 | −0.215 | 0.156 | −0.126 | −0.161 |
| GMFG | −0.826 | −0.728 | −0.533 | −0.670 | −0.302 | 0.087 | 0.110 | −0.130 | −0.142 |
| LOC37614 | −0.691 | −0.820 | −0.505 | −0.598 | −0.263 | −0.157 | −0.146 | −0.130 | −0.002 |
| LOC28380 | −0.757 | −0.505 | −0.304 | −0.650 | −0.199 | −0.157 | −0.212 | −0.132 | −0.244 |
| RPL8 | −0.893 | −0.756 | −0.487 | −0.685 | −0.650 | −0.259 | −0.164 | −0.140 | 0.180 |
| LOC343384 | −1.215 | −1.217 | −0.756 | −0.963 | −0.569 | −0.156 | −0.186 | −0.143 | 0.019 |
| LOC37667 | −1.087 | −0.720 | −0.495 | −0.712 | −0.313 | −0.301 | −0.262 | −0.145 | −0.173 |
| LOC37795 | −0.823 | −0.545 | −0.585 | −0.596 | −0.479 | −0.248 | −0.306 | −0.145 | −0.070 |
| FAM31C | −0.995 | −0.607 | −0.354 | −0.516 | −0.239 | −0.416 | −0.354 | −0.146 | −0.231 |
| HSRTSBET | −0.645 | −0.664 | −0.804 | −0.561 | −0.515 | −0.023 | −0.145 | −0.156 | −0.126 |
| FLJ34278 | −0.686 | −0.403 | −0.502 | −0.668 | −0.371 | −0.081 | −0.299 | −0.156 | −0.222 |
| GPS2 | −0.453 | −0.506 | −0.748 | −0.489 | −0.079 | −0.327 | −0.397 | −0.159 | 0.129 |
| ALPP | −0.809 | −0.459 | −0.433 | −0.807 | −0.392 | −0.297 | −0.308 | −0.169 | −0.215 |
| FLJ34047 | −1.725 | −1.158 | −0.995 | −1.044 | −0.477 | −0.337 | −0.375 | −0.176 | −0.132 |
| ANP32A | −0.911 | −0.490 | −0.594 | −0.328 | −0.418 | −0.368 | −0.249 | −0.177 | 0.398 |
| MBNL1 | −0.879 | −0.890 | −0.567 | −0.508 | −0.446 | 0.074 | −0.189 | −0.180 | −0.133 |
| SYAP1 | −0.773 | −0.557 | −0.425 | −0.674 | −0.393 | −0.269 | −0.248 | −0.181 | −0.204 |
| ABCC13 | −0.970 | −0.650 | −0.676 | −0.850 | −0.479 | −0.359 | −0.318 | −0.193 | −0.204 |
| PIP5K2B | −1.191 | −0.466 | −0.430 | −0.618 | −0.337 | −0.406 | −0.370 | −0.200 | −0.307 |
| AHR | −0.647 | −0.647 | −0.655 | −0.682 | −0.225 | −0.211 | −0.225 | −0.202 | −0.139 |
| NFATC3 | −0.602 | −0.639 | −0.568 | −0.785 | −0.385 | −0.507 | −0.255 | −0.203 | −0.145 |
| C20orf177 | −0.995 | −0.661 | −0.575 | −0.666 | −0.401 | −0.326 | −0.304 | −0.204 | −0.241 |
| LOC375834 | −0.688 | −0.598 | −0.492 | −0.380 | −0.511 | −0.441 | −0.376 | −0.217 | −0.156 |
| NARF | −0.745 | −0.628 | −0.436 | −0.571 | −0.384 | −0.279 | −0.253 | −0.220 | −0.242 |
| LOC38793 | −0.660 | −0.424 | −1.108 | −0.486 | −0.528 | 0.201 | 0.056 | −0.222 | −0.170 |
| ZNF542 | −1.157 | −0.497 | −0.722 | −0.588 | −0.541 | −0.457 | −0.465 | −0.227 | −0.234 |
| UQCRH | −0.821 | −0.591 | −0.353 | −0.312 | −0.153 | 0.094 | 0.139 | −0.228 | 0.024 |
| APOBEC3A | −0.993 | −0.644 | −0.391 | −0.775 | −0.430 | −0.379 | −0.461 | −0.236 | −0.341 |
| MGC29891 | −0.852 | −0.517 | −0.422 | −0.751 | −0.422 | −0.560 | −0.379 | −0.252 | −0.309 |
| LOC51145 | −1.099 | −0.803 | −0.629 | −1.029 | −0.587 | −0.438 | −0.382 | −0.260 | −0.169 |
| LOC40148 | −1.223 | −0.559 | −0.612 | −0.616 | −0.435 | −0.378 | −0.255 | −0.261 | −0.187 |
| 76P | −1.084 | −0.531 | −0.625 | −0.716 | −0.273 | −0.540 | −0.432 | −0.262 | −0.157 |
| FLJ13815 | −1.009 | −0.333 | −0.333 | −0.630 | −0.312 | −0.481 | −0.445 | −0.272 | −0.283 |
| LOC37797 | −1.099 | −0.632 | −0.693 | −0.539 | −0.365 | −0.361 | −0.187 | −0.291 | −0.120 |
| LOC34422 | −0.790 | −0.766 | −0.351 | −0.576 | −0.460 | −0.401 | −0.530 | −0.297 | −0.131 |
| RAD23A | −0.704 | −0.575 | −0.624 | −0.653 | −0.587 | −0.320 | −0.417 | −0.304 | −0.373 |
| LOC37521 | −0.742 | −0.405 | −0.344 | −0.584 | −0.478 | −0.377 | −0.483 | −0.311 | −0.056 |
| C6orf29 | −0.757 | −0.436 | −0.307 | −0.698 | −0.385 | −0.590 | −0.422 | −0.311 | −0.385 |
| FLJ25224 | −1.148 | −0.719 | −0.579 | −0.771 | −0.359 | −0.518 | −0.407 | −0.311 | −0.154 |
| KIAA1473 | −1.164 | −0.390 | −0.485 | −0.634 | −0.282 | −0.480 | −0.449 | −0.319 | −0.328 |
| SMAP-1 | −1.098 | −0.497 | −0.379 | −0.734 | −0.595 | −0.497 | −0.412 | −0.323 | −0.379 |
| KIAA0628 | −1.400 | −0.834 | −0.682 | −0.822 | −0.395 | −0.591 | −0.451 | −0.335 | −0.316 |
| SCNM1 | −0.791 | −0.640 | −0.487 | −0.706 | −0.413 | −0.409 | −0.223 | −0.339 | −0.215 |
| NDUFC2 | −0.801 | −0.570 | −0.670 | −0.691 | −0.307 | −0.407 | −0.340 | −0.348 | −0.289 |
| APG10L | −1.100 | −0.356 | −0.383 | −0.600 | −0.298 | −0.444 | −0.330 | −0.356 | −0.430 |
| SHCBP1 | −0.922 | −0.593 | −0.400 | −0.632 | −0.460 | −0.618 | −0.555 | −0.364 | −0.268 |
| LOC37593 | −0.989 | −0.969 | −0.590 | −0.706 | −0.749 | −0.594 | −0.513 | −0.381 | −0.446 |
| LOC91526 | −1.180 | −0.813 | −0.754 | −0.987 | −0.659 | −0.683 | −0.579 | −0.385 | −0.385 |
| GRAP2 | −0.777 | −0.814 | −0.485 | −0.550 | −0.530 | −0.333 | −0.358 | −0.388 | −0.363 |
| KIAA0924 | −1.205 | −0.698 | −0.496 | −0.943 | −0.638 | −0.563 | −0.393 | −0.393 | −0.314 |
| AIRE | −1.152 | −0.550 | −0.471 | −0.804 | −0.660 | −0.626 | −0.471 | −0.397 | −0.340 |
| MCM8 | −1.215 | −0.646 | −0.476 | −0.807 | −0.535 | −0.624 | −0.476 | −0.399 | −0.389 |
| LOC402694 | −0.983 | −0.871 | −0.799 | −0.488 | −0.472 | −0.142 | −0.106 | −0.401 | −0.359 |
| SCIN | −0.981 | −0.614 | −0.507 | −0.682 | −0.417 | −0.663 | −0.537 | −0.403 | −0.388 |
| FLJ35119 | −1.048 | −0.827 | −0.819 | −0.961 | −0.602 | −0.402 | −0.480 | −0.404 | −0.309 |
| IPLA2(GAM | −1.124 | −0.348 | −0.296 | −0.659 | −0.250 | −0.620 | −0.462 | −0.409 | −0.314 |
| ZNF33A | −0.902 | −0.495 | −0.526 | −0.786 | −0.425 | −0.611 | −0.484 | −0.409 | −0.306 |
| FLJ14011 | −1.176 | −0.736 | −0.603 | −0.820 | −0.404 | −0.641 | −0.442 | −0.427 | −0.330 |
| LOC25382 | −0.721 | −0.613 | −0.453 | −0.768 | −0.521 | −0.697 | −0.622 | −0.428 | −0.464 |
| SUV39H2 | −1.158 | −0.672 | −0.772 | −0.893 | −0.651 | −0.423 | −0.392 | −0.428 | −0.431 |
| C14orf127 | −0.949 | −0.671 | −0.538 | −0.978 | −0.495 | −0.710 | −0.587 | −0.437 | −0.434 |
| DKFZp313 | −1.247 | −0.599 | −0.497 | −0.646 | −0.457 | −0.567 | −0.520 | −0.445 | −0.331 |
| FLJ40113 | −0.728 | −0.658 | −0.618 | −0.623 | −0.289 | −0.439 | −0.424 | −0.454 | −0.471 |
| PTTG3 | −0.673 | −0.861 | −0.636 | −0.511 | −0.188 | −0.034 | −0.219 | −0.457 | −0.320 |
| MGC44287 | −1.104 | −0.553 | −0.595 | −0.967 | −0.584 | −0.731 | −0.631 | −0.469 | −0.485 |
| LOC40108 | −0.852 | −0.525 | −0.447 | −0.679 | −0.211 | −0.521 | −0.395 | −0.482 | −0.365 |
| ICA1 | −0.855 | −0.451 | −0.525 | −0.691 | −0.329 | −0.552 | −0.414 | −0.484 | −0.328 |
| FLJ20006 | −1.395 | −0.861 | −0.693 | −1.020 | −0.590 | −0.666 | −0.449 | −0.499 | −0.310 |
| SLC7A11 | −0.610 | −0.631 | −0.551 | −0.706 | −0.364 | −0.512 | −0.579 | −0.542 | −0.355 |
| LOC255374 | −1.459 | −0.893 | −0.757 | −1.028 | −0.685 | −0.623 | −0.639 | −0.546 | −0.473 |
| LOC92755 | −1.167 | −1.088 | −0.823 | −1.099 | −0.595 | −0.277 | −0.301 | −0.546 | −0.345 |
| ZNF339 | −1.310 | −0.900 | −0.858 | −0.954 | −0.690 | −0.586 | −0.586 | −0.547 | −0.442 |
| FLJ21673 | −1.128 | −0.654 | −0.634 | −0.825 | −0.536 | −0.757 | −0.652 | −0.547 | −0.417 |
| MICAL3 | −1.263 | −0.582 | −0.758 | −1.030 | −0.515 | −0.673 | −0.515 | −0.577 | −0.436 |

TABLE 3
Genes screened for pattern set 2 (S3-T2-Pattern-II).

| Gene | 2 nm | 5 nm | 10 nm | 15 nm | 20 nm | 30 nm | 40 nm | 80 nm | 200 nm |
|---|---|---|---|---|---|---|---|---|---|
| BHLHB2 | 0.585 | 0.236 | 0.483 | 0.386 | 0.282 | 0.325 | 0.463 | 0.244 | 0.323 |
| ZC3HAV1 | 0.585 | 0.045 | 0.092 | 0.249 | 0.315 | 0.297 | 0.222 | −0.362 | −0.403 |
| KIAA0092 | 0.586 | 0.285 | 0.249 | 0.329 | 0.223 | 0.085 | 0.159 | 0.199 | 0.061 |
| SH3GL3 | 0.586 | 0.158 | 0.080 | 0.252 | 0.218 | 0.287 | 0.321 | 0.008 | 0.032 |
| MRPS18B | 0.588 | 0.256 | 0.190 | 0.430 | 0.461 | 0.451 | 0.433 | 0.315 | 0.258 |
| Hs.199544 | 0.589 | 0.098 | 0.176 | 0.431 | 0.144 | 0.334 | 0.389 | 0.180 | 0.069 |
| Hs.494959 | 0.592 | 0.342 | 0.280 | 0.413 | 0.395 | 0.180 | 0.366 | 0.295 | 0.174 |
| MGC10772 | 0.592 | 0.289 | 0.345 | 0.236 | 0.203 | −0.121 | 0.091 | 0.331 | 0.329 |
| MADH3 | 0.594 | 0.323 | 0.452 | 0.322 | 0.342 | 0.246 | 0.296 | 0.234 | 0.227 |
| MGC34805 | 0.595 | 0.347 | 0.251 | 0.254 | 0.182 | 0.214 | 0.239 | −0.097 | 0.016 |
| DKFZP586 | 0.595 | 0.259 | 0.447 | 0.532 | 0.480 | 0.312 | 0.328 | 0.094 | 0.241 |
| HSPC051 | 0.596 | 0.199 | 0.173 | 0.132 | 0.294 | −0.057 | 0.173 | 0.145 | 0.151 |
| NSEP1 | 0.596 | 0.229 | 0.130 | 0.407 | 0.329 | 0.475 | 0.191 | 0.344 | 0.294 |
| ESR2 | 0.596 | 0.494 | 0.059 | 0.366 | 0.275 | 0.440 | 0.369 | 0.582 | −0.114 |
| Hs.497774 | 0.597 | −0.040 | 0.140 | −0.050 | 0.096 | 0.044 | 0.166 | 0.079 | −0.022 |
| TCBA1 | 0.598 | −0.036 | 0.319 | 0.232 | 0.139 | 0.071 | −0.107 | 0.299 | 0.118 |
| LOC14957 | 0.599 | 0.314 | 0.301 | 0.281 | 0.223 | 0.276 | 0.370 | −0.262 | −0.205 |
| LOC15395 | 0.599 | 0.180 | 0.086 | 0.322 | −0.042 | 0.125 | 0.412 | 0.154 | 0.086 |
| DOC-1R | 0.600 | 0.415 | 0.539 | 0.401 | 0.138 | 0.199 | 0.237 | 0.032 | 0.031 |
| HSPC182 | 0.601 | 0.309 | 0.339 | 0.311 | 0.377 | 0.027 | 0.094 | −0.040 | 0.068 |
| FLJ14803 | 0.603 | 0.266 | 0.253 | 0.427 | 0.367 | 0.389 | 0.292 | −0.116 | −0.044 |
| GTF2IRD1 | 0.603 | 0.083 | −0.017 | 0.146 | 0.354 | 0.189 | 0.159 | 0.014 | 0.168 |
| FOXD1 | 0.604 | 0.291 | 0.293 | 0.497 | 0.416 | 0.294 | 0.496 | 0.160 | 0.164 |
| ARHGDIB | 0.606 | 0.089 | 0.108 | 0.066 | 0.148 | −0.064 | 0.019 | −0.081 | −0.064 |
| NOS2A | 0.609 | 0.349 | 0.491 | 0.296 | 0.412 | 0.290 | 0.320 | 0.201 | 0.237 |
| APLP1 | 0.611 | 0.191 | 0.095 | 0.068 | 0.368 | 0.160 | 0.089 | 0.066 | 0.063 |
| HSPA9B | 0.611 | 0.372 | 0.324 | 0.413 | 0.181 | 0.137 | 0.205 | 0.320 | 0.320 |
| LOC40247 | 0.612 | −0.003 | 0.188 | 0.422 | 0.224 | 0.379 | 0.414 | 0.305 | 0.447 |
| LOC90317 | 0.613 | 0.223 | 0.252 | 0.296 | 0.198 | 0.090 | 0.002 | 0.275 | 0.229 |
| ZFP36L1 | 0.613 | 0.414 | 0.246 | 0.519 | 0.412 | 0.422 | 0.348 | 0.214 | 0.108 |
| PC4 | 0.616 | 0.164 | 0.367 | 0.397 | 0.356 | 0.364 | 0.326 | 0.290 | 0.241 |
| LOC34826 | 0.621 | 0.557 | 0.286 | 0.390 | 0.185 | 0.003 | 0.024 | 0.024 | 0.075 |
| LLGL2 | 0.621 | −0.005 | 0.153 | 0.305 | 0.043 | 0.024 | 0.008 | 0.279 | 0.091 |
| RIOK3 | 0.622 | 0.423 | 0.271 | 0.447 | 0.252 | 0.402 | 0.395 | 0.243 | 0.196 |
| RIOK3 | 0.515 | 0.161 | 0.040 | 0.514 | 0.153 | 0.217 | 0.686 | 0.456 | 0.231 |
| RAB1A | 0.623 | 0.156 | 0.376 | 0.493 | 0.358 | 0.345 | 0.267 | 0.274 | 0.242 |
| EIF5A2 | 0.627 | 0.334 | 0.060 | 0.062 | 0.143 | 0.034 | 0.021 | −0.119 | −0.238 |
| PLXNB1 | 0.627 | 0.054 | 0.402 | 0.261 | 0.256 | 0.114 | 0.070 | 0.183 | 0.280 |
| OR1E1 | 0.628 | 0.576 | 0.007 | 0.413 | 0.556 | 0.035 | 0.166 | 0.156 | 0.309 |
| LOC37722 | 0.629 | 0.123 | 0.235 | 0.054 | 0.262 | 0.147 | 0.248 | 0.226 | 0.068 |
| Hs.501158 | 0.630 | −0.025 | 0.196 | 0.311 | 0.219 | 0.266 | 0.140 | −0.047 | 0.147 |
| LOC51063 | 0.631 | 0.193 | 0.313 | 0.012 | 0.299 | 0.230 | 0.372 | 0.080 | 0.258 |
| FLJ39822 | 0.632 | 0.297 | 0.467 | 0.436 | 0.635 | 0.395 | 0.371 | 0.023 | 0.252 |
| Hs.445048 | 0.632 | 0.350 | 0.350 | 0.201 | 0.364 | 0.267 | 0.242 | 0.129 | 0.061 |
| LOC37559 | 0.633 | 0.493 | 0.161 | 0.328 | 0.215 | −0.222 | 0.154 | 0.009 | 0.344 |
| ASNS | 0.634 | 0.277 | 0.299 | 0.256 | 0.281 | 0.253 | 0.315 | −0.057 | 0.103 |
| SS18L2 | 0.636 | 0.192 | 0.187 | 0.350 | 0.352 | 0.162 | 0.254 | 0.156 | 0.082 |
| LGI4 | 0.636 | 0.192 | 0.259 | 0.202 | 0.122 | 0.174 | 0.115 | 0.462 | 0.276 |
| ATP1A3 | 0.638 | 0.249 | 0.389 | 0.285 | 0.354 | 0.199 | 0.203 | 0.115 | 0.161 |
| DKFZP566 | 0.638 | 0.316 | 0.217 | 0.256 | 0.184 | 0.204 | 0.190 | 0.137 | 0.123 |
| ZNF195 | 0.640 | 0.117 | 0.202 | 0.232 | 0.409 | 0.443 | 0.309 | −0.028 | 0.019 |
| DKFZp686 | 0.640 | 0.281 | 0.162 | −0.005 | 0.265 | 0.213 | 0.279 | −0.328 | −0.083 |
| SLU7 | 0.642 | 0.025 | 0.104 | 0.379 | 0.310 | 0.351 | 0.388 | 0.191 | 0.288 |
| YWHAH | 0.642 | 0.147 | 0.160 | 0.208 | 0.127 | 0.293 | 0.237 | 0.322 | 0.203 |
| CUGBP2 | 0.650 | 0.388 | 0.359 | 0.514 | 0.442 | 0.518 | 0.365 | 0.023 | −0.012 |
| MGC2654 | 0.650 | 0.205 | 0.175 | 0.212 | 0.196 | 0.062 | 0.249 | −0.022 | −0.231 |
| KAAG1 | 0.651 | 0.145 | −0.075 | −0.140 | 0.152 | −0.104 | −0.024 | 0.226 | 0.111 |
| LOC40331 | 0.651 | 0.205 | 0.118 | 0.290 | 0.256 | 0.062 | 0.218 | −0.019 | −0.018 |
| RAB10 | 0.653 | 0.092 | 0.155 | 0.226 | 0.380 | 0.192 | 0.315 | 0.389 | 0.351 |
| TXNDC2 | 0.654 | 0.355 | 0.230 | 0.161 | 0.049 | 0.219 | 0.084 | 0.027 | −0.006 |
| Hs.55982 | 0.655 | 0.236 | 0.364 | 0.479 | 0.362 | 0.387 | 0.237 | 0.396 | 0.330 |
| FLJ20249 | 0.655 | 0.165 | 0.071 | 0.280 | 0.203 | 0.226 | 0.204 | 0.220 | 0.316 |
| Hs.337534 | 0.656 | 0.351 | 0.104 | 0.111 | 0.000 | 0.183 | 0.147 | 0.008 | −0.036 |
| C20orf110 | 0.658 | 0.369 | 0.374 | 0.348 | 0.289 | 0.276 | 0.321 | 0.196 | 0.099 |
| C6orf78 | 0.658 | 0.259 | 0.293 | 0.554 | 0.472 | 0.347 | 0.315 | 0.303 | 0.262 |
| Hs.118609 | 0.663 | 0.063 | 0.078 | 0.131 | 0.202 | 0.191 | 0.144 | 0.216 | 0.226 |
| RAMP | 0.663 | 0.182 | 0.224 | 0.262 | 0.258 | 0.322 | 0.379 | 0.117 | 0.239 |
| CA9 | 0.664 | 0.124 | −0.076 | 0.081 | 0.237 | 0.215 | 0.110 | 0.359 | 0.218 |
| LOC89894 | 0.664 | 0.336 | 0.344 | 0.516 | 0.303 | 0.482 | 0.528 | 0.009 | 0.012 |
| Hs.12489 | 0.664 | 0.427 | −0.111 | 0.280 | 0.325 | 0.257 | 0.202 | 0.230 | 0.162 |
| Hs.350550 | 0.665 | 0.521 | 0.270 | 0.338 | 0.105 | 0.360 | 0.285 | 0.339 | 0.412 |
| NUDT11 | 0.665 | 0.110 | 0.242 | 0.357 | 0.369 | 0.491 | 0.455 | 0.193 | 0.184 |
| LOC40157 | 0.667 | 0.348 | 0.464 | 0.272 | 0.253 | 0.410 | 0.387 | 0.214 | 0.217 |
| LOC39028 | 0.671 | 0.276 | 0.219 | 0.490 | 0.140 | 0.430 | 0.309 | 0.242 | 0.189 |
| SH3BP1 | 0.672 | 0.262 | 0.270 | 0.275 | 0.409 | 0.024 | 0.159 | 0.320 | −0.017 |
| C14orf116 | 0.679 | 0.109 | 0.017 | 0.132 | 0.284 | 0.371 | 0.224 | 0.174 | 0.029 |
| EWSR1 | 0.679 | 0.320 | 0.157 | 0.252 | 0.297 | 0.310 | 0.144 | 0.254 | 0.193 |
| MTMR9 | 0.680 | 0.255 | 0.419 | 0.471 | 0.396 | 0.355 | 0.332 | 0.122 | 0.102 |
| FBXO5 | 0.680 | 0.220 | 0.207 | 0.286 | 0.372 | 0.441 | 0.344 | −0.078 | 0.052 |
| Hs.169802 | 0.680 | 0.349 | 0.046 | 0.245 | 0.362 | 0.375 | 0.245 | 0.370 | 0.365 |
| IFIT5 | 0.681 | 0.301 | 0.056 | 0.261 | 0.267 | 0.450 | 0.307 | 0.410 | 0.339 |
| C21orf59 | 0.681 | 0.353 | 0.389 | 0.273 | 0.421 | 0.063 | 0.200 | 0.035 | 0.112 |
| CST9L | 0.683 | −0.086 | 0.082 | 0.314 | 0.295 | 0.133 | 0.145 | 0.328 | 0.207 |
| LOC28431 | 0.686 | −0.074 | 0.312 | 0.131 | 0.472 | 0.209 | 0.358 | 0.143 | 0.111 |
| THRAP6 | 0.686 | 0.274 | 0.358 | 0.507 | 0.335 | 0.393 | 0.290 | 0.048 | 0.090 |
| RNF126 | 0.690 | 0.325 | 0.378 | 0.510 | 0.439 | 0.134 | 0.154 | 0.037 | 0.099 |
| Hs.58373 | 0.691 | 0.475 | 0.201 | 0.351 | 0.242 | 0.295 | 0.330 | 0.032 | 0.154 |
| RPL15 | 0.697 | 0.320 | 0.239 | 0.324 | 0.176 | 0.176 | 0.061 | 0.042 | 0.117 |
| COPS8 | 0.698 | 0.224 | 0.108 | 0.362 | 0.391 | 0.381 | 0.334 | 0.275 | 0.338 |
| Hs.435773 | 0.700 | 0.360 | 0.224 | 0.389 | 0.210 | 0.374 | 0.356 | 0.282 | 0.240 |
| Hs.6637 | 0.703 | 0.308 | 0.228 | 0.254 | 0.320 | 0.323 | 0.220 | 0.234 | 0.128 |
| Hs.371609 | 0.707 | 0.377 | 0.167 | 0.279 | 0.232 | 0.316 | 0.216 | 0.067 | 0.004 |
| PSIP1 | 0.707 | 0.119 | 0.128 | 0.364 | 0.287 | 0.611 | 0.354 | 0.036 | 0.013 |
| PRKRIR | 0.714 | 0.295 | 0.307 | 0.274 | 0.371 | 0.367 | 0.263 | 0.028 | 0.078 |
| LOC14929 | 0.721 | 0.198 | 0.179 | 0.244 | 0.352 | 0.400 | 0.362 | 0.337 | 0.442 |
| SGK | 0.734 | 0.132 | 0.161 | 0.364 | 0.195 | 0.188 | 0.069 | −0.028 | −0.032 |
| HSPC152 | 0.735 | 0.135 | 0.252 | 0.138 | 0.032 | 0.015 | 0.067 | 0.305 | 0.316 |
| RPL30 | 0.736 | 0.448 | 0.468 | 0.448 | 0.318 | 0.110 | 0.264 | 0.059 | 0.120 |
| MASP1 | 0.737 | 0.108 | 0.040 | −0.005 | 0.083 | −0.150 | −0.041 | −0.201 | −0.066 |
| RPL36AL | 0.738 | 0.279 | 0.361 | 0.290 | 0.294 | 0.394 | 0.555 | 0.364 | 0.239 |
| Hs.187199 | 0.738 | 0.543 | 0.182 | 0.314 | 0.287 | 0.484 | 0.364 | 0.349 | 0.216 |
| JUND | 0.754 | 0.392 | 0.352 | 0.444 | 0.400 | 0.066 | 0.130 | 0.114 | 0.009 |
| SNX10 | 0.766 | 0.224 | 0.261 | 0.358 | 0.383 | 0.476 | 0.328 | 0.164 | 0.040 |
| MRCL3 | 0.776 | 0.248 | 0.387 | 0.471 | 0.418 | 0.290 | 0.283 | 0.139 | 0.144 |
| EBAG9 | 0.788 | 0.375 | 0.240 | 0.282 | 0.304 | 0.447 | 0.340 | 0.058 | −0.006 |
| CBX3 | 0.790 | 0.072 | 0.004 | 0.333 | 0.423 | 0.662 | 0.468 | 0.252 | 0.273 |
| CORTBP2 | 0.791 | 0.449 | 0.319 | 0.309 | 0.207 | 0.312 | 0.329 | 0.419 | 0.159 |
| GCNT2 | 0.794 | 0.062 | 0.104 | 0.040 | −0.292 | 0.204 | 0.235 | 0.122 | −0.179 |
| TSGA14 | 0.808 | 0.485 | 0.469 | 0.438 | 0.447 | 0.392 | 0.278 | 0.160 | 0.120 |
| Hs.444174 | 0.814 | 0.573 | 0.355 | 0.350 | 0.153 | 0.274 | 0.356 | 0.203 | 0.345 |
| Hs.61648 | 0.819 | 0.500 | 0.332 | 0.488 | 0.487 | 0.323 | 0.344 | 0.174 | 0.195 |
| CA3 | 0.820 | −0.008 | 0.027 | 0.353 | 0.007 | −0.117 | 0.036 | 0.245 | −0.259 |
| LOC37485 | 0.973 | 0.092 | 0.559 | 0.293 | 0.402 | 0.250 | 0.500 | 0.273 | 0.033 |
| KLHL6 | −1.323 | −0.205 | −0.123 | 0.086 | 0.043 | 0.128 | 0.015 | 0.334 | 0.341 |
| DVL1 | −1.118 | −0.337 | −0.165 | −0.346 | −0.075 | 0.111 | −0.412 | −0.142 | −0.080 |
| FXYD2 | −1.032 | −0.560 | −0.346 | −0.162 | 0.128 | −0.139 | 0.109 | −0.292 | −0.223 |
| PHKB | −1.019 | −0.215 | −0.363 | −0.210 | −0.278 | −0.058 | 0.029 | −0.132 | −0.176 |
| LOC34417 | −1.019 | −0.463 | −0.265 | −0.336 | 0.034 | −0.163 | −0.352 | −0.199 | −0.101 |
| STXBP4 | −1.004 | −0.162 | −0.383 | −0.443 | 0.032 | −0.148 | −0.085 | 0.035 | −0.310 |
| ITGB1BP1 | −0.968 | 0.030 | −0.063 | −0.058 | 0.076 | 0.241 | 0.056 | 0.149 | 0.294 |
| PSMB1 | −0.955 | −0.239 | −0.216 | −0.265 | −0.147 | −0.023 | −0.009 | −0.144 | −0.110 |
| LOC374744 | 0.938 | −0.187 | −0.270 | −0.282 | 0.018 | 0.041 | −0.112 | −0.060 | 0.054 |
| CYFIP2 | −0.935 | −0.459 | −0.199 | −0.266 | −0.357 | 0.020 | −0.022 | −0.214 | −0.276 |
| LOC40267 | −0.905 | −0.183 | 0.119 | −0.163 | −0.198 | −0.327 | −0.375 | −0.299 | −0.007 |
| MYH2 | −0.892 | −0.042 | −0.245 | 0.074 | −0.479 | −0.172 | 0.040 | −0.029 | −0.130 |
| NONO | −0.891 | −0.226 | −0.096 | −0.381 | −0.208 | 0.045 | −0.084 | −0.029 | −0.135 |
| LOC37614 | −0.888 | −0.302 | −0.131 | −0.347 | −0.265 | −0.142 | −0.014 | −0.128 | 0.000 |
| RAB6A | −0.880 | −0.428 | −0.140 | −0.279 | −0.049 | 0.076 | 0.029 | −0.040 | −0.080 |
| IHPK2 | −0.875 | −0.401 | −0.238 | −0.342 | −0.332 | 0.114 | −0.058 | −0.150 | −0.062 |
| FEN1 | −0.874 | −0.217 | −0.379 | −0.225 | −0.291 | 0.046 | −0.021 | −0.211 | −0.228 |
| RAD51L1 | −0.851 | −0.303 | −0.280 | −0.524 | −0.287 | −0.077 | −0.201 | −0.129 | −0.083 |
| LOC37613 | −0.846 | −0.259 | −0.207 | −0.355 | −0.074 | 0.083 | 0.244 | 0.090 | 0.171 |
| LOC14409 | −0.841 | −0.335 | −0.195 | −0.523 | −0.131 | −0.262 | −0.209 | −0.119 | −0.134 |
| GTF2B | −0.837 | −0.224 | −0.238 | −0.045 | −0.337 | −0.024 | −0.052 | −0.215 | −0.109 |
| GLG1 | −0.836 | −0.162 | −0.088 | −0.269 | −0.097 | 0.022 | −0.168 | −0.117 | −0.203 |
| MADH2 | −0.835 | −0.336 | −0.056 | −0.121 | −0.139 | 0.030 | 0.053 | −0.017 | 0.113 |
| LOC149934 | 0.807 | −0.369 | −0.362 | −0.273 | −0.102 | −0.295 | −0.148 | −0.021 | 0.118 |
| LOC37634 | −0.784 | −0.484 | −0.216 | −0.164 | −0.059 | 0.096 | 0.073 | −0.016 | −0.050 |
| LOC40217 | −0.779 | −0.114 | 0.043 | −0.168 | −0.116 | −0.280 | −0.024 | −0.258 | −0.199 |
| NFKBIB | −0.777 | −0.263 | −0.120 | −0.158 | −0.073 | −0.023 | −0.107 | 0.131 | 0.221 |
| MGC11257 | −0.775 | −0.360 | −0.278 | −0.291 | −0.241 | −0.352 | −0.317 | 0.215 | 0.046 |
| FLJ34969 | −0.774 | −0.392 | −0.096 | −0.163 | −0.127 | 0.369 | 0.013 | −0.075 | −0.203 |
| SLC5A2 | −0.766 | −0.371 | −0.076 | −0.260 | −0.224 | −0.314 | −0.147 | −0.065 | −0.132 |
| LOC14342 | −0.756 | 0.090 | 0.007 | −0.187 | −0.078 | −0.119 | 0.003 | −0.214 | −0.076 |
| FLJ21657 | −0.755 | −0.264 | −0.269 | −0.510 | 0.155 | −0.019 | −0.077 | 0.159 | 0.150 |
| SPTLC1 | −0.753 | −0.305 | −0.127 | −0.260 | −0.132 | 0.062 | 0.132 | 0.069 | −0.048 |
| ZNF227 | −0.748 | −0.214 | −0.118 | −0.050 | 0.084 | 0.171 | −0.032 | −0.147 | −0.004 |
| IGSF2 | −0.746 | −0.208 | −0.244 | −0.210 | −0.074 | −0.213 | −0.185 | −0.151 | −0.387 |
| CAP350 | −0.745 | −0.265 | −0.043 | −0.070 | −0.089 | 0.103 | 0.004 | −0.020 | −0.013 |
| TIM50L | −0.741 | −0.154 | −0.165 | −0.467 | −0.358 | −0.055 | −0.219 | −0.102 | −0.089 |
| LOC39251 | −0.733 | 0.068 | 0.200 | −0.026 | 0.058 | −0.038 | 0.029 | 0.020 | 0.121 |
| KIAA0053 | −0.725 | −0.181 | −0.408 | 0.079 | 0.086 | −0.367 | −0.049 | −0.023 | −0.418 |
| MGC2714 | −0.722 | −0.263 | −0.166 | −0.168 | −0.265 | 0.127 | 0.068 | 0.203 | 0.140 |
| DUSP6 | −0.721 | −0.116 | −0.429 | −0.131 | −0.155 | −0.130 | −0.273 | −0.215 | 0.007 |
| FLJ33810 | −0.717 | −0.109 | −0.306 | −0.397 | −0.166 | −0.135 | −0.091 | −0.274 | −0.109 |
| MRPL40 | −0.716 | −0.352 | −0.207 | −0.310 | −0.346 | −0.146 | −0.012 | −0.147 | −0.215 |
| SNRPN | −0.707 | −0.008 | 0.170 | −0.055 | −0.145 | −0.128 | −0.100 | 0.088 | 0.268 |
| ORC6L | −0.707 | −0.412 | −0.285 | −0.277 | −0.197 | −0.043 | −0.162 | −0.047 | 0.006 |
| XPC | −0.702 | −0.100 | −0.242 | −0.059 | 0.003 | −0.023 | 0.000 | 0.156 | 0.170 |
| ZNF226 | −0.690 | −0.205 | 0.014 | 0.051 | 0.201 | 0.188 | 0.253 | −0.020 | −0.100 |
| FLJ12875 | −0.686 | −0.026 | −0.241 | −0.252 | −0.227 | −0.007 | −0.050 | −0.079 | −0.017 |
| DDR1 | −0.684 | −0.395 | −0.052 | −0.266 | −0.206 | 0.067 | −0.025 | −0.433 | −0.133 |
| AE2 | −0.684 | −0.294 | −0.429 | −0.140 | −0.142 | 0.061 | 0.086 | −0.003 | 0.119 |
| HNRPD | −0.683 | −0.274 | −0.148 | −0.092 | 0.047 | 0.272 | 0.104 | 0.088 | 0.082 |
| LOC37521 | −0.679 | −0.274 | −0.223 | −0.201 | 0.002 | 0.003 | 0.042 | −0.030 | 0.091 |
| L3MBTL2 | −0.677 | −0.360 | −0.221 | −0.085 | −0.059 | 0.114 | −0.050 | 0.215 | 0.176 |
| RAET1E | −0.676 | −0.273 | 0.027 | −0.227 | −0.050 | −0.246 | 0.088 | −0.044 | 0.185 |
| HIST1H3H | −0.675 | −0.244 | −0.385 | −0.131 | −0.131 | −0.095 | −0.077 | −0.294 | −0.302 |
| UPK3B | −0.668 | −0.530 | −0.191 | −0.346 | −0.076 | −0.271 | −0.140 | −0.122 | 0.072 |
| FLJ23235 | −0.663 | −0.337 | −0.169 | −0.478 | −0.005 | −0.051 | −0.029 | 0.067 | 0.021 |
| PDGFC | −0.660 | −0.405 | −0.044 | 0.119 | 0.155 | 0.014 | 0.068 | −0.133 | −0.073 |
| TRA2A | −0.656 | −0.268 | −0.133 | −0.247 | 0.150 | 0.218 | 0.166 | −0.070 | −0.078 |
| NY-SAR- | 0.647 | −0.489 | −0.085 | −0.337 | −0.020 | −0.093 | 0.094 | −0.069 | −0.014 |
| 41 | | | | | | | | | |
| RNF39 | −0.647 | −0.125 | −0.336 | −0.044 | −0.053 | −0.166 | −0.125 | 0.059 | −0.078 |
| C9orf39 | −0.646 | 0.137 | 0.039 | −0.235 | −0.298 | 0.222 | −0.183 | −0.118 | −0.017 |
| COPE | −0.637 | −0.374 | −0.046 | −0.302 | −0.195 | 0.067 | 0.078 | −0.132 | −0.093 |
| GABRB3 | −0.637 | −0.292 | −0.009 | 0.127 | −0.031 | −0.043 | 0.044 | −0.206 | 0.001 |
| LOC40032 | −0.635 | −0.260 | −0.242 | −0.259 | −0.252 | −0.079 | −0.162 | 0.068 | 0.133 |
| TUWD12 | −0.635 | −0.104 | −0.047 | −0.099 | 0.113 | 0.385 | 0.205 | −0.040 | 0.053 |
| NNMT | −0.631 | 0.135 | 0.131 | 0.186 | −0.350 | −0.109 | 0.456 | −0.065 | 0.104 |
| LOC40058 | −0.629 | −0.098 | −0.027 | −0.019 | 0.069 | −0.185 | −0.086 | −0.319 | −0.125 |
| PDE1B | −0.627 | −0.054 | −0.275 | −0.285 | −0.232 | −0.108 | −0.139 | −0.255 | −0.133 |
| SLC7A1 | −0.626 | −0.274 | −0.119 | −0.189 | −0.059 | −0.145 | −0.244 | −0.334 | 0.028 |
| LOC15487 | −0.626 | 0.130 | −0.020 | 0.035 | −0.072 | −0.416 | −0.138 | 0.109 | −0.115 |
| LOC37578 | −0.625 | −0.145 | −0.296 | −0.327 | −0.196 | −0.053 | −0.258 | −0.079 | 0.061 |
| C6orf170 | −0.623 | −0.245 | −0.023 | −0.217 | −0.011 | −0.187 | −0.026 | −0.050 | −0.064 |
| KIAA0073 | −0.623 | −0.148 | −0.152 | −0.108 | −0.090 | −0.115 | −0.193 | −0.061 | −0.054 |
| MRPL45 | −0.622 | −0.350 | −0.281 | −0.358 | −0.267 | −0.125 | 0.005 | 0.084 | 0.090 |
| HAMP | −0.622 | −0.161 | −0.164 | −0.272 | −0.197 | −0.302 | 0.115 | −0.100 | −0.137 |
| THBS4 | −0.619 | 0.102 | 0.218 | −0.085 | −0.774 | 0.037 | 0.101 | 0.123 | 0.165 |
| HRPT2 | −0.616 | −0.455 | −0.199 | −0.132 | −0.232 | 0.136 | 0.014 | 0.091 | −0.035 |
| KIF11 | −0.611 | −0.002 | −0.111 | −0.141 | −0.061 | 0.076 | 0.057 | −0.422 | −0.485 |
| PEX3 | −0.611 | −0.558 | −0.003 | 0.152 | 0.043 | 0.214 | 0.297 | −0.049 | −0.124 |
| CASC1 | −0.611 | −0.091 | 0.051 | 0.099 | −0.011 | −0.198 | −0.068 | −0.112 | 0.062 |
| CORT | −0.610 | −0.236 | −0.173 | −0.181 | −0.228 | −0.214 | 0.007 | −0.128 | −0.095 |
| NUBP2 | −0.608 | −0.360 | −0.341 | −0.235 | −0.049 | −0.153 | −0.089 | −0.038 | −0.090 |
| LOC39987 | −0.608 | −0.333 | −0.178 | −0.069 | −0.276 | −0.050 | −0.100 | −0.081 | −0.120 |
| CDC2L1 | −0.603 | −0.197 | 0.040 | −0.193 | −0.074 | −0.174 | −0.168 | −0.267 | −0.177 |
| LOC285194 | 0.602 | −0.184 | −0.255 | −0.093 | −0.026 | 0.011 | −0.476 | −0.377 | −0.196 |
| RPL13A | −0.601 | −0.419 | −0.264 | −0.312 | −0.058 | 0.100 | 0.048 | −0.312 | −0.286 |
| MGC48625 | −0.597 | 0.025 | 0.067 | 0.083 | 0.248 | 0.004 | −0.257 | −0.327 | −0.051 |
| GABRQ | −0.597 | −0.251 | 0.124 | −0.164 | −0.349 | −0.054 | −0.206 | −0.497 | −0.384 |
| LOC374954 | 0.597 | −0.180 | −0.122 | −0.137 | 0.000 | −0.137 | −0.006 | −0.109 | −0.055 |
| SNRPD2 | −0.596 | −0.301 | −0.276 | −0.117 | 0.069 | −0.139 | 0.109 | 0.179 | 0.086 |
| C12orf5 | −0.596 | −0.368 | −0.214 | −0.107 | −0.081 | −0.085 | 0.090 | 0.042 | −0.002 |
| FASTK | −0.596 | −0.134 | −0.118 | −0.172 | −0.175 | −0.012 | −0.087 | −0.423 | −0.046 |
| LOC12849 | −0.595 | −0.406 | 0.050 | −0.139 | 0.073 | −0.066 | −0.007 | 0.161 | −0.013 |
| DNA2L | −0.594 | −0.161 | −0.238 | −0.122 | −0.266 | 0.018 | −0.072 | −0.256 | −0.402 |
| RASGRP1 | −0.593 | −0.465 | −0.393 | −0.280 | 0.095 | 0.092 | 0.034 | −0.411 | −0.361 |
| KTN1 | −0.591 | −0.298 | −0.141 | −0.191 | 0.008 | 0.321 | 0.199 | 0.016 | 0.031 |
| C6orf46 | −0.590 | −0.156 | −0.101 | −0.065 | −0.170 | −0.226 | 0.105 | 0.080 | −0.116 |
| LOC388274 | 0.590 | −0.163 | 0.063 | 0.326 | −0.003 | 0.311 | −0.451 | 0.133 | 0.028 |
| SIRT2 | −0.589 | −0.281 | −0.241 | −0.145 | −0.348 | 0.118 | −0.192 | −0.212 | −0.097 |
| FLJ20105 | −0.588 | −0.375 | −0.090 | −0.229 | −0.171 | −0.009 | 0.007 | −0.131 | −0.184 |
| FLJ13052 | −0.587 | −0.193 | −0.247 | −0.042 | 0.057 | 0.073 | 0.008 | 0.359 | 0.331 |
| RPL7A | −0.587 | −0.117 | −0.125 | −0.131 | −0.146 | −0.160 | −0.169 | −0.281 | −0.310 |
| LOC389974 | 0.585 | −0.134 | −0.159 | −0.092 | 0.199 | 0.117 | 0.096 | −0.056 | 0.102 |

TABLE 4
Genes screened for pattern set 3 (S3-T3-Pattern-III).

| Gene | 2 nm | 5 nm | 10 nm | 15 nm | 20 nm | 30 nm | 40 nm | 80 nm | 200 nm |
|---|---|---|---|---|---|---|---|---|---|
| H2AV | −0.666 | −0.334 | −0.081 | −0.334 | −0.235 | −1.018 | −0.819 | −0.470 | −0.667 |
| LOC12943 | −0.824 | −0.329 | −0.062 | −0.742 | −0.901 | −0.924 | −0.746 | −0.547 | −0.170 |
| ALDOA | −0.302 | 0.045 | 0.337 | −0.327 | −0.651 | −0.791 | −0.587 | −0.380 | −0.101 |
| Hs.474368 | −0.393 | 0.174 | 0.018 | −0.377 | −0.621 | −0.777 | −0.653 | −0.553 | −0.583 |
| FKBP1A | −0.226 | −0.178 | −0.103 | −0.256 | −0.485 | −0.764 | −0.663 | −0.635 | −0.512 |
| PSMD4 | −0.198 | −0.128 | −0.113 | −0.209 | −0.492 | −0.739 | −0.408 | −0.323 | −0.121 |
| ACINUS | 0.015 | −0.104 | −0.145 | −0.268 | −0.404 | −0.713 | −0.519 | −0.423 | −0.419 |
| LOC25382 | −0.721 | −0.613 | −0.453 | −0.768 | −0.521 | −0.697 | −0.622 | −0.428 | −0.464 |
| ILF3 | −0.264 | −0.022 | 0.083 | −0.202 | −0.478 | −0.694 | −0.385 | −0.498 | −0.466 |
| CDC25B | −0.420 | −0.346 | −0.168 | −0.547 | −0.414 | −0.683 | −0.518 | −0.513 | −0.565 |
| DKFZp434 | −0.344 | −0.227 | −0.339 | −0.422 | −0.516 | −0.677 | −0.400 | −0.573 | −0.115 |
| PSCD2 | −0.257 | −0.342 | −0.422 | −0.295 | −0.401 | −0.670 | −0.541 | −0.497 | −0.390 |
| SUMF2 | −0.327 | −0.437 | −0.160 | −0.340 | −0.472 | −0.668 | −0.560 | −0.569 | −0.704 |
| NUTF2 | −0.228 | −0.116 | −0.105 | −0.397 | −0.401 | −0.657 | −0.383 | −0.387 | −0.229 |
| EIF5A | −0.270 | 0.123 | −0.052 | −0.286 | −0.369 | −0.657 | −0.623 | −0.075 | 0.153 |
| PSMC4 | −0.440 | −0.079 | 0.023 | −0.340 | −0.568 | −0.657 | −0.558 | −0.131 | −0.031 |
| RNPS1 | −0.451 | −0.174 | −0.045 | −0.537 | −0.528 | −0.655 | −0.605 | −0.351 | −0.339 |
| CALM3 | −0.183 | −0.194 | −0.206 | −0.567 | −0.442 | −0.649 | −0.492 | −0.380 | −0.391 |
| CG012 | −0.203 | 0.011 | −0.406 | −0.343 | −0.404 | −0.641 | −0.389 | −0.235 | −0.132 |
| TAF6L | −0.138 | −0.051 | −0.118 | −0.311 | −0.295 | −0.622 | −0.547 | −0.414 | −0.360 |
| HIP-55 | −0.175 | 0.063 | −0.095 | −0.101 | −0.070 | −0.610 | −0.384 | −0.275 | −0.321 |
| MGC4809 | −0.480 | 0.020 | −0.124 | −0.118 | −0.474 | −0.606 | −0.467 | −0.735 | −0.493 |
| FLJ20512 | −0.037 | 0.021 | 0.215 | −0.161 | −0.205 | −0.606 | −0.418 | −0.393 | −0.331 |
| APEX1 | −0.377 | −0.223 | −0.250 | −0.331 | −0.517 | −0.605 | −0.417 | −0.062 | 0.187 |
| MTND1 | −0.549 | −0.085 | −0.268 | −0.285 | −0.458 | −0.604 | −0.571 | −0.595 | −0.457 |
| RUVBL2 | −0.525 | −0.293 | −0.201 | −0.377 | −0.483 | −0.603 | −0.393 | −0.128 | −0.069 |
| CSK | −0.310 | −0.203 | −0.372 | −0.442 | −0.487 | −0.602 | −0.626 | −0.087 | −0.247 |
| C6orf29 | −0.757 | −0.436 | −0.307 | −0.698 | −0.385 | −0.590 | −0.422 | −0.311 | −0.385 |
| PSMB8 | −0.165 | −0.041 | −0.074 | −0.373 | −0.605 | −0.551 | −0.485 | −0.340 | −0.268 |
| TPI1 | −0.380 | −0.267 | −0.331 | −0.410 | −0.587 | −0.538 | −0.521 | 0.131 | 0.195 |
| TUBA6 | −0.623 | −0.466 | −0.400 | −0.594 | −0.692 | −0.538 | −0.453 | −0.505 | −0.366 |
| ZNRD1 | −0.305 | −0.391 | −0.328 | −0.441 | −0.612 | −0.501 | −0.354 | −0.318 | −0.250 |
| LOC13447 | −0.656 | −0.325 | −0.132 | −0.608 | −0.779 | −0.404 | −0.424 | −0.198 | −0.107 |
| C9orf97 | −0.271 | −0.136 | −0.146 | −0.502 | −0.304 | −0.281 | −0.700 | −0.233 | −0.124 |
| PTCRA | −0.189 | −0.092 | 0.063 | −0.360 | −0.800 | −0.168 | −0.402 | −0.194 | −0.381 |
| RNF17 | 0.335 | 0.263 | 0.030 | 0.003 | 0.345 | 0.084 | 0.588 | 0.215 | 0.231 |
| LOC37508 | 0.394 | 0.050 | 0.097 | 0.071 | 0.240 | 0.187 | 0.621 | 0.369 | 0.371 |
| RPL26 | 0.324 | 0.093 | 0.187 | 0.445 | 0.473 | 0.517 | 0.605 | 0.342 | 0.269 |
| LOC37765 | −0.211 | −0.173 | 0.136 | 0.187 | 0.669 | 0.532 | 0.423 | 0.335 | 0.360 |
| LOC90701 | −0.379 | −0.219 | −0.025 | 0.107 | 0.419 | 0.553 | 0.588 | 0.238 | 0.205 |
| C10orf88 | −0.124 | 0.104 | 0.184 | 0.264 | 0.416 | 0.558 | 0.600 | 0.474 | 0.425 |
| GMFB | −0.275 | −0.319 | −0.013 | −0.019 | 0.266 | 0.571 | 0.614 | 0.288 | 0.360 |
| NR2F2 | 0.145 | 0.253 | 0.178 | 0.388 | 0.367 | 0.586 | 0.288 | 0.202 | −0.031 |
| LOC37481 | −0.556 | −0.320 | −0.386 | −0.161 | 0.313 | 0.586 | 0.304 | 0.170 | 0.349 |
| RAP2C | 0.578 | 0.089 | −0.010 | 0.246 | 0.444 | 0.589 | 0.508 | 0.158 | 0.159 |
| ZNF146 | −0.429 | −0.089 | 0.055 | −0.206 | 0.243 | 0.590 | 0.308 | 0.446 | 0.332 |
| STAG2 | −0.097 | −0.223 | 0.105 | 0.129 | 0.283 | 0.592 | 0.352 | 0.070 | 0.022 |
| TUBA3 | 0.135 | 0.250 | 0.043 | 0.291 | 0.459 | 0.592 | 0.420 | 0.245 | 0.278 |
| KCNK1 | 0.145 | −0.096 | 0.008 | −0.103 | 0.237 | 0.595 | 0.407 | 0.273 | 0.404 |
| SRP9 | 0.113 | 0.247 | 0.238 | 0.425 | 0.164 | 0.597 | 0.398 | 0.029 | −0.003 |
| Hs.58104 | 0.464 | 0.285 | −0.136 | 0.273 | 0.226 | 0.600 | 0.699 | 0.494 | 0.516 |
| USP1 | −0.285 | −0.422 | −0.333 | −0.174 | 0.033 | 0.600 | 0.401 | 0.053 | 0.010 |
| ST13 | −0.370 | −0.272 | −0.283 | −0.021 | 0.228 | 0.600 | 0.414 | 0.753 | 0.658 |
| LOC51668 | 0.087 | −0.035 | 0.012 | 0.160 | 0.419 | 0.602 | 0.540 | 0.290 | 0.423 |
| HIP14 | −0.041 | 0.031 | −0.043 | 0.193 | 0.299 | 0.604 | 0.439 | 0.348 | 0.257 |
| MGC8721 | 0.039 | 0.063 | 0.132 | 0.217 | 0.381 | 0.605 | 0.496 | 0.295 | 0.291 |
| C14orf104 | 0.134 | 0.109 | 0.011 | 0.093 | 0.165 | 0.606 | 0.346 | 0.662 | 0.764 |
| FKSG14 | 0.217 | 0.050 | 0.251 | 0.235 | 0.345 | 0.608 | 0.523 | 0.122 | 0.186 |
| MAD2L1 | −0.344 | −0.376 | −0.096 | 0.162 | 0.420 | 0.610 | 0.558 | 0.245 | 0.190 |
| CDC7 | −0.211 | −0.243 | −0.095 | −0.119 | 0.064 | 0.612 | 0.438 | 0.062 | −0.147 |
| SYBL1 | 0.081 | 0.146 | 0.124 | 0.298 | 0.199 | 0.614 | 0.396 | 0.133 | 0.093 |
| FLJ32803 | 0.400 | 0.239 | 0.019 | 0.302 | 0.449 | 0.615 | 0.468 | 0.584 | 0.393 |
| SIRT1 | 0.026 | 0.084 | 0.113 | 0.196 | 0.232 | 0.619 | 0.406 | 0.183 | 0.232 |
| VBP1 | 0.173 | 0.075 | 0.069 | 0.203 | 0.317 | 0.619 | 0.363 | 0.162 | 0.245 |
| PSMC2 | 0.367 | −0.245 | −0.063 | 0.153 | 0.315 | 0.620 | 0.381 | 0.451 | 0.386 |
| HAT1 | −0.447 | −0.337 | −0.250 | −0.054 | 0.165 | 0.621 | 0.450 | 0.096 | 0.106 |
| NXT2 | −0.535 | −0.256 | −0.508 | −0.104 | 0.012 | 0.621 | 0.432 | 0.407 | 0.116 |
| hIAN2 | 0.081 | −0.242 | −0.041 | 0.074 | 0.564 | 0.622 | 0.405 | 0.646 | 0.419 |
| C10orf22 | 0.307 | 0.213 | 0.235 | 0.365 | 0.474 | 0.623 | 0.384 | 0.433 | 0.248 |
| DKFZp547 | 0.326 | 0.177 | 0.117 | 0.293 | 0.305 | 0.626 | 0.450 | 0.324 | 0.326 |
| LIPA | 0.601 | 0.186 | 0.234 | 0.330 | 0.399 | 0.629 | 0.564 | 0.341 | 0.304 |
| VPS4B | 0.218 | −0.006 | −0.044 | 0.088 | 0.185 | 0.633 | 0.399 | 0.275 | 0.305 |
| SACM1L | −0.084 | −0.259 | 0.057 | −0.045 | 0.289 | 0.634 | 0.250 | 0.535 | 0.536 |
| RAD21 | 0.613 | −0.075 | 0.047 | 0.135 | 0.160 | 0.634 | 0.403 | −0.137 | −0.001 |
| MGC44593 | −0.281 | −0.046 | 0.199 | 0.293 | 0.236 | 0.638 | 0.623 | −0.424 | −0.322 |
| RPGR | 0.756 | 0.085 | 0.205 | 0.357 | 0.433 | 0.644 | 0.623 | 0.338 | 0.373 |
| LOC37672 | 0.411 | 0.369 | 0.148 | 0.552 | 0.440 | 0.644 | 0.412 | 0.342 | 0.271 |
| GNG10 | −0.130 | −0.092 | −0.148 | 0.017 | 0.245 | 0.644 | 0.460 | 0.160 | 0.146 |
| ITM2B | 0.573 | −0.136 | 0.139 | 0.233 | 0.325 | 0.644 | 0.308 | 0.105 | 0.095 |
| SMN1 | −0.203 | −0.483 | −0.144 | 0.082 | 0.421 | 0.645 | 0.409 | 0.564 | 0.369 |
| DYRK1A | 0.083 | −0.183 | −0.179 | 0.047 | 0.283 | 0.645 | 0.450 | 0.028 | 0.059 |
| NUSAP1 | −0.243 | −0.196 | −0.089 | 0.070 | 0.185 | 0.645 | 0.324 | −0.052 | −0.140 |
| SAP30 | 0.250 | −0.132 | 0.029 | 0.085 | 0.415 | 0.646 | 0.362 | 0.391 | 0.313 |
| AUP1 | −0.182 | −0.195 | −0.035 | −0.086 | 0.088 | 0.655 | 0.315 | 0.371 | 0.416 |
| TDE2 | −0.016 | −0.091 | 0.183 | 0.248 | 0.179 | 0.663 | 0.536 | 0.172 | 0.252 |
| DNCL1 | 0.201 | 0.205 | 0.249 | 0.347 | 0.480 | 0.663 | 0.663 | 0.333 | 0.367 |
| TNFAIP8 | 0.432 | 0.294 | 0.414 | 0.512 | 0.558 | 0.665 | 0.586 | 0.085 | 0.066 |
| SNX16 | 0.395 | 0.271 | 0.391 | 0.346 | 0.371 | 0.667 | 0.485 | 0.437 | 0.400 |
| MINPP1 | 0.077 | −0.344 | −0.159 | 0.069 | 0.100 | 0.667 | 0.148 | 0.452 | 0.320 |
| LSM5 | 0.277 | 0.035 | 0.224 | 0.334 | 0.581 | 0.671 | 0.617 | 0.471 | 0.465 |
| PAQR3 | 0.385 | 0.050 | 0.099 | 0.266 | 0.356 | 0.675 | 0.576 | 0.123 | 0.187 |
| RBBP7 | 0.068 | 0.001 | −0.092 | 0.272 | 0.305 | 0.686 | 0.575 | 0.359 | 0.262 |
| C14orf142 | 0.180 | 0.066 | 0.193 | 0.346 | 0.390 | 0.687 | 0.456 | 0.247 | 0.290 |
| HIF1A | −0.303 | 0.075 | −0.032 | 0.099 | 0.212 | 0.688 | 0.284 | 0.061 | 0.238 |
| LOC92912 | 0.056 | −0.151 | −0.157 | 0.085 | 0.456 | 0.690 | 0.468 | 0.320 | 0.371 |
| CLIC4 | 0.289 | 0.255 | 0.206 | 0.325 | 0.480 | 0.691 | 0.542 | 0.130 | 0.071 |
| GPR160 | 0.578 | 0.328 | 0.474 | 0.530 | 0.581 | 0.694 | 0.648 | 0.176 | 0.377 |
| LOC15499 | 0.171 | 0.061 | 0.222 | 0.401 | 0.614 | 0.696 | 0.672 | 0.408 | 0.424 |
| LOC37729 | −0.717 | −0.526 | −0.441 | −0.121 | 0.591 | 0.697 | 0.570 | 0.471 | 0.469 |
| ACSL3 | 0.239 | 0.426 | 0.095 | 0.011 | 0.471 | 0.698 | 0.374 | 0.389 | 0.237 |
| TAF9L | 0.251 | −0.009 | 0.003 | 0.135 | 0.139 | 0.699 | 0.436 | 0.042 | −0.059 |
| LOC20091 | 0.725 | 0.207 | 0.152 | 0.257 | 0.580 | 0.700 | 0.684 | 0.460 | 0.491 |
| HNRPH3 | 0.138 | 0.084 | 0.005 | 0.324 | 0.221 | 0.701 | 0.417 | 0.178 | 0.182 |
| LOC40121 | −0.227 | −0.302 | −0.198 | −0.212 | 0.304 | 0.724 | 0.404 | 0.665 | 0.578 |
| LOC40000 | −0.340 | −0.584 | −0.446 | −0.124 | 0.373 | 0.724 | 0.374 | 0.522 | 0.485 |
| ATP5L | 0.095 | 0.186 | 0.237 | 0.362 | 0.527 | 0.727 | 0.661 | 0.366 | 0.299 |
| SNRPB2 | 0.236 | 0.017 | 0.078 | 0.139 | 0.335 | 0.730 | 0.594 | 0.471 | 0.476 |
| JJAZ1 | 0.180 | 0.054 | 0.211 | 0.270 | 0.444 | 0.730 | 0.633 | 0.380 | 0.332 |
| HNRPK | −0.251 | −0.352 | −0.240 | −0.031 | 0.406 | 0.732 | 0.378 | 0.441 | 0.363 |
| Hs.28465 | −0.046 | 0.063 | −0.625 | 0.128 | 0.015 | 0.739 | 0.473 | 0.103 | 0.102 |
| SMARCA5 | −0.188 | −0.059 | −0.246 | 0.206 | 0.452 | 0.740 | 0.484 | 0.442 | 0.338 |
| OSBPL8 | −0.167 | −0.213 | −0.154 | 0.097 | 0.344 | 0.740 | 0.417 | 0.135 | 0.099 |
| HSPC039 | −0.328 | −0.275 | −0.031 | 0.072 | 0.148 | 0.745 | 0.623 | 0.341 | 0.129 |
| BCAS2 | −0.010 | −0.137 | 0.185 | 0.072 | 0.684 | 0.749 | 0.684 | 0.527 | 0.470 |
| RPL7 | −0.498 | −0.612 | −0.375 | −0.221 | 0.120 | 0.752 | 0.487 | 0.280 | 0.276 |
| FLJ20647 | −0.174 | 0.118 | 0.100 | 0.094 | 0.179 | 0.758 | 0.559 | 0.002 | −0.071 |
| SP3 | 0.398 | 0.130 | 0.142 | 0.224 | 0.330 | 0.762 | 0.414 | 0.182 | 0.212 |
| LOC28555 | 0.203 | 0.107 | 0.219 | −0.125 | 0.489 | 0.766 | 0.628 | 0.235 | 0.335 |
| UBQLN2 | −0.038 | 0.118 | 0.128 | 0.249 | 0.166 | 0.771 | 0.211 | 0.655 | 0.553 |
| SSBP1 | 0.088 | −0.016 | 0.013 | 0.287 | 0.499 | 0.774 | 0.512 | 0.577 | 0.418 |
| SFPQ | −0.401 | −0.336 | −0.071 | 0.211 | 0.442 | 0.785 | 0.462 | 0.191 | 0.168 |
| PGRMC1 | 0.425 | 0.113 | 0.018 | 0.159 | 0.291 | 0.786 | 0.561 | 0.353 | 0.354 |
| TMSL1 | −0.612 | −0.718 | −0.519 | −0.380 | 0.297 | 0.801 | 0.459 | 0.711 | 0.753 |
| LZTFL1 | 0.626 | 0.120 | 0.184 | 0.497 | 0.484 | 0.814 | 0.625 | 0.597 | 0.488 |
| GAS41 | 0.166 | 0.104 | 0.075 | 0.352 | 0.359 | 0.814 | 0.559 | −0.049 | −0.107 |
| KHDRBS1 | 0.558 | 0.215 | 0.267 | 0.424 | 0.445 | 0.827 | 0.636 | 0.138 | 0.053 |
| NOP5/NOP | 0.360 | −0.024 | 0.090 | 0.275 | 0.481 | 0.832 | 0.682 | 0.637 | 0.641 |
| KIN | 0.159 | −0.047 | 0.020 | 0.347 | 0.493 | 0.842 | 0.640 | 0.390 | 0.415 |
| NUCB2 | 0.046 | −0.183 | −0.133 | 0.203 | 0.550 | 0.958 | 0.597 | 0.241 | 0.174 |
| HIG1 | 0.023 | −0.184 | −0.105 | 0.209 | 0.564 | 1.050 | 0.705 | 0.594 | 0.494 |
| RPL9 | −0.494 | −0.728 | −0.491 | −0.295 | 0.380 | 1.111 | 0.918 | 0.572 | 0.637 |
| NAP1L1 | 0.292 | −0.024 | 0.015 | 0.268 | 0.227 | 0.608 | 0.353 | 0.210 | 0.147 |
| H2AV | 0.531 | 0.305 | 0.280 | 0.577 | 0.871 | −0.009 | −4.820 | −4.704 | 0.204 |
| LOC12943 | −0.329 | −0.182 | −0.984 | −1.893 | 0.375 | −1.843 | −0.256 | −0.005 | 0.361 |
| ALDOA | −0.203 | 0.184 | −0.667 | −1.216 | 0.213 | −1.824 | −0.268 | −0.155 | 0.078 |
| Hs.474368 | −0.344 | 0.070 | 0.164 | −0.362 | 0.178 | −0.802 | −0.418 | −0.043 | −0.094 |
| FKBP1A | −0.046 | −0.071 | 0.163 | −0.108 | −0.121 | −0.728 | −0.627 | −0.268 | −0.113 |
| PSMD4 | 0.065 | 0.129 | −0.404 | −0.417 | −0.171 | −0.965 | −0.427 | −0.356 | 0.040 |
| ACINUS | 0.038 | 0.020 | −0.044 | −0.428 | −0.099 | −0.626 | −0.533 | −0.311 | −0.184 |
| LOC25382 | −0.645 | −0.212 | −0.662 | −0.253 | −0.321 | −0.799 | −0.231 | −0.151 | −0.259 |
| ILF3 | −0.291 | −0.070 | −0.635 | −0.969 | −0.055 | −1.520 | −0.483 | −0.225 | 0.135 |
| CDC25B | −0.167 | −0.033 | 0.041 | −0.525 | 0.007 | −0.519 | −0.652 | −0.128 | −0.151 |
| DKFZp434 | −0.258 | −0.078 | −0.288 | −0.525 | −0.054 | −0.757 | −0.262 | −0.300 | −0.113 |
| PSCD2 | 0.077 | 0.072 | 0.206 | −0.035 | 0.017 | −0.608 | −0.183 | −0.092 | −0.187 |
| SUMF2 | −0.250 | −0.153 | −0.465 | −0.697 | −0.183 | −0.898 | −0.371 | −0.321 | −0.307 |
| NUTF2 | −0.241 | −0.180 | −0.184 | −0.709 | −0.128 | −0.970 | −0.407 | −0.275 | −0.228 |
| EIF5A | −0.168 | 0.063 | −0.713 | −1.196 | 0.048 | −1.302 | −0.962 | −0.385 | 0.202 |
| PSMC4 | −0.045 | 0.084 | −0.731 | −1.082 | 0.121 | −1.174 | −0.160 | −0.169 | 0.185 |
| RNPS1 | −0.116 | 0.014 | −0.279 | −0.785 | 0.007 | −0.737 | −0.745 | −0.075 | 0.052 |
| CALM3 | 0.058 | −0.084 | −0.319 | −0.454 | −0.089 | −0.454 | −0.632 | −0.274 | −0.141 |
| CG012 | −0.368 | −0.459 | −0.374 | −0.267 | −0.445 | −0.749 | −0.441 | −0.391 | −0.389 |
| TAF6L | 0.042 | 0.031 | 0.124 | −0.022 | −0.128 | −0.595 | −0.638 | −0.206 | −0.126 |
| HIP-55 | −0.140 | −0.083 | −0.117 | 0.010 | −0.379 | −0.902 | −0.266 | −0.246 | −0.254 |
| MGC4809 | −0.104 | −0.169 | 0.067 | 0.055 | −0.309 | −0.716 | −0.461 | −0.101 | −0.420 |
| FLJ20512 | 0.052 | 0.018 | −0.001 | −0.260 | −0.017 | −0.808 | −0.394 | −0.255 | −0.197 |
| APEX1 | −0.110 | −0.110 | −0.979 | −1.042 | 0.181 | −0.856 | −0.243 | −0.020 | 0.198 |
| MTND1 | −0.115 | 0.007 | −0.441 | −0.485 | −0.069 | −0.731 | −0.475 | −0.251 | 0.274 |
| RUVBL2 | −0.074 | −0.062 | 0.035 | −0.185 | 0.054 | −0.681 | −0.271 | −0.278 | −0.093 |
| CSK | 0.123 | −0.007 | −0.067 | −0.162 | −0.202 | −0.283 | −0.798 | −0.286 | −0.345 |
| C6orf29 | −0.847 | −0.379 | −0.936 | −0.514 | −0.280 | −0.900 | −0.188 | −0.144 | −0.339 |
| PSMB8 | −0.036 | 0.190 | −0.182 | −0.574 | 0.158 | −0.703 | −0.106 | −0.035 | 0.070 |
| TPI1 | −0.008 | 0.056 | −0.176 | −0.516 | −0.039 | −0.739 | −0.313 | −0.155 | −0.050 |
| TUBA6 | −0.212 | 0.062 | −0.008 | −0.677 | 0.110 | −1.052 | −0.143 | −0.008 | −0.055 |
| ZNRD1 | −0.188 | −0.116 | −0.307 | −0.357 | −0.034 | −0.654 | −0.527 | −0.160 | −0.215 |
| LOC13447 | −0.044 | 0.130 | −0.055 | −0.832 | 0.278 | −0.718 | 0.029 | 0.012 | 0.081 |
| C9orf97 | −0.503 | −0.427 | −0.660 | −0.193 | −0.229 | −0.676 | −0.165 | −0.208 | 0.075 |
| PTCRA | −0.591 | −0.271 | −0.437 | −0.105 | −0.330 | −0.662 | −0.382 | −0.295 | −0.025 |
| RNF17 | 0.289 | −0.052 | 0.330 | 0.222 | 0.244 | 0.616 | 0.215 | 0.139 | 0.237 |
| LOC37508 | 0.094 | 0.083 | 0.193 | 0.351 | 0.281 | 0.630 | 0.512 | 0.296 | 0.152 |
| RPL26 | 0.259 | 0.112 | 0.662 | 0.973 | 0.238 | 0.911 | 0.119 | 0.471 | 0.337 |
| LOC37765 | 0.304 | 0.132 | 0.280 | 0.376 | −0.034 | 0.798 | 0.693 | 0.311 | −0.187 |
| LOC90701 | 0.329 | 0.159 | 0.024 | 0.189 | 0.113 | 0.774 | 0.679 | 0.422 | 0.109 |
| C10orf88 | −0.083 | −0.225 | −0.165 | 0.023 | 0.093 | 0.670 | 0.332 | 0.054 | −0.117 |
| GMFB | 0.007 | 0.003 | 0.164 | 0.224 | 0.081 | 0.789 | 0.199 | 0.147 | 0.093 |
| NR2F2 | 0.363 | 0.338 | 0.141 | 0.192 | 0.359 | 0.752 | 0.497 | 0.344 | 0.192 |
| LOC37481 | 0.100 | 0.208 | −0.107 | 0.134 | 0.269 | 0.723 | 0.598 | 0.406 | 0.012 |
| RAP2C | −0.366 | −0.178 | −0.284 | 0.047 | 0.062 | 0.833 | 0.284 | 0.190 | 0.090 |
| ZNF146 | 0.020 | 0.245 | 0.069 | 0.180 | 0.133 | 0.734 | 0.708 | 0.189 | 0.120 |
| STAG2 | −0.472 | −0.126 | −0.032 | −0.173 | 0.146 | 0.659 | 0.443 | 0.159 | 0.077 |
| TUBA3 | 0.279 | 0.053 | −0.019 | 0.210 | 0.267 | 0.677 | 0.514 | 0.340 | 0.272 |
| KCNK1 | −0.113 | −0.104 | −0.084 | −0.086 | −0.189 | 0.322 | 0.755 | 0.210 | −0.024 |
| SRP9 | 0.079 | 0.270 | 0.236 | 0.360 | 0.211 | 0.680 | 0.532 | 0.240 | 0.270 |
| Hs.58104 | 0.340 | 0.205 | 0.453 | 0.610 | 0.134 | 0.715 | 0.722 | 0.266 | 0.066 |
| USP1 | −0.237 | −0.212 | −0.042 | 0.185 | 0.416 | 0.788 | 0.744 | 0.415 | 0.315 |
| ST13 | −0.050 | −0.041 | 0.149 | −0.156 | 0.109 | 0.586 | 0.292 | 0.329 | 0.192 |
| LOC51668 | −0.029 | 0.203 | −0.082 | 0.263 | 0.138 | 0.592 | 0.695 | 0.355 | 0.015 |
| HIP14 | 0.399 | 0.125 | 0.132 | 0.340 | 0.438 | 0.793 | 0.481 | 0.347 | 0.349 |
| MGC8721 | 0.138 | −0.057 | 0.161 | 0.153 | 0.236 | 0.687 | 0.633 | 0.271 | 0.382 |
| C14orf104 | −0.053 | 0.224 | 0.255 | 0.313 | 0.288 | 0.988 | 0.520 | 0.351 | 0.028 |
| FKSG14 | 0.097 | 0.276 | 0.282 | 0.451 | 0.506 | 0.871 | 0.482 | 0.418 | 0.445 |
| MAD2L1 | −0.144 | −0.146 | −0.462 | 0.119 | 0.085 | 0.805 | 0.081 | 0.283 | 0.036 |
| CDC7 | 0.084 | 0.023 | −0.066 | 0.161 | 0.033 | 0.694 | 0.505 | 0.089 | 0.026 |
| SYBL1 | −0.406 | −0.134 | −0.272 | −0.116 | −0.017 | 0.773 | 0.290 | 0.077 | 0.221 |
| FLJ32803 | −0.199 | 0.261 | 0.160 | 0.064 | 0.137 | 0.829 | 0.374 | 0.107 | 0.125 |
| SIRT1 | 0.091 | 0.113 | −0.037 | 0.398 | 0.267 | 0.587 | 0.436 | 0.280 | 0.037 |
| VBP1 | −0.090 | −0.062 | 0.143 | 0.284 | 0.175 | 0.727 | 0.289 | 0.215 | 0.162 |
| PSMC2 | −0.388 | 0.127 | −0.378 | −0.159 | 0.204 | 0.727 | 0.636 | 0.341 | 0.314 |
| HAT1 | 0.257 | 0.235 | −0.071 | 0.088 | 0.342 | 1.128 | 0.663 | 0.529 | 0.283 |
| NXT2 | −0.270 | 0.066 | 0.139 | 0.163 | 0.048 | 0.588 | 0.512 | 0.228 | 0.136 |
| hIAN2 | −0.152 | 0.029 | −0.118 | 0.419 | 0.071 | 1.107 | 0.267 | 0.243 | −0.083 |
| C10orf22 | 0.224 | 0.085 | 0.537 | 0.433 | 0.375 | 0.893 | 0.362 | 0.388 | 0.213 |
| DKFZp547 | −0.026 | 0.041 | 0.184 | 0.369 | 0.010 | 0.774 | 0.059 | 0.181 | −0.001 |
| LIPA | 0.045 | −0.051 | −0.160 | −0.065 | 0.104 | 0.691 | 0.521 | 0.112 | 0.053 |
| VPS4B | 0.096 | 0.143 | 0.065 | 0.297 | 0.473 | 1.107 | 0.931 | 0.548 | 0.283 |
| SACM1L | −0.113 | 0.012 | 0.097 | 0.200 | 0.058 | 0.584 | 0.697 | 0.247 | −0.001 |
| RAD21 | −0.091 | 0.357 | −0.023 | 0.389 | 0.456 | 0.882 | 0.703 | 0.337 | 0.467 |
| MGC44593 | −0.104 | −0.006 | 0.233 | 0.228 | 0.481 | 0.740 | 0.285 | 0.343 | 0.229 |
| RPGR | 0.118 | 0.283 | 0.227 | 0.469 | 0.216 | 0.774 | 0.383 | 0.246 | 0.162 |
| LOC37672 | 0.219 | 0.278 | 0.166 | 0.621 | 0.151 | 0.647 | 0.402 | 0.194 | 0.241 |
| GNG10 | 0.156 | 0.366 | 0.456 | 0.256 | 0.430 | 0.937 | 0.712 | 0.436 | 0.168 |
| ITM2B | −0.143 | −0.202 | −0.130 | −0.194 | 0.257 | 0.832 | 0.706 | 0.338 | 0.095 |
| SMN1 | 0.090 | 0.182 | 0.258 | 0.157 | 0.142 | 0.856 | 0.824 | 0.358 | 0.083 |
| DYRK1A | −0.141 | −0.061 | −0.020 | −0.044 | 0.301 | 0.832 | 0.674 | 0.298 | 0.216 |
| NUSAP1 | 0.192 | 0.353 | 0.051 | 0.138 | 0.317 | 0.952 | 0.879 | 0.398 | 0.150 |
| SAP30 | 0.165 | 0.013 | 0.186 | 0.209 | 0.042 | 0.926 | 0.733 | 0.275 | 0.047 |
| AUP1 | 0.252 | 0.003 | −0.098 | 0.002 | 0.121 | 0.713 | 0.431 | 0.303 | 0.097 |
| TDE2 | 0.195 | 0.247 | 0.086 | −0.060 | 0.158 | 0.649 | 0.680 | 0.293 | 0.227 |
| DNCL1 | 0.123 | 0.115 | −0.048 | 0.221 | 0.261 | 0.766 | 0.433 | 0.308 | 0.294 |
| TNFAIP8 | 0.300 | 0.120 | 0.426 | 0.539 | 0.295 | 1.015 | 0.536 | 0.267 | 0.222 |
| SNX16 | 0.186 | 0.218 | 0.248 | 0.428 | 0.409 | 0.625 | 0.519 | 0.337 | 0.215 |
| MINPP1 | −0.144 | −0.023 | −0.036 | 0.324 | 0.206 | 0.818 | 0.492 | 0.255 | −0.027 |
| LSM5 | 0.048 | 0.196 | 0.219 | 0.256 | 0.135 | 0.911 | 0.490 | 0.125 | 0.010 |
| PAQR3 | −0.307 | −0.016 | 0.407 | 0.441 | 0.372 | 1.140 | 0.416 | 0.423 | 0.168 |
| RBBP7 | 0.125 | −0.270 | −0.264 | −0.076 | 0.064 | 0.815 | 0.433 | 0.220 | 0.037 |
| C14orf142 | 0.019 | −0.119 | −0.154 | 0.306 | 0.210 | 0.531 | 0.624 | 0.431 | 0.263 |
| HIF1A | 0.292 | 0.214 | −0.166 | −0.202 | 0.169 | 0.697 | 0.362 | 0.253 | −0.009 |
| LOC92912 | −0.007 | 0.236 | 0.048 | 0.300 | 0.355 | 0.599 | 0.840 | 0.406 | 0.115 |
| CLIC4 | −0.369 | −0.173 | −0.015 | 0.060 | 0.396 | 0.549 | 0.661 | 0.223 | 0.115 |
| GPR160 | 0.141 | 0.217 | 0.113 | 0.572 | 0.451 | 0.839 | 0.860 | 0.298 | 0.282 |
| LOC15499 | 0.380 | 0.209 | 0.204 | 0.228 | 0.138 | 0.709 | 0.390 | 0.155 | 0.017 |
| LOC37729 | 0.151 | −0.206 | 0.390 | 0.426 | −0.001 | 0.853 | 0.764 | 0.441 | −0.033 |
| ACSL3 | 0.228 | 0.198 | 0.465 | 0.479 | 0.488 | 0.497 | 0.762 | 0.467 | 0.352 |
| TAF9L | 0.023 | −0.205 | 0.078 | 0.118 | 0.142 | 0.601 | 0.272 | 0.250 | 0.136 |
| LOC20091 | 0.128 | 0.028 | 0.518 | 0.611 | 0.106 | 1.056 | 0.626 | 0.160 | −0.153 |
| HNRPH3 | 0.049 | −0.044 | 0.139 | −0.064 | 0.239 | 0.426 | 0.727 | 0.474 | 0.106 |
| LOC40121 | 0.033 | 0.163 | 0.162 | −0.204 | 0.118 | 0.734 | 0.589 | 0.215 | −0.099 |
| LOC40000 | 0.145 | −0.047 | −0.020 | 0.037 | −0.208 | 0.949 | 0.134 | 0.171 | −0.248 |
| ATP5L | 0.028 | 0.100 | 0.000 | 0.328 | 0.258 | 0.709 | 0.715 | 0.279 | 0.208 |
| SNRPB2 | 0.319 | 0.153 | 0.160 | 0.460 | 0.385 | 0.709 | 0.686 | 0.404 | 0.080 |
| JJAZ1 | −0.175 | 0.038 | 0.150 | 0.468 | 0.145 | 0.694 | 0.398 | 0.328 | −0.040 |
| HNRPK | 0.166 | 0.096 | 0.110 | 0.306 | 0.173 | 0.953 | 0.563 | 0.285 | −0.145 |
| Hs.28465 | 0.329 | 0.236 | 0.589 | 0.635 | 0.580 | 1.069 | 0.879 | 0.447 | 0.117 |
| SMARCA5 | 0.110 | 0.053 | 0.005 | 0.269 | 0.177 | 1.104 | 0.467 | 0.445 | 0.092 |
| OSBPL8 | −0.258 | −0.121 | 0.071 | 0.390 | 0.178 | 1.117 | 0.472 | 0.350 | 0.149 |
| HSPC039 | 0.156 | −0.133 | −0.049 | 0.306 | 0.104 | 0.991 | 0.713 | 0.350 | −0.020 |
| BCAS2 | 0.035 | 0.042 | −0.318 | −0.085 | 0.015 | 0.779 | 0.369 | 0.009 | 0.053 |
| RPL7 | 0.153 | 0.160 | −0.225 | −0.081 | 0.326 | 1.111 | 0.820 | 0.490 | 0.014 |
| FLJ20647 | −0.086 | −0.038 | −0.147 | 0.353 | 0.187 | 0.630 | 0.656 | 0.376 | 0.096 |
| SP3 | 0.324 | 0.399 | 0.700 | 0.432 | 0.524 | 1.104 | 0.592 | 0.637 | 0.343 |
| LOC28555 | −0.099 | 0.048 | 0.009 | −0.056 | 0.012 | 0.913 | 0.571 | 0.251 | 0.011 |
| UBQLN2 | −0.097 | 0.144 | −0.113 | 0.084 | 0.052 | 0.805 | 0.501 | 0.247 | 0.160 |
| SSBP1 | 0.048 | 0.246 | 0.025 | 0.375 | 0.133 | 0.848 | 0.634 | 0.248 | 0.008 |
| SFPQ | 0.220 | 0.322 | 0.392 | 0.404 | 0.205 | 1.219 | 0.900 | 0.324 | 0.256 |
| PGRMC1 | 0.001 | 0.016 | 0.054 | 0.293 | 0.101 | 0.611 | 0.261 | 0.223 | 0.033 |
| TMSL1 | −0.059 | 0.428 | −0.173 | 0.085 | 0.239 | 0.882 | 0.759 | 0.483 | 0.147 |
| LZTFL1 | 0.102 | 0.204 | 0.058 | 0.102 | 0.309 | 0.861 | 0.665 | 0.308 | 0.397 |
| GAS41 | 0.212 | 0.186 | −0.219 | 0.413 | 0.374 | 0.994 | 0.457 | 0.256 | 0.409 |
| KHDRBS1 | 0.115 | 0.184 | −0.174 | 0.241 | 0.245 | 0.637 | 0.494 | 0.268 | 0.223 |
| NOP5/NOP | 0.213 | −0.124 | 0.186 | 0.336 | 0.162 | 1.057 | 0.617 | 0.292 | 0.040 |
| KIN | 0.074 | 0.207 | 0.081 | 0.362 | 0.147 | 1.074 | 0.541 | 0.365 | 0.087 |
| NUCB2 | 0.110 | 0.152 | 0.677 | 0.224 | 0.390 | 1.224 | 0.992 | 0.557 | 0.345 |
| HIG1 | 0.035 | 0.064 | 0.028 | 0.326 | 0.369 | 1.401 | 0.512 | 0.506 | 0.242 |
| RPL9 | 0.101 | −0.298 | 0.129 | 0.174 | 0.245 | 1.361 | 0.988 | 0.712 | −0.046 |
| NAP1L1 | −0.137 | −0.165 | −0.592 | −0.060 | −0.070 | −0.081 | 0.160 | 0.159 | 0.224 |

TABLE 5
Genes screened for pattern set 4 (S3-T4-Pattern-IV). Members of Pattern set 4 are
underlined.

| Gene | 2 nm | 5 nm | 10 nm | 15 nm | 20 nm | 30 nm | 40 nm | 80 nm | 200 nm |
|---|---|---|---|---|---|---|---|---|---|
| LOC12113 | −0.101 | −0.380 | −0.370 | −0.365 | −0.176 | −0.294 | −0.495 | −0.943 | −0.611 |
| SRrp35 | −0.719 | −0.415 | −0.429 | −0.440 | −0.649 | −0.547 | −0.591 | −0.933 | −0.430 |
| CCNB2 | −0.434 | −0.198 | −0.158 | −0.184 | −0.284 | −0.690 | −0.566 | −0.868 | −0.750 |
| HIST1H4D | −0.389 | −0.210 | −0.291 | −0.485 | −0.084 | −0.454 | −0.375 | −0.840 | −0.599 |
| Spc24 | −0.422 | −0.529 | −0.452 | −0.513 | −0.568 | −0.408 | −0.144 | −0.833 | −0.815 |
| MXD3 | −0.132 | −0.219 | −0.097 | −0.356 | −0.230 | −0.431 | −0.438 | −0.805 | −0.612 |
| CD1E | 0.195 | 0.241 | 0.217 | 0.045 | −0.192 | −0.131 | −0.135 | −0.785 | −0.686 |
| RGMA | 0.090 | −0.066 | −0.111 | −0.036 | −0.116 | −0.191 | −0.204 | −0.782 | −0.641 |
| HMGB2 | 0.377 | 0.089 | 0.154 | −0.023 | −0.165 | −0.248 | −0.174 | −0.779 | −0.701 |
| TCTA | −0.491 | −0.088 | −0.239 | −0.347 | −0.557 | −0.477 | −0.346 | −0.770 | −0.645 |
| PTTG2 | −0.111 | −0.193 | −0.102 | −0.291 | −0.474 | −0.484 | −0.354 | −0.736 | −0.689 |
| FLJ23311 | 0.062 | −0.003 | 0.046 | 0.091 | −0.136 | −0.192 | −0.036 | −0.719 | −0.573 |
| MTND6 | 0.298 | 0.324 | 0.301 | 0.081 | 0.052 | −0.082 | −0.012 | −0.715 | −0.581 |
| AURKB | −0.206 | −0.246 | −0.226 | −0.136 | −0.325 | −0.745 | −0.486 | −0.702 | −0.654 |
| ETFB | −0.342 | −0.449 | −0.296 | −0.510 | −0.325 | −0.405 | −0.277 | −0.688 | −0.662 |
| SOSTDC1 | −0.466 | −0.305 | −0.283 | −0.526 | −0.054 | −0.434 | −0.411 | −0.675 | −0.606 |
| TRIM | 0.020 | 0.059 | 0.270 | 0.122 | −0.130 | 0.111 | 0.001 | −0.656 | −0.493 |
| MAGED2 | −0.247 | −0.058 | −0.358 | −0.305 | −0.476 | −0.248 | −0.298 | −0.649 | −0.097 |
| FLJ11029 | −0.459 | −0.411 | −0.337 | −0.343 | −0.092 | −0.452 | −0.351 | −0.644 | −0.520 |
| TPO | −0.403 | −0.333 | −0.244 | −0.285 | −0.278 | −0.436 | −0.306 | −0.638 | −0.471 |
| VEST1 | 0.009 | −0.067 | −0.434 | −0.336 | 0.086 | −0.110 | −0.159 | −0.636 | −0.045 |
| ELOVL4 | 0.343 | 0.215 | 0.331 | 0.264 | 0.242 | 0.314 | 0.268 | −0.635 | −0.479 |
| LOC28644 | −0.205 | −0.108 | −0.244 | −0.137 | −0.043 | −0.083 | −0.240 | −0.617 | −0.416 |
| HRIHFB21 | −0.054 | −0.162 | −0.023 | −0.373 | −0.183 | −0.397 | −0.419 | −0.608 | −0.711 |
| TIRP | −0.202 | −0.150 | −0.285 | −0.360 | −0.684 | −0.276 | −0.246 | −0.569 | −0.605 |
| UBE2J1 | −0.274 | −0.298 | −0.375 | −0.329 | −0.158 | −0.046 | −0.082 | −0.561 | −0.621 |
| FLJ40629 | −0.177 | −0.252 | −0.156 | −0.120 | −0.204 | −0.325 | −0.304 | −0.541 | −0.613 |
| C20orf100 | 0.322 | 0.230 | 0.291 | 0.322 | 0.220 | −0.108 | 0.070 | −0.486 | −0.622 |
| STMN1 | 0.240 | 0.037 | −0.022 | −0.026 | −0.126 | 0.008 | 0.017 | −0.480 | −0.639 |
| GPR56 | −0.200 | −0.258 | −0.213 | −0.170 | −0.126 | −0.120 | −0.116 | −0.434 | −0.689 |
| FLJ20306 | −0.065 | −0.493 | −0.451 | −0.425 | −0.338 | −0.272 | −0.101 | −0.405 | −0.588 |
| MDS1 | −0.279 | −0.209 | 0.017 | −0.296 | −0.231 | −0.329 | −0.383 | −0.232 | −0.741 |
| LOC90317 | 0.613 | 0.223 | 0.252 | 0.296 | 0.198 | 0.090 | 0.002 | 0.275 | 0.229 |
| LOC40047 | 0.376 | 0.471 | 0.467 | 0.427 | 0.533 | 0.355 | 0.498 | 0.300 | 0.609 |
| PARK2 | 0.553 | 0.570 | 0.222 | 0.697 | 0.528 | 0.700 | 0.741 | 0.321 | 0.401 |
| ELA3A | 0.354 | 0.537 | 0.323 | 0.490 | 0.322 | 0.255 | 0.315 | 0.332 | 0.632 |
| C6orf145 | 0.428 | 0.376 | 0.577 | 0.485 | 0.516 | 0.495 | 0.598 | 0.377 | 0.218 |
| SPESP1 | 0.410 | 0.495 | 0.409 | 0.529 | 0.283 | 0.662 | 0.348 | 0.377 | 0.156 |
| NRP2 | 0.524 | 0.504 | 0.321 | 0.576 | 0.254 | 0.234 | 0.541 | 0.377 | 0.669 |
| HMGCS1 | −0.093 | 0.588 | 0.385 | 0.365 | 0.295 | 0.166 | 0.216 | 0.391 | 0.621 |
| LOC38902 | 0.178 | 0.105 | 0.018 | −0.055 | 0.196 | 0.075 | 0.229 | 0.443 | 0.586 |
| ZNF90 | 0.517 | 0.359 | 0.479 | 0.642 | 0.556 | 0.538 | 0.592 | 0.472 | 0.340 |
| KIR3DL3 | 0.498 | 0.518 | 0.486 | 0.477 | 0.681 | 0.651 | 0.397 | 0.474 | 0.849 |
| ALDOC | 0.611 | 0.652 | 0.697 | 0.841 | 0.652 | 0.317 | 0.415 | 0.495 | 0.585 |
| FLJ10439 | −0.274 | 0.095 | 0.050 | −0.014 | −0.005 | 0.040 | 0.051 | 0.507 | 0.609 |
| Hs.519802 | 0.651 | 0.459 | −0.111 | 0.428 | −0.028 | 0.405 | 0.235 | 0.520 | 0.607 |
| KIAA0907 | 0.507 | 0.315 | 0.297 | 0.321 | 0.359 | 0.343 | 0.013 | 0.541 | 0.602 |
| Hs.498571 | 0.704 | 0.491 | 0.601 | 0.637 | 0.598 | 0.563 | 0.385 | 0.541 | 0.606 |
| INA | 0.587 | 0.357 | 0.245 | 0.296 | 0.059 | 0.212 | 0.077 | 0.546 | 0.567 |
| C20orf97 | 0.154 | 0.424 | 0.444 | 0.324 | 0.389 | 0.169 | 0.247 | 0.553 | 0.642 |
| LOC33891 | 0.123 | 0.599 | 0.554 | 1.043 | 0.446 | 0.701 | 0.264 | 0.557 | 0.512 |
| DNAJA1 | 0.527 | 0.113 | 0.136 | 0.077 | 0.014 | 0.045 | −0.074 | 0.564 | 0.606 |
| SPR | −0.538 | −0.096 | −0.501 | −0.099 | −0.282 | −0.171 | −0.152 | 0.567 | 0.660 |
| LOC15905 | 0.255 | 0.054 | −0.360 | −0.212 | 0.046 | 0.011 | −0.083 | 0.578 | 0.648 |
| FKBP4 | 0.009 | −0.278 | −0.356 | −0.500 | −0.373 | −0.810 | −0.613 | 0.581 | 0.726 |
| KIAA0433 | 0.437 | 0.791 | 0.481 | 0.457 | 0.595 | 0.485 | 0.649 | 0.591 | 0.427 |
| EGFL9 | 0.417 | 0.245 | 0.538 | 0.514 | 0.391 | 0.386 | 0.508 | 0.606 | 0.697 |
| GAB2 | 0.582 | 0.442 | 0.479 | 0.412 | 0.494 | 0.226 | 0.417 | 0.609 | 0.486 |
| BIK | 0.113 | −0.106 | 0.154 | 0.245 | 0.395 | 0.371 | 0.221 | 0.614 | 0.577 |
| LOC91942 | 0.635 | 0.116 | 0.110 | 0.108 | 0.139 | 0.400 | 0.474 | 0.617 | 0.550 |
| FLJ20449 | 0.564 | 0.365 | 0.443 | 0.236 | 0.249 | 0.165 | 0.164 | 0.619 | 0.370 |
| Hs.163734 | 0.377 | −0.096 | −0.089 | 0.051 | 0.179 | 0.150 | 0.142 | 0.622 | 0.624 |
| HMGCR | 0.353 | 0.454 | 0.588 | 0.618 | 0.617 | 0.605 | 0.676 | 0.624 | 0.488 |
| AMSH-LP | 0.072 | −0.030 | −0.185 | −0.075 | −0.008 | 0.099 | 0.265 | 0.624 | 0.641 |
| HSPE1 | −0.173 | 0.091 | 0.058 | 0.163 | −0.023 | −0.280 | −0.125 | 0.625 | 0.570 |
| PDGFA | 0.322 | 0.224 | 0.327 | 0.174 | 0.198 | 0.099 | 0.007 | 0.627 | 0.636 |
| PSME3 | −0.060 | −0.120 | −0.108 | 0.201 | 0.082 | 0.189 | 0.159 | 0.629 | 0.579 |
| HAGHL | 0.367 | 0.562 | 0.423 | 0.380 | 0.358 | 0.063 | 0.094 | 0.636 | 0.550 |
| C9orf58 | 0.354 | 0.372 | 0.406 | 0.415 | 0.425 | 0.264 | 0.547 | 0.655 | 0.541 |
| FCGR1A | 0.442 | 0.630 | 0.364 | 0.170 | 0.693 | 0.504 | 0.015 | 0.661 | 0.589 |
| COTL1 | 0.413 | 0.132 | 0.190 | 0.141 | 0.109 | 0.036 | 0.116 | 0.666 | 0.650 |
| FLJ12519 | 0.114 | 0.084 | 0.076 | 0.220 | 0.308 | 0.265 | 0.248 | 0.671 | 0.493 |
| BMP4 | −0.129 | −0.035 | −0.106 | −0.149 | 0.078 | 0.022 | 0.143 | 0.673 | 0.673 |
| MAN1A1 | 0.085 | 0.503 | 0.243 | 0.473 | 0.599 | 0.488 | 0.631 | 0.682 | 0.676 |
| ITGB1BP3 | 0.729 | 0.718 | 0.734 | 0.656 | 0.774 | 0.564 | 0.453 | 0.683 | 0.499 |
| MRPL18 | −0.628 | −0.595 | −0.466 | −0.219 | 0.108 | 0.503 | 0.409 | 0.686 | 0.711 |
| LOC40166 | 0.557 | 0.516 | 0.336 | 0.447 | 0.359 | 0.375 | 0.586 | 0.694 | 0.430 |
| ATF7IP2 | 0.378 | 0.126 | 0.263 | 0.232 | 0.190 | 0.077 | 0.032 | 0.726 | 0.823 |
| EEF1E1 | 0.315 | 0.144 | 0.163 | 0.424 | 0.344 | 0.512 | 0.474 | 0.753 | 0.795 |
| FLJ20485 | 0.036 | 0.262 | 0.336 | 0.016 | 0.111 | 0.041 | 0.214 | 0.757 | 0.765 |
| CGI-01 | 0.121 | 0.308 | 0.138 | 0.203 | 0.227 | 0.146 | 0.127 | 0.768 | 0.718 |
| Hs.489117 | 0.364 | 0.350 | 0.093 | 0.313 | 0.333 | 0.455 | 0.501 | 0.800 | 0.768 |
| HSPCB | 0.010 | −0.089 | −0.185 | −0.093 | 0.068 | −0.181 | −0.395 | 0.805 | 0.759 |
| LRRFIP1 | 0.313 | 0.021 | 0.004 | 0.121 | 0.614 | 0.953 | 0.706 | 0.817 | 0.820 |
| HSPD1 | −0.099 | −0.181 | −0.404 | −0.086 | 0.129 | 0.262 | 0.083 | 0.833 | 0.771 |
| MGC2494 | 0.507 | 0.178 | 0.236 | 0.251 | 0.271 | −0.089 | 0.073 | 0.845 | 0.812 |
| HSPA8 | 0.320 | 0.163 | 0.117 | 0.058 | −0.127 | −0.136 | −0.127 | 0.848 | 0.916 |
| HSPA1A | −0.020 | 0.106 | 0.063 | −0.039 | 0.096 | 0.184 | 0.130 | 0.864 | 0.432 |
| MYCN | 0.401 | 0.535 | 0.599 | 0.687 | 0.573 | 0.697 | 0.558 | 0.866 | 0.878 |
| Hs.448642 | −0.278 | −0.321 | −1.110 | 0.064 | 0.032 | 1.154 | 0.973 | 0.896 | 0.963 |
| MGC2408 | −0.006 | 0.038 | −0.021 | 0.059 | 0.230 | 0.019 | −0.005 | 0.916 | 0.873 |
| FTL | 0.348 | 0.131 | 0.080 | 0.157 | 0.207 | 0.421 | 0.385 | 0.920 | 0.832 |
| HSPCA | −0.174 | 0.063 | −0.037 | 0.155 | 0.046 | −0.039 | −0.061 | 0.959 | 0.954 |
| FLJ32942 | 0.162 | −0.037 | 0.042 | 0.064 | 0.028 | −0.076 | 0.125 | 0.968 | 0.875 |
| ATF5 | 1.060 | 0.858 | 0.879 | 0.930 | 0.847 | 0.331 | 0.588 | 0.983 | 0.894 |
| LOC14891 | 0.558 | 0.286 | 0.172 | 0.197 | 0.130 | 0.221 | 0.289 | 0.993 | 0.947 |
| HSPH1 | 0.226 | 0.153 | 0.126 | 0.363 | 0.453 | 0.356 | 0.310 | 1.019 | 0.973 |
| MYC | 0.367 | 0.419 | 0.048 | 0.397 | 0.199 | 0.326 | 0.343 | 1.125 | 1.046 |
| LOC12113 | −0.637 | −0.486 | −0.348 | −0.651 | −0.646 | 0.058 | −0.616 | −0.834 | −0.431 |
| SRrp35 | −0.561 | −0.363 | −0.362 | −0.582 | −0.710 | −0.665 | −0.539 | −0.244 | −0.942 |
| CCNB2 | −0.123 | −0.018 | −0.345 | −0.048 | 0.051 | −0.047 | −0.129 | −0.739 | −0.859 |
| HIST1H4D | −0.122 | −0.104 | 0.167 | −0.116 | 0.018 | 0.003 | −0.140 | −1.273 | −0.168 |
| Spc24 | −0.244 | −0.284 | −0.200 | −0.150 | −0.194 | −0.130 | −0.108 | −0.969 | −0.474 |
| MXD3 | −0.461 | −0.099 | −0.176 | −0.042 | 0.074 | 0.104 | −0.262 | −0.807 | −0.276 |
| CD1E | 0.434 | 0.179 | 0.055 | −0.039 | −0.056 | 0.082 | 0.034 | −0.187 | −1.015 |
| RGMA | −0.117 | −0.242 | −0.144 | −0.157 | −0.176 | −0.431 | −0.453 | −0.727 | −0.432 |
| HMGB2 | 0.041 | −0.034 | −0.174 | −0.139 | −0.239 | −0.200 | −0.174 | −0.381 | −0.915 |
| TCTA | −0.221 | −0.581 | −0.458 | −0.063 | −0.161 | −0.010 | −0.478 | −0.367 | −0.304 |
| PTTG2 | −0.459 | −0.214 | −0.315 | 0.032 | −0.076 | −0.124 | −0.143 | −0.711 | −0.403 |
| FLJ23311 | −0.179 | −0.225 | −0.370 | −0.178 | −0.138 | −0.011 | 0.107 | −0.591 | −0.602 |
| MTND6 | −0.110 | −0.031 | −0.099 | −0.111 | 0.004 | 0.049 | 0.085 | −0.738 | −0.321 |
| AURKB | −0.241 | −0.222 | −0.231 | −0.134 | −0.053 | −0.041 | −0.165 | −0.502 | −0.720 |
| ETFB | −0.313 | −0.389 | −0.304 | −0.215 | −0.101 | −0.086 | −0.163 | −0.613 | −0.278 |
| SOSTDC1 | −0.162 | −0.518 | −0.569 | −0.367 | −0.460 | −0.438 | −0.468 | −0.380 | −0.641 |
| TRIM | 0.129 | 0.098 | −0.108 | −0.028 | −0.005 | 0.129 | −0.106 | −0.560 | −0.765 |
| MAGED2 | 0.022 | −0.091 | −0.131 | −0.447 | −0.365 | −0.375 | −0.377 | −0.177 | −0.669 |
| FLJ11029 | −0.200 | −0.004 | −0.049 | 0.052 | 0.058 | −0.045 | 0.082 | −0.657 | −0.323 |
| TPO | −0.251 | −0.125 | −0.125 | −0.111 | −0.045 | −0.450 | −0.059 | −0.897 | −0.404 |
| VEST1 | −0.312 | −0.616 | −0.278 | −0.194 | −0.213 | −0.293 | −0.258 | −0.135 | −0.692 |
| ELOVL4 | 0.053 | −0.020 | 0.126 | −0.057 | −0.163 | −0.209 | −0.079 | −0.780 | −0.404 |
| LOC28644 | −0.367 | −0.156 | −0.359 | −0.452 | −0.046 | −0.181 | −0.590 | −0.694 | −0.506 |
| HRIHFB21 | 0.058 | −0.120 | −0.091 | −0.225 | −0.225 | −0.262 | −0.243 | −0.935 | −0.251 |
| TIRP | −0.465 | −0.636 | −0.374 | −0.207 | −0.225 | −0.312 | −0.375 | −0.389 | −0.638 |
| UBE2J1 | −0.255 | 0.079 | −0.031 | −0.061 | −0.136 | 0.050 | −0.232 | −1.074 | −0.697 |
| FLJ40629 | −0.272 | −0.394 | −0.235 | −0.101 | −0.098 | −0.223 | −0.186 | −0.783 | −0.562 |
| C20orf100 | −0.210 | 0.073 | −0.148 | −0.117 | 0.188 | 0.221 | 0.052 | −0.654 | −0.594 |
| STMN1 | 0.035 | −0.003 | −0.049 | −0.053 | −0.033 | −0.019 | −0.035 | −0.548 | −0.586 |
| GPR56 | −0.072 | −0.137 | −0.077 | −0.119 | −0.478 | −0.383 | −0.436 | −0.683 | −0.522 |
| FLJ20306 | −0.244 | −0.370 | −0.186 | −0.105 | −0.417 | −0.474 | −0.320 | −0.317 | −0.640 |
| MDS1 | −0.468 | −0.320 | −0.475 | −0.214 | −0.358 | −0.281 | −0.710 | −0.338 | −0.320 |
| LOC90317 | −0.081 | 0.151 | 0.081 | 0.038 | 0.094 | −0.014 | −0.133 | 0.644 | 0.611 |
| LOC40047 | 0.484 | 0.390 | 0.532 | 0.737 | 0.494 | −0.165 | 0.402 | 0.153 | 0.448 |
| PARK2 | 0.397 | 0.233 | 0.468 | 0.581 | 0.315 | 0.351 | 0.470 | 0.878 | 0.431 |
| ELA3A | 0.309 | 0.543 | 0.299 | 0.418 | 0.614 | 0.402 | 0.364 | 0.357 | 0.311 |
| C6orf145 | 0.388 | 0.511 | 0.361 | 0.170 | 0.213 | 0.153 | 0.431 | 0.637 | 0.502 |
| SPESP1 | 0.293 | 0.053 | 0.156 | 0.519 | 0.354 | 0.366 | 0.478 | 0.604 | 0.438 |
| NRP2 | 0.597 | 0.352 | 0.420 | 0.508 | 0.195 | 0.488 | 0.136 | 0.533 | 0.455 |
| HMGCS1 | 0.869 | 0.481 | 0.432 | 0.287 | 0.340 | 0.372 | 0.148 | 1.435 | 0.122 |
| LOC38902 | 0.259 | 0.045 | 0.128 | 0.013 | −0.103 | 0.151 | −0.150 | 0.277 | 0.717 |
| ZNF90 | 0.767 | 0.328 | 0.293 | 0.509 | 0.242 | 0.295 | 0.165 | 0.603 | 0.507 |
| KIR3DL3 | 0.465 | 0.331 | 0.433 | 0.371 | 0.479 | 0.295 | 0.405 | 0.411 | 0.685 |
| ALDOC | 0.198 | 0.451 | 0.391 | 0.514 | 0.633 | 0.577 | 0.599 | 0.719 | 0.680 |
| FLJ10439 | 0.211 | 0.222 | 0.201 | 0.233 | 0.247 | 0.312 | 0.478 | 0.843 | 0.691 |
| Hs.519802 | 0.616 | 0.428 | 0.360 | 0.319 | 0.040 | 0.255 | 0.248 | 0.616 | 0.124 |
| KIAA0907 | 0.163 | 0.219 | 0.169 | 0.102 | 0.144 | 0.203 | 0.252 | 0.728 | 0.517 |
| Hs.498571 | 0.316 | 0.570 | 0.538 | 0.570 | 0.566 | 0.370 | 0.499 | 0.637 | 0.601 |
| INA | 0.086 | 0.104 | 0.017 | 0.025 | 0.141 | 0.074 | 0.238 | 0.724 | 0.751 |
| C20orf97 | −0.052 | 0.099 | 0.033 | −0.036 | 0.136 | 0.088 | −0.013 | 0.624 | 0.518 |
| LOC33891 | 0.385 | 0.458 | 0.349 | 0.422 | 0.342 | 0.355 | 0.433 | 0.869 | 0.951 |
| DNAJA1 | 0.443 | 0.192 | 0.138 | 0.047 | −0.048 | 0.081 | 0.136 | 0.739 | 0.003 |
| SPR | −0.042 | 0.113 | −0.028 | 0.240 | 0.025 | −0.078 | 0.217 | 0.854 | 1.111 |
| LOC15905 | −0.065 | 0.251 | 0.197 | 0.330 | 0.310 | 0.067 | 0.233 | 0.241 | 0.670 |
| FKBP4 | 0.028 | −0.096 | −0.312 | −0.087 | 0.055 | −0.067 | 0.160 | 1.018 | 0.858 |
| KIAA0433 | 0.548 | 0.501 | 0.571 | 0.421 | 0.613 | 0.460 | 0.446 | 0.597 | 0.683 |
| EGFL9 | 0.396 | 0.269 | 0.342 | 0.295 | 0.318 | 0.463 | 0.167 | 0.748 | 0.969 |
| GAB2 | −0.009 | −0.057 | −0.021 | −0.022 | −0.015 | 0.086 | 0.142 | 0.705 | 0.717 |
| BIK | 0.189 | 0.255 | 0.353 | 0.247 | 0.241 | 0.232 | 0.036 | 0.630 | 0.685 |
| LOC91942 | 0.092 | 0.094 | 0.361 | 0.193 | −0.020 | 0.006 | 0.173 | 0.278 | 0.648 |
| FLJ20449 | 0.474 | 0.654 | 0.409 | 0.374 | 0.514 | 0.330 | 0.289 | 0.369 | 0.239 |
| Hs.163734 | 0.104 | 0.351 | 0.229 | 0.048 | 0.071 | 0.033 | −0.092 | 0.562 | 1.220 |
| HMGCR | 0.418 | 0.317 | 0.464 | 0.376 | 0.410 | 0.308 | 0.389 | 0.750 | 0.646 |
| AMSH-LP | 0.173 | 0.177 | 0.149 | 0.134 | 0.116 | −0.057 | −0.130 | 0.769 | 0.472 |
| HSPE1 | 0.030 | 0.136 | 0.058 | 0.178 | 0.152 | 0.125 | 0.205 | 0.725 | 0.830 |
| PDGFA | 0.059 | 0.104 | 0.020 | −0.099 | 0.062 | −0.044 | −0.591 | 0.458 | 0.388 |
| PSME3 | 0.294 | 0.109 | 0.281 | 0.107 | 0.042 | 0.094 | 0.094 | 0.632 | 0.249 |
| HAGHL | −0.123 | −0.073 | −0.089 | −0.039 | 0.052 | 0.015 | 0.060 | 0.932 | 0.523 |
| C9orf58 | 0.142 | 0.237 | 0.302 | 0.197 | 0.189 | 0.409 | 0.292 | 0.401 | 0.730 |
| FCGR1A | 0.837 | 0.450 | 0.665 | 0.363 | 0.195 | 0.502 | 0.457 | 0.630 | 0.421 |
| COTL1 | 0.127 | 0.044 | 0.065 | 0.054 | −0.011 | 0.041 | 0.011 | 0.699 | 0.426 |
| FLJ12519 | −0.071 | 0.068 | 0.236 | 0.006 | −0.080 | −0.009 | −0.102 | 0.446 | 0.632 |
| BMP4 | 0.002 | 0.062 | 0.078 | 0.085 | −0.190 | −0.086 | −0.265 | 0.778 | 0.278 |
| MAN1A1 | 0.557 | 0.588 | 0.598 | 0.554 | 0.487 | 0.260 | 0.365 | 0.403 | 0.618 |
| ITGB1BP3 | 0.499 | 0.747 | 0.818 | 0.731 | 0.288 | 0.671 | 0.665 | 0.625 | 1.080 |
| MRPL18 | 0.267 | 0.426 | 0.412 | 0.549 | 0.413 | 0.376 | 0.424 | 0.664 | 0.905 |
| LOC40166 | 0.340 | 0.267 | 0.445 | 0.870 | 0.219 | 0.430 | 0.518 | 0.528 | 0.694 |
| ATF7IP2 | 0.213 | 0.281 | 0.245 | 0.156 | 0.004 | 0.222 | −0.408 | 0.680 | 0.796 |
| EEF1E1 | 0.248 | 0.116 | 0.470 | 0.183 | −0.036 | 0.130 | 0.115 | 0.493 | 0.679 |
| FLJ20485 | 0.363 | 0.421 | 0.117 | −0.039 | 0.054 | 0.109 | 0.291 | 0.982 | 0.130 |
| CGI-01 | 0.241 | 0.090 | 0.086 | 0.011 | 0.013 | 0.003 | 0.022 | 0.905 | 0.702 |
| Hs.489117 | 0.279 | 0.407 | 0.397 | 0.370 | 0.135 | 0.209 | 0.298 | 0.512 | 1.167 |
| HSPCB | 0.128 | 0.174 | −0.008 | 0.095 | 0.181 | 0.066 | 0.382 | 1.060 | 0.810 |
| LRRFIP1 | 0.634 | 0.783 | 0.930 | 0.514 | 0.354 | 0.341 | 0.614 | 0.395 | 0.657 |
| HSPD1 | 0.370 | 0.167 | 0.213 | 0.111 | 0.009 | 0.095 | 0.157 | 1.017 | 0.495 |
| MGC2494 | −0.120 | 0.079 | 0.031 | −0.054 | 0.032 | 0.027 | 0.080 | 1.149 | 1.398 |
| HSPA8 | 0.172 | 0.009 | −0.067 | −0.087 | −0.199 | −0.030 | 0.058 | 1.018 | 0.553 |
| HSPA1A | −0.095 | 0.319 | −0.014 | 0.307 | −0.092 | −0.042 | 0.064 | 0.708 | 0.763 |
| MYCN | 0.526 | 0.393 | 0.470 | 0.483 | 0.455 | 0.282 | 0.150 | 0.670 | 0.526 |
| Hs.448642 | 0.594 | 0.843 | 1.029 | 1.090 | 0.791 | 0.719 | 0.847 | 0.108 | 1.003 |
| MGC2408 | −0.025 | 0.334 | 0.091 | 0.062 | 0.007 | −0.045 | 0.170 | 0.608 | 0.708 |
| FTL | 0.033 | 0.193 | 0.306 | 0.230 | 0.166 | 0.182 | 0.122 | 0.416 | 0.602 |
| HSPCA | 0.479 | 0.348 | 0.092 | 0.214 | 0.070 | 0.254 | 0.331 | 1.124 | 0.416 |
| FLJ32942 | 0.002 | −0.050 | 0.113 | −0.050 | −0.076 | −0.043 | 0.128 | 1.068 | 1.378 |
| ATF5 | 0.231 | 0.212 | 0.225 | 0.370 | 0.292 | 0.383 | 0.559 | 1.447 | 1.194 |
| LOC14891 | 0.260 | 0.354 | 0.257 | 0.336 | 0.229 | 0.492 | 0.454 | 0.787 | 0.924 |
| HSPH1 | 0.491 | 0.518 | 0.350 | 0.324 | 0.304 | 0.205 | 0.267 | 0.947 | 0.757 |
| MYC | 0.389 | 0.088 | −0.030 | −0.108 | −0.310 | −0.137 | 0.033 | 0.751 | 0.513 |
| RGS16 | 0.238 | 0.164 | 0.231 | −0.001 | −0.055 | 0.070 | −0.033 | 1.797 | 1.883 |
| LOC37796 | 0.198 | 0.335 | 0.256 | 0.342 | 0.181 | 0.436 | 0.325 | 0.911 | 0.985 |
| DNAJB1 | −0.125 | 0.031 | −0.142 | −0.057 | −0.112 | 0.012 | −0.116 | 1.344 | 1.270 |
| HSPA1B | −0.006 | −0.022 | −0.207 | −0.304 | −0.119 | −0.274 | −0.328 | 1.598 | 1.300 |

In various methods described herein, molecular and cellular responses of cells treated with nanoparticles of particular or of varying sizes are examined. Whole-genome gene expression measurements can be examined (e.g., as described above) to identify size-dependent effects in response to nanoparticles. In certain embodiments the biological response can be easily categorized by size of the nanoparticle, such as either below 5 nm or above 80 nm. There are also genes that are down-regulated in proportion to nanoparticle size, representing “linear scaling effects”. In addition, a cluster of genes can be differentially regulated by 20-40 nm nanoparticle treated cells in a time-persistent pattern. In addition, biological effects other than size-dependent effects can be identified as described herein.

Where an effect is determined to be size dependent, modifiation or elimination of that effect is expected to require use of a different size nanoparticle. Where an effect is determined not to be size-dependent, alteration or elimination of that effect expected to require a change in the nanoparaticular composition or the composition of pharmaceuticals or other reagents associated with, adhered to, or incorporated in the nanoparticle(s).

In certain embodiments, gene function, promoter, and pathway analyses are performed to reveal differential signaling responses that are correlated to nanoparticle size ranges of 2-10 nm, 20-40 nm, and 80-200 nm.

In certain embodiments, cellular responses are measured using Jurkat cells or other human or non-human mammalian cells, or bacterial cells, or protozoan cells, etc. In other embodiments, other types of cells or animal models are used to test specific size-dependent effect on a particular tissue or animal. For examples, cells from other types of tissues include but are not limited to, liver (i.e., hepatocytes), kidney, cardiovascular, epithelial, primary neurons, keratinocytes, fibroblasts, embryonic cells, lung fibroblasts, lung epithelial, peripheral blood, lymphocytes, intestinal, coroneal, placental. These tissues can help show the size-dependent biological effect of any nanoparticle in a particular tissue to show the effect these particles will have if the cross the blood brain barrier (BBB), how they may affect food absorption in the gut, or the effect on the endocrine system. In another embodiment, animal or microbial models are used to test cellular response after treatment of nanoparticles, including, monkey, rabbit, dog mouse, C. elegans, fruitfly, daphnia, etc. In certain embodiments, the cellular response experiments are carried out in three-dimensional cell cultures. In certain embodiments the cells are contacted by administering nanoparticles (e.g., orally, rectally, nasally, intravenously, transdermally, etc.) to a test organism, preferably a non-human mammal.

### II. Assays for Expression of Levels of Genes as Indicators of Size-Dependent or Size-Independent Nanoparticle Effects.

In certain embodiments this invention identifies a number of genes, altered expression (e.g., upregulation or downregulation) of which provides an indication of size-dependent or nanoparticle effects. In various embodiments the expression levels of one or more, two or more, 5 or more, 10 or more, 20 or more, etc., or all of the genes of pattern set 1, and/or pattern set 2, and/or pattern set 3, and/or pattern set 4 are determined.

Expression levels of a gene can be altered by changes in the copy number of the gene and/or transcription of the gene product (i.e., transcription of mRNA), and/or by changes in translation of the gene product (i.e., translation of the protein), and/or by post-translational modification(s) (e.g. protein folding, glycosylation, etc.). Thus, in various embodiments, assays of this invention typically involve assaying for level of transcribed mRNA (or other nucleic acids expressed by the genes identified herein), or level of translated protein, etc. Examples of such approaches are described below.

A) Nucleic-Acid Based Assays.

1. Target Molecules.

Changes in expression level can be detected by measuring changes in mRNA and/or a nucleic acid derived from the mRNA (e.g. reverse-transcribed cDNA, etc.). In order to measure gene expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes. Typically the sample is derived from a cell, tissue, or organism contacted with one or more types of nanoparticle.

The nucleic acid (e.g., mRNA, or nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In certain embodiments, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In certain embodiments, where it is desired to quantify the transcription level (and thereby expression) of factor(s) of interest in a sample, the nucleic acid sample is one in which the concentration of the nucleic acids in the sample, is proportional to the transcription level (and therefore expression level) of the gene(s) of interest. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript, or large differences or changes in nucleic acid concentration are desired, no elaborate control or calibration is required.

In the simplest embodiment, the nucleic acid sample is the total mRNA or a total cDNA isolated and/or otherwise derived from a biological sample (e.g., a sample from a neural cell or tissue). The nucleic acid may be isolated from the sample according to any of a number of methods well known to those of skill in the art as indicated above.

2. Hybridization-Based Assays.

Using the known sequence(s) of the various genes identified in pattern set 1, pattern set, pattern set 3, and/or pattern set 4, and/or in Table 2, and/or Table 3, and/or Table 4, and/or Table 5 detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed mRNA), typically fragmented and separated on an electrophoretic gel, is hybridized to a probe specific for the target nucleic acid. Comparison of the intensity of the hybridization signal from the target specific probe with a “control” probe (e.g. a probe for a “housekeeping gene) provides an estimate of the relative expression level of the target nucleic acid.

Alternatively, the mRNA transcription level can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes can be used to identify and/or quantify the target mRNA. Appropriate controls (e.g. probes to housekeeping genes) can provide a reference for evaluating relative expression level.

An alternative means for determining the gene expression level(s) is in situ hybridization. In situ hybridization assays are well known (e.g., Angerer (1987) Meth. Enzymol 152: 649). Generally, in situ hybridization comprises the following major steps:

(1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use can vary depending on the particular application.

In some applications it is necessary to block the hybridization capacity of repetitive sequences. Thus, in some embodiments, tRNA, human genomic DNA, or Cot-1 DNA is used to block non-specific hybridization.

3. Amplification-Based Assays.

In another embodiment, amplification-based assays can be used to measure expression of one or more of the genes described herein. In such amplification-based assays, the target nucleic acid sequences (e.g., genes upregulated or downregulated by nanoparticle exposure) act as template(s) in amplification reaction(s) (e.g. Polymerase Chain Reaction (PCR), reverse-transcription PCR (RT-PCR), etc.). In a quantitative amplification, the amount of amplification product will be proportional to the amount of template in the original sample. Comparison to appropriate controls (e.g., similar measurements made for samples from healthy mammals) provides a measure of the transcript level.

Methods of “quantitative” amplification are well known to those of skill in the art. For example, in certain embodiments, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.).

One illustrative internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The nucleic acid sequence(s) provided herein are sufficient to enable one of skill to routinely select primers to amplify any portion of the gene(s).

In certain embodiments, the expression levels can be determined using a real-time PCR assay. Real-time polymerase chain reaction, also called quantitative real time polymerase chain reaction (QRT-PCR) or kinetic polymerase chain reaction, is a technique based on polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample. The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-strand DNA, and modified DNA oligonucleotide probes that fluoresce when hybridized with a complementary DNA. Frequently, real-time polymerase chain reaction is combined with reverse transcription polymerase chain reaction to quantify low abundance messenger RNA (mRNA), enabling a researcher to quantify relative gene expression at a particular time, or in a particular cell or tissue type.

Real-time PCR using double-stranded DNA dyes involves the use of a DNA-binding dye (e.g., SYBR Green) that binds to all double-stranded (ds)DNA in a PCR reaction, causing fluorescence of the dye. An increase in DNA product during PCR therefore leads to an increase in fluorescence intensity and is measured at each cycle, thus allowing DNA concentrations to be quantified. However, dsDNA dyes such as SYBR green bind to all dsDNA PCR products, including nonspecific PCR products (“primer dimers”). This can potentially interfere with or prevent accurate quantification of the intended target sequence. When using DNA-binding dyes, the PCR reaction is typically prepared as usual, with the addition of the fluorescent dsDNA dye. The reaction is run in a thermocycler, and after each cycle, the levels of fluorescence are measured with a detector; the dye only fluoresces when bound to the dsDNA (i.e., the PCR product). With reference to a standard dilution, the dsDNA concentration in the PCR can be determined.

Like other real-time PCR methods, the values obtained do not have absolute units associated with it (i.e. mRNA copies/cell). A comparison of a measured DNA/RNA sample to a standard dilution gives a fraction or ratio of the sample relative to the standard, allowing relative comparisons between different tissues, samples, or experimental conditions. To ensure accuracy in the quantification, it is usually necessary to normalize expression of a target gene to a stably expressed gene (see below). This can correct possible differences in RNA quantity or quality across experimental samples.

Using fluorescent reporter probes is the most accurate and most reliable of the methods. This approach uses a sequence-specific RNA or DNA-based probe (e.g., one or more probes complementary to the amplification product(s)) to quantify only the DNA containing the probe sequence. Use of the reporter probe thus significantly increases specificity, and allows quantification even in the presence of some non-specific DNA amplification. Reporter probe real-time PCR methods are commonly carried out with an RNA- or DNA-probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter. When using fluorescent probes, the PCR reaction is typically prepared as usual, and the reporter probe is added. As the reaction commences, during the annealing stage of the PCR both probe and primers anneal to the DNA target. Polymerization of a new DNA strand is initiated from the primers, and once the polymerase reaches the probe, its 5′-3-exonuclease degrades the probe, physically separating the fluorescent reporter from the quencher, resulting in an increase in fluorescence. Fluorescence is detected and measured in the real-time PCR thermocycler, and its geometric increase corresponding to exponential increase of the product is used to determine the threshold cycle (CT) in each reaction.

In one approach to quantifying real-time PCR, relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Ct. Since the quantity of DNA doubles every cycle during the exponential phase, relative amounts of DNA can be calculated, e.g. a sample whose Ct is 3 cycles earlier than another's has 2³=8 times more template.

Amounts of RNA or DNA can then be determined by comparing the results to a standard curve produced by RT-PCR of serial dilutions (e.g. undiluted, 1:4, 1:16, 1:64) of a known amount of RNA or DNA. As mentioned above, to accurately quantify gene expression, the measured amount of RNA from the gene of interest is divided by the amount of RNA from a housekeeping gene measured in the same sample to normalize for possible variation in the amount and quality of RNA between different samples. This normalization permits accurate comparison of expression of the gene of interest between different samples, provided that the expression of the reference (housekeeping) gene used in the normalization is very similar across all the samples. Methods of performing quantitative real-time PCR are well known to those of skill in the art (see, e.g. Dorak (2006) Real Time PCR (BIOS Advanced Methods), Taylor & Francis, New York; Edwards (2004) Real-Time PCR: An Essential Guide, Taylor & Francis, New York; King and O'Connell (2002) RT-PCR Protocols (Methods in Molecular Biology), Humana Press, Totowa, N.J., and the like).

4. Hybridization Formats and Optimization of Hybridization

a. Array-Based Hybridization Formats

In certain embodiments, the methods of this invention can be utilized in array-based hybridization formats. Arrays typically comprise a multiplicity of different “probe” or “target” nucleic acids (or other compounds) attached to one or more surfaces (e.g., solid, membrane, or gel). In certain embodiments, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Global gene expression profiles of cells treated with nanoparticles to identify particle size-dependent molecular responses can be performed using such any gene expression array platform. In certain embodiments, the gene expression array platform used is an Affymetrix microarray or Illumina microarray, e.g., as described in Barnes et al. (2005) Nucleic Acids Res 33: 5914-5923. Other suitable microarray platforms include but are not limited to, arrays available from Combimatrix, Agilent, NimbleGen, etc.

Arrays, particularly nucleic acid arrays, can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934. In addition, a number of high density arrays are commercially available.

b. Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario), Q Beta Replicase systems, or branched DNA amplifier technology commercialized by Panomics, Inc. (Fremont Calif.), and the like.

c. Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results, and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In certain embodiments, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

B) Polypeptide-Based Assays.

In various embodiments the peptide(s) encoded by one or more genes listed in pattern set, and/or pattern set2, and/or pattern set 3, and/or pattern set 4, and/or Table 1, and/or Table 2, and/or Table 3, and/or Table 4, and/or Table 5 can be detected and quantified to provide a measure of expression level. Protein expression can be measured by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one illustrative embodiment, the polypeptide(s) are detected/quantified in an electrophoretic protein separation (e.g., a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another illustrative embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and can be directly labeled or alternatively may be subsequently detected using labeled antibodies (e.g., labeled sheep anti-mouse antibodies) that specifically bind to the a domain of the antibody.

In certain embodiments, the polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte(s). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind the target peptide(s) either alone or in combination. In the case where the antibody that binds the target polypeptide(s) is not labeled, a different detectable marker, for example, an enzyme-labeled antibody capable of binding to the monoclonal antibody which binds the target polypeptide, can be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or streptavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the target polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced using standard methods well know to those of skill in the art.

It will also be recognized that antibodies can be prepared by any of a number of commercial services (e.g., Berkeley antibody laboratories, Bethyl Laboratories, Anawa, Eurogenetec, etc.).

C) Assay Optimization.

The assays described herein have immediate utility for determining whether biological effects are due to nanoparticle size and/or to other properties of the nanoparticle. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration.

Routine selection and optimization of assay formats is well known to those of ordinary skill in the art.

DI Assay Scoring.

In various embodiments, the assays of this invention level are deemed to show a positive result, when the expression level (e.g., transcription, translation) of the gene(s) is upregulated or downregulated as shown in the tables herein. In certain embodiments this is determined with respect to the level measured or known for a control sample (e.g. either a level known or measured for a normal healthy cell, tissue or organism mammal of the same species and/or sex and/or age, not exposed to the nanoparticle(s)), or a “baseline/reference” level determined at a different tissue and/or a different time. In certain embodiments, the assay(s) are deemed to show a positive result when the difference between sample and “control” is statistically significant (e.g. at the 85% or greater, preferably at the 90% or greater, more preferably at the 95% or greater and most preferably at the 98% or 99% or greater confidence level).

### Examples

The following examples are offered to illustrate, but not to limit the claimed invention.

### Example 1

### Preparation of Starting Materials

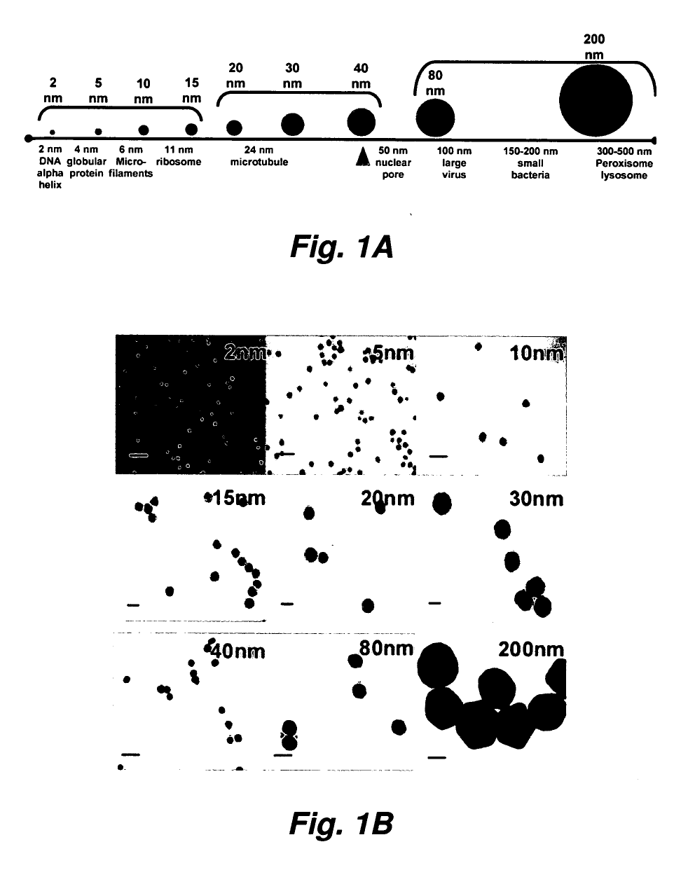

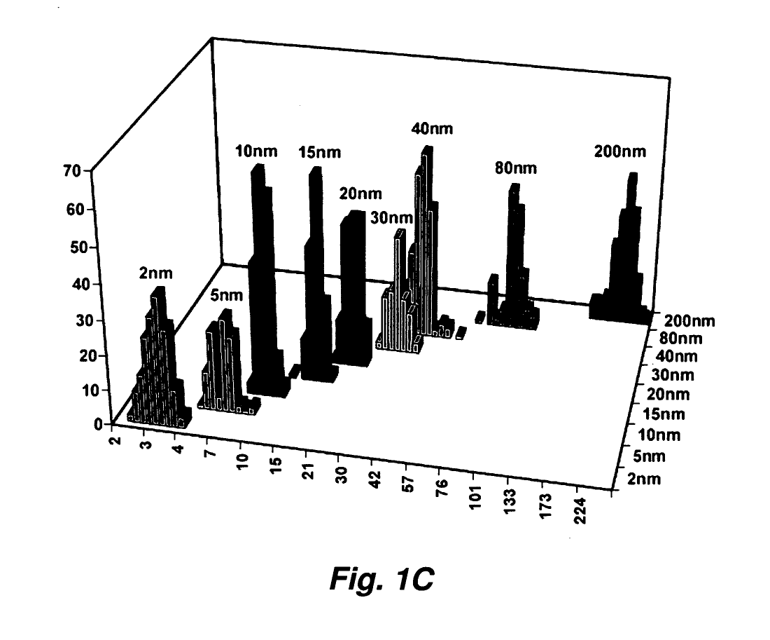

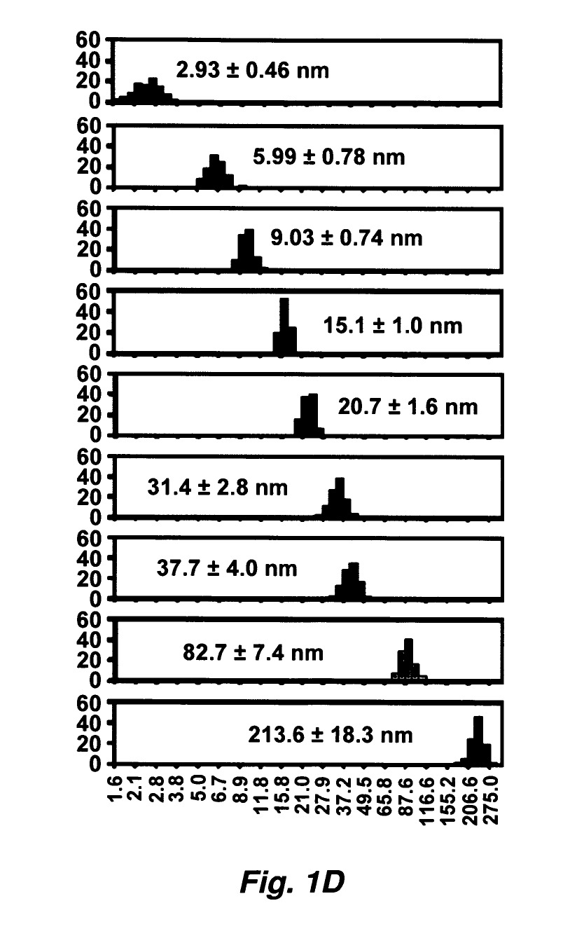

Nine different gold nanoparticle (Au-NP) sizes were used in this study that are in the same size range as molecular and cellular structures in the cell (see, e.g., FIG. 1A-1D). This size range of Au-NPs could serve as a good model system for establishing parameters that can be used to assess the properties of other nanoparticles. Although Au-NPs are more active than bulk gold (Haruta (2003) Chem Rec 3: 75-87), they are still relatively inert compared to other nanoparticles. Cellomics measurements of the treated cells indicate that there are no significant cell cycle changes induced in Au-NP treated cells, regardless of the treatment dosage (FIG. 2A, 2B). Minimal cell death (apoptosis/necrosis) is observed for a majority of the Au-NP treatments under the same condition, though 20-40 nm particles show a slight elevated apoptosis/necrosis effect consistent with previous findings on increased uptake (Chithrani and Ghazani (2006) Nano Lett 6: 662-668). Therefore, the size range of Au-NPs tested shows that they are not overtly toxic to the cells and would be an appropriate model system for studying size-dependent effects of the molecular response and signaling events.

We examined the global gene expression profiles of cells treated with Au-NPs to identify particle size-dependent molecular responses, using Illumina microarray (Barnes et al. (2005) Nucleic Acids Res 33: 5914-5923). Human Jurkat T lymphocytes were exposed to 1.2 mg/L or 0.12 mg/L of Au-NPs ranging from 2 nm to 200 nm in diameter, for either 2 or 8 hours (detail data in Supplement 1). Both size- and dose-dependent expression changes are observed. Principle Component Analysis (PCA) indicates that size and dose responses are most pronounced at the 2 hour time point (FIG. 3, panel a).

There is clear separation at 2 hours both between the two dosage groups and amongst the various Au-NP sizes within each dosage group. In the PCA graph at 2 hours (FIG. 3), the 2-40 nm size variant datasets display a near perfectly spaced gradient for both the 0.12 mg/L (FIG. 3, panel b) and 1.2 mg/L (FIG. 3, panel c) dosages. At the same time point, the 80 nm and 200 nm Au-NP treatments exhibited more separation from the smaller 2-40 nm treatments in both dosage groups (FIG. 3, panels b, c); this separation implies a major division between the 2-40 nm and the 80-200 nm gene expression patterns.

Conversely, dose and size have relatively small effects for the 8 hour treatment since no dramatic separations differentiate either the 2 dosage groups or the 9 size groups (FIG. 3, panel a). This by no means indicates the lack of size and dose effects at 8 hours given that the PCA graph here only displayed 70% of the variances in the whole gene expression dataset. Interestingly, other studies indicated that the Au-NPs are already sequestered by the cells into sub-cellular compartments at 6 hours, showing more intracellular effects (Chithrani and Ghazani (2006) Nano Lett 6: 662-668). The data here indicate that the choice of 2 hour and 8 hour time points sufficiently captured the majority of meaningful gene expression changes.

We focused on the 2 hour 0.12 mg/L dataset to illustrate the molecular mechanism in finer detail because this treatment group showed the clearest size-dependent patterns (FIG. 3, panel b). Four dominant size-dependent gene expression patterns emerge from clustering analysis (see, FIGS. 4A, 4B, 4C, and 4D) (for detailed gene lists and heatmaps, see, e.g., Tables 1-5, and FIGS. 4E-4H.

Pattern I (FIGS. 4A and 4E) contains around 11% of the differentially expressed genes that show increased down-regulation in a pseudo-linear fashion when particle size decreases, with 40-80 nm as the upper limit. These genes are functionally involved in stress response (e.g. IL18, NMI, NFATC3), DNA repair (e.g. RAD23A, XRCC2), transcription regulation, cytoskeleton organization and secretion (see, e.g., Table 1).

Pattern II (see FIGS. 4B, and 4F) represents 15% of the genes and has altered expression for only the 2 nm treatment, which also reflects the observation of overall expression change (FIG. 2C). These genes are enriched in cellular functions and processes such as transcription (e.g. FOXD1, JUND, SMAD2, SMAD3), cell growth, cell signaling, apoptosis and response to virus (see, e.g., Table 1).

Pattern III (see FIG. 4C (top and bottom panels) and FIG. 4G)v;) shows 10% of the genes responding to the 20-40 nm Au-NPs at both the 2 hour and 8 hour time-points. These genes are involved in chromosome organization and packaging, DNA packaging, DNA repair, RNA metabolism, intracellular signaling and transcription (see, e.g. Table 1). Pattern III persists over time and is the predominant expression pattern with the 0.12 mg/L Au-NP treatment at 8 hours (FIG. 2E).

Pattern IV (see, FIGS. 4D (top and bottom panels) and 4H) consists of around 7.5% of the genes that are either down-regulated or up-regulated by treatment of larger Au-NPs (80-200 nm) in both dosage groups. Interestingly, genes in this group include transcription factors such as MYC, MYCN, stress response genes, cell cycle genes and genes that are involved protein folding and transport (see, e.g., Table 1). Pattern IV is dominant at the higher 1.2 mg/L dosage of the 2 hour time point as well, indicating that there might exist a physical barrier in the cell for nanoparticles larger than 80 nm.

### Methods

TEM and Size Determination for the Au Nanoparticles

An FEI TECNAI G (Colvin (2004) Scientist 18: 26-27) transmission electron microscope (TEM) was used to determine nanoparticle size distribution (operating voltage 200 kV). The TEM samples (2, 5, 10, 15, 20, 30, 40, 80 and 200 nm) were prepared by depositing 4 uL of a diluted solution of Au-NPs onto a 3-4 nm thick film of amorphous carbon supported by a 400-mesh copper grid (Ted Pella Inc. 01822-F). Sizes of hundreds of nanoparticles from each sample were measured and analyzed using Scion Image.

Cell Culture and Cellomics

Jurkat cells were incubated at 37620 C. in humidified 5% CO₂ and treated with either different concentrations of 2 nm Au-NPs (most accessible and reactive) for 48 hours or 9 different sizes of nanoparticles at various time-points. The Cellomics measurements were performed as previously described (Ding et al. (2005) Nano Lett 5: 2448-2464).

RNA Isolation

Cells were harvested 2 or 8 hours after treatment. Triplicates of 10×10⁶ cells were used for each treatment. Cells were homogenized in TRIZOL reagent (Gibco BRL) for the isolation of total RNA, further purified with RNeasy kit (Qiagen) and then re-suspended in DEPC-treated water (SIGMA-Aldrich).

Illumina Labeling

SENTRIX® Beadchip (Illumina, Inc.) Human-6v2 arrays (48,000 transcript probes per array) were used for gene expression analysis. Each RNA sample was amplified using the Ambion Illumina RNA T7 amplification kit with biotin-UTP (Enzo) labeling. The Ambion Illumina RNA amplification kit uses a T7 oligo(dT) primer to generate single-stranded cDNA followed by second strand synthesis to generate double-stranded cDNA. In vitro transcription is done to synthesize biotin-labeled cRNA using T7 RNA polymerase. 1.5 μg of cRNA were hybridized to each array using standard Illumina protocols with streptavidin-Cy3 (Amersham) being used for detection. Slides were scanned on an Illumina Beadstation and analyzed using Beadstudio (Illumina). Data analysis has been performed using Genomatix (Genomatix Software GmbH) and Ingenuity Pathway Analysis (Ingenuity Systems) Bioinformatics 18: 207-208) software.

Clustering

The gene expression patterns, with at least once 1.5 fold changed genes from the control group across the size range of the 2 hour 0.12 mg/L treatment, were processed through the statistical package JMP (from SAS). K-Means clustering was performed according to the FOM analysis results to parse out the various expression patterns using the clustering software Genesis (Id.). The resulting clusters were further analyzed with Ingenuity Pathway Analysis, PAINT (Vadigepalli et al. (2003) Omics 7: 235-252), or Genomatix.

Promoter Analysis

The upstream promoter regions of the up- or down-regulated genes were analyzed with Genomatix. Both 500 bp upstream and 100 downstream sequences for significantly changed genes from the previous analysis were collected. The software then searched these sequences for vertebrate transcription regulatory elements to build individual interaction matrices for the individual gene lists.

Pathway Analysis

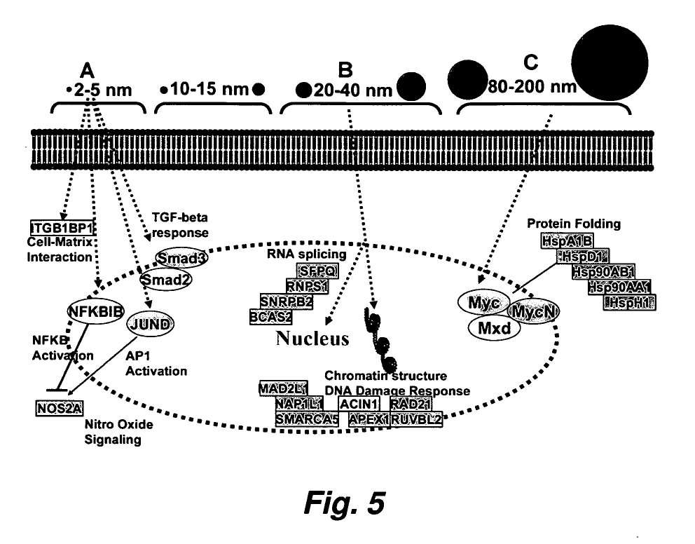

Pathway analysis was performed on the selected size-dependent expression clusters with Ingenuity Pathway Analysis. The up or down-regulated genes from the clusters were mapped to the gene objects in the Ingenuity Pathway Knowledge Base (IPKB), a mostly human-curated database of biological networks. The sub-networks (no more than 35 genes) were generated based on only “direct” interactions in the database and subsequently merged and pruned. The significance was calculated against the overall IPKB using the Fisher-exact test to determine if the function/network would be assigned by chance alone. The functional enrichments from the datasets were exported and are summarized in Table 1. The pathway results from Ingenuity and the promoter results from Genomatix were summarized in FIG. 5.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of identifying size-dependent biological effects of a nanoparticle on a cell, said method comprising:
contacting said cell with said nanoparticle;
measuring levels of gene expression in said cell of at least two genes found in one or more of Pattern Set 1, Pattern Set 2, Pattern Set 3, or Pattern Set 4;
wherein changes in expression level of said genes consistent with Pattern 1, Pattern 2, Pattern 3, or Pattern 4 is an indicator of size effects of said nanoparticle on said cell; and
wherein changes in expression level deviating from Pattern 1, Pattern 2, Pattern 3, and Pattern 4 is an indicator of biological effects that are not solely due to nanoparticle size.

2-3. (canceled)

4. The method of claim 1, wherein said measuring comprising measuring at least 50% of the genes found in Pattern Set 1, and/or Pattern Set 2, and/or Pattern Set 3, and/or Pattern Set 4.

5. (canceled)

6. The method of claim 1, wherein said measuring comprising measuring all of the genes found in Pattern Set 1, and/or Pattern Set 2, and/or Pattern Set 3, and or Pattern Set 4.

7. (canceled)

8. The method of claim 1, wherein changes in expression level of said genes consistent with Pattern 1, Pattern 2, Pattern 3, or Pattern 4 indicates that the expression level at least 75% of the measured genes is upregulated or downregulated as shown in Table 2 for pattern 1, Table 3 for pattern 2, Table 4 for pattern 3, or Table 5 for pattern 4, for particles of the same average size.

9. (canceled)

10. The method of claim 8, wherein the magnitude of upregulation or downregulation of the measured pattern set genes is comparable to the average magnitude shown in Pattern 1, Pattern 2, Pattern 3, or Pattern 4 for particles of the same size.

11. (canceled)

12. The method of claim 1, wherein said nanoparticle is a nanoparticle selected from the group consisting of a metal nanoparticle, a semiconductor nanoparticle, a polymeric nanoparticle, a dendromeric nanoparticle, a ceramic nanoparticle, a mineral nanoparticle, and a lipidic nanoparticle.

13. The method of claim 1, wherein said nanoparticle is a nanoparticle formulated for drug delivery.

14. The method of claim 12, wherein said nanoparticle further comprises a pharmaceutical.

15. The method of claim 1, wherein said contacting comprises contacting a cell in situ in a tissue or tissue section or a cell in culture.

16.-17. (canceled)

18. The method of claim 1, wherein said contacting comprises administering said nanoparticle to a non-human mammal.

19. The method of claim 1, wherein said measuring comprises measuring gene expression using a method selected from the group consisting of an array hybridization, a polymerase chain reaction (PCR), and an RT-PCR.

20-21. (canceled)

22. A method of identifying biological effects of a nanoparticle on a cell wherein said effects are not solely due to the size of said nanoparticle, said method comprising:
contacting said cell with said nanoparticle;
measuring levels of gene expression in said cell wherein changes in expression level of genes other than genes found in one or more of Pattern Set 1, Pattern Set 2, Pattern Set 3, or Pattern Set 4, or changes of expression level of genes in one or more of Pattern Set 1, Pattern Set 2, Pattern Set 3, or Pattern Set 4 deviating from Pattern 1, Pattern 2, Pattern 3, and Pattern 4 is an indicator of biological effects that are not solely due to nanoparticle size.

23. The method of claim 22, wherein said measuring comprises measuring at least three genes found in Pattern Set 1, and/or Pattern Set 2, and/or Pattern Set 3, and/or Pattern Set 4.

24.-28. (canceled)

29. The method of claim 22, wherein said measuring comprises measuring expression levels of at least two genes not found in pattern set 1, pattern set 2, pattern set 3, or pattern set 4.

30. (canceled)

31. The method of claim 22, wherein changes in expression level of said genes consistent with Pattern 1, Pattern 2, Pattern 3, or Pattern 4 indicates that the expression level at least 75% of the measured genes is upregulated or downregulated as shown in Table 2 for pattern 1, Table 3 for pattern 2, Table 4 for pattern 3, or Table 5 for pattern 4, for particles of the same average size.

32. (canceled)

33. The method of claim 31, wherein the magnitude of upregulation or downregulation of the measured pattern set genes is comparable to the average magnitude shown in Pattern 1, Pattern 2, Pattern 3, or Pattern 4 for particles of the same size.

34. (canceled)

35. The method of claim 22, wherein changes said nanoparticle is a nanoparticle selected from the group consisting of a metal nanoparticle, a semiconductor nanoparticle, a polymeric nanoparticle, a dendromeric nanoparticle, a ceramic nanoparticle, a mineral nanoparticle, and a lipidic nanoparticle.

36.-39. (canceled)

40. The method of claim 22, wherein said contacting comprises contacting comprises contacting a human cell.

41. The method of claim 22, wherein said contacting comprises administering said nanoparticle to a non-human mammal.

42. The method of claim 22, wherein said measuring comprises measuring gene expression using a method selected from the group consisting of an array hybridization, a polymerase chain reaction (PCR), and an RT-PCR.

43-44. (canceled)

45. A method of identifying genes whose expression is altered by nanoparticle size, said method comprising:
contacting a cell with a nanoparticles having different sizes; and
identifying genes whose expression level differs when exposed to at least two different size nanoparticles.

46.-52. (canceled)

53. The method of claim 45, wherein said method further comprises recording the identified genes on paper and/or on a computer readable medium.

54. A method for assessing the cytotoxic effect of a nanomaterial upon a cell, said method comprising:
exposing said cell to a nanomaterial;
detecting from said cell, the pattern of gene amplification or gene expression for at least one gene set forth in Tables 1, 2, 3, 4, 5, and/or at least one gene set forth in FIGS. 4E, 4F, 4G, or 4H, and/or in pattern set 1, pattern set 2, pattern set 3, pattern set 4, or pattern set 5 in response to said exposure;
identifying at least two-fold change in gene expression of said gene; whereby, when the two-fold change in gene expression is identified, this is an indication that the nanoparticle is cytotoxic to said cell.

55. (canceled)

56. A method for measuring size dependent biological effect of nanoparticles on a cell, said method comprising:
exposing a cell to a nanoparticle, performing gene expression profiles and gene function, promoter and pathway analyses on the cell after exposure to said nanoparticle and identifying and comparing the patterns that emerge as compared to size-dependent patterns I, II, III and IV shown in FIGS. 4A, 4B, 4C, and/or 4D, where a change in expression profile consistent with said patterns is an indicator of size dependent biological effect of said nanoparticle on said cell.

57-59. (canceled)
